(19) United States
(12) Patent Application Publication
(10) Pub. No.: US 2004/0186081 A1
(43) Pub. Date: Sep. 23, 2004

(54) Naaladase inhibitors for treating opioid tolerance

(75) Inventors: Barbara S. Slusher, Kingsville, MD (US); Krystyna Wozniak, Bel Air, MD (US)

Correspondence Address: GUILFORD PHARMACEUTICALS C/O, FOLEY & LARDNER, 3000 K STREET, NW, WASHINGTON, DC, 20007-5143, US

(73) Assignee: Guilford Pharmaceuticals Inc.

(21) Appl. No.: 10/791,296

(22) Filed: Mar. 3, 2004

Related U.S. Application Data
(60) Provisional application No. 60/450,690, filed on Mar. 3, 2003.

(51) Int. Cl. A61K 31/66

(52) U.S. Cl. 514/114; 514/120

(57) ABSTRACT

The present invention relates to pharmaceutical compositions and methods for treating opioid tolerance using NAALADase inhibitors.

This application claims the benefit of U.S. Provisional Patent Application No. 60/450,690, filed Mar. 3, 2003, the entire contents of which are herein incorporated by reference.

This invention relates to pharmaceutical compositions and methods for treating opioid tolerance using NAALADase inhibitors.

### SUMMARY OF THE INVENTION

The present invention relates to a method for treating opioid tolerance comprising administering an effective amount of a NAALADase inhibitor to a mammal in need of such treatment.

The present invention also relates to a pharmaceutical composition comprising:

(i) an effective amount of a NAALADase inhibitor for treating opioid tolerance; and

(ii) a pharmaceutically acceptable carrier.

### BRIEF DESCRIPTION OF THE DRAWINGS

FIG. 1 is a graph plotting the time courses of tail-flick responses of mice treated with a placebo, a NAALADase inhibitor, morphine, or a NAALADase inhibitor with morphine.

FIG. 2 is a bar graph plotting the mean ±S.E.M. Area Under Curve (AUC) values.

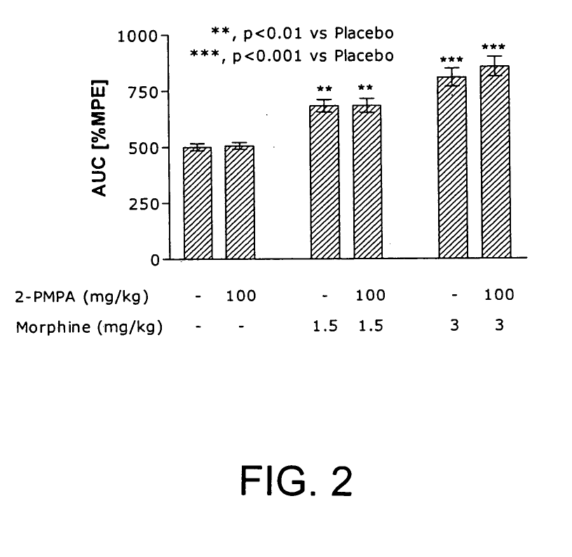

### DETAILED DESCRIPTION OF THE INVENTION

Over the last decade, research has provided compelling evidence that glutamate receptors are crucially involved in phenomena related to opioid tolerance [see (Bisaga and Popik 2000) for a recent review]. Glutamate, a major excitatory neurotransmitter in the brain, stimulates both ionotropic and metabotropic glutamate receptors (Monaghan et al. 1989; Conn and Pin 1997). Antagonists of the ionotropic N-methyl-D-aspartate (NMDA) receptor complex including memantine, the low affinity and highly voltage-dependent clinically available NMDA receptor antagonist, inhibit the development of opioid tolerance (Trujillo and Akil 1991; Marek et al. 1991; Popik et al. 2000a) and reverse preexisting tolerance so that opiate-tolerant animals treated with NMDA receptor antagonists become sensitive to doses of morphine that previously did not evoke antinociception (Tiseo and Inturrisi 1993; Popik et al. 2000a).

Another more “physiological” way of attenuating glutamate neurotransmission could potentially be achieved by inhibiting the metabolism of N-acetyl-aspartyl-glutamate (NAAG) (Slusher et al. 1999), an endogenous dipeptide present in the brain in millimolar (0.5-2.7 mM) concentrations (Pouwels and Frahm 1997) that has been immunohistochemically localized to neurons, particularly those known to be glutamatergic (Williamson and Neale 1988; Tsai et al. 1990; Tsai et al. 1993). NAAG has been hypothesized to be involved in neuronal communication as a neurotransmitter, neuromodulator and precursor of glutamate (Blakely and Coyle 1988). NAAG is released from neurons after depolarization by a calcium-dependent process upon synaptic stimulation (Tsai et al. 1990; Neale et al. 2000), suggesting its neurotransmitter-like properties.

Importantly, NAAG is hydrolyzed by the neuropeptide glutamate carboxypeptidase (GCP II; EC 3.4.17.21) [N-acetylated-alpha-linked-acidic dipeptidase (NAALADase)] to liberate N-acetyl-aspartate (NAA) and glutamate (Stauch et al. 1989), and the activity of this enzyme can be inhibited by recently developed specific inhibitors (Jackson and Slusher 2001). NAAG itself has been shown to act as a low potency agonist at NMDA receptors (Koenig et al. 1994; Sekiguchi et al. 1992; Westbrook et al. 1986), and thus, according to these data, an inhibition of its metabolism might result in stimulation of NMDA receptors. However, in many other systems it has been shown to antagonize the effects of NMDA receptor activation (Burlina et al. 1994; Grunze et al. 1996; Puttfarcken et al. 1993), and therefore, according to these findings, an inhibition of its metabolism would actually inhibit NMDA receptors. Thus, as noted by Yamamoto et al., (2001 a), NAAG acts as an NMDA receptor antagonist at low concentrations but as a low potency NMDA receptor agonist at high concentrations and can therefore be regarded as a mixed agonist/antagonist at the NMDA receptor depending on its concentration (Bruno et al. 1998; Thomas et al. 2000).

In addition, an increase in NAAG concentration may decrease glutamatergic tone mediated by presynaptic mGluRII receptors (mGluR3), because another line of evidence indicates that NAAG is a direct agonist at mGluRII receptors (Wroblewska et al. 1993; Wroblewska et al. 1997) with EC₅₀˜26 uM (Tortella et al. 2000). Other current work demonstrates effects of compounds that inhibit the function of metabotropic receptors on morphine tolerance and reward. Thus, an agonist of group II metabotropic receptors for glutamate (mGluRII), (+)-2-aminobicyclo [3,1,0] hexane-2,6-dicarboxylic acid (LY354740), has been shown to inhibit the development of morphine tolerance (Popik et al. 2000b). Increasing endogenous NAAG concentrations, via inhibition of its hydrolysis via GCPII (NAALADase) might be expected to have similar effects as exogenous addition of an mGluR3 agonist. Lastly, the inhibition of metabolism of NAAG to glutamate via use of a GCPII inhibitor, could directly produce a reduction of extracellular concentration of glutamate, and via this action, may then attenuate the stimulation of both ionotropic and metabotropic receptors for glutamate.

The pharmacological effects of inhibition of GCP II activity have not been investigated until recently, when specific and potent inhibitors of this enzyme were developed. Among them is 2-phosphonomethyl pentanedioic acid (2-PMPA) (Jackson et al. 1996) that potently inhibits GCP II activity with an inhibition constant (Kᵢ) of 0.3 nM. 2-PMPA is selective for GCP II with no apparent affinity for over 100 different receptors, ion channels, transporters, and enzymes including several glutamatergic sites such as NMDA, AMPA, metabotropic glutamate receptors and glutamate transporters (Slusher et al. 1999).

Because of its potency and apparent specificity for GCP II, we used 2-PMPA as a prototype compound to explore the role of GCP II inhibition in the development of opiod tolerance.

Definitions

“Alkyl” refers to a branched or unbranched saturated hydrocarbon chain comprising a designated number of carbon atoms. For example, C₁-C₉ alkyl is a straight or branched hydrocarbon chain containing 1 to 9 carbon atoms, and includes but is not limited to substituents such as methyl, ethyl, propyl, iso-propyl, butyl, iso-butyl, tert-butyl, n-pentyl, n-hexyl, and the like, unless otherwise indicated.

“Alkenyl” refers to a branched or unbranched unsaturated hydrocarbon chain comprising a designated number of carbon atoms. For example, C₂-C₉ alkenyl is a straight or branched hydrocarbon chain containing 2 to 9 carbon atoms having at least one double bond, and includes but is not limited to substituents such as ethenyl, propenyl, iso-propenyl, butenyl, iso-butenyl, tert-butenyl, n-pentenyl, n-hexenyl, and the like, unless otherwise indicated.

“Alkoxy” refers to the group —OR wherein R is alkyl as herein defined. In one embodiment, R is a branched or unbranched saturated hydrocarbon chain containing 1 to 9 carbon atoms.

“Carbocycle” refers to a hydrocarbon, cyclic moiety having one or more closed ring(s) that is/are alicyclic, aromatic, fused and/or bridged. Examples include cyclopropane, cyclobutane, cyclopentane, cyclohexane, cycloheptane, cyclopentene, cyclohexene, cycloheptene, cycloctene, benzyl, naphthene, anthracene, phenanthracene, biphenyl and pyrene.

“Aryl” refers to an aromatic, hydrocarbon cyclic moiety having one or more closed ring(s). Examples include, without limitation, phenyl, naphthyl, anthracenyl, phenanthracenyl, biphenyl and pyrenyl.

“Heterocycle” refers to a cyclic moiety having one or more closed ring(s) that is/are alicyclic, aromatic, fused and/or bridged, with one or more heteroatom(s) (for example, sulfur, nitrogen or oxygen) in at least one of the rings. Examples include, without limitation, pyrrolidine, pyrrole, thiazole, thiophene, piperidine, pyridine, isoxazolidine and isoxazole.

“Heteroaryl” refers to an aromatic, cyclic moiety having one or more closed ring(s) with one or more heteroatom(s) (for example, sulfur, nitrogen or oxygen) in at least one of the rings. Examples include, without limitation, pyrrole, thiophene, pyridine and isoxazole.

“Linking group” refers to a moiety that connects the terminal group with the benzene ring in the compounds of formula VI, without compromising with the pharmacological or biological activity of the overall compound.

“Metal binding group” refers to a functional group capable of interacting with metal ion(s), such as Co²⁺, Ni²⁺, Mn²⁺, Cu²⁺, Zn²⁺, Mg²⁺, Fe²⁺, Fe³⁺, or Al³⁺.Common metal binding groups include amines (e.g. ethylenediamine), aldehydes, ketones, carboxylic acids (e.g. ethylenediaminetetraacetic acid (EDTA)), thiols, phosphorus derivatives and hydroxamic acids.

“Derivative” refers to a substance produced from another substance either directly or by modification or partial substitution.

“Effective amount” refers to the amount required to produce the desired effect.

“Therapeutically effective amount” refers to the amount required to treat glaucoma in an animal or a mammal.

“Halo” refers to at least one fluoro, chloro, bromo or iodo moiety.

“Isosteres” refer to elements, functional groups, substitutents, molecules or ions having different molecular formulae but exhibiting similar or identical physical properties. For example, tetrazole is an isostere of carboxylic acid because it mimics the properties of carboxylic acid even though they both have different molecular formulae. Typically, two isosteric molecules have similar or identical volumes and shapes. Ideally, isosteric compounds should be isomorphic and able to co-crystallize. Other physical properties that isosteric compounds usually share include boiling point, density, viscosity and thermal conductivity. However, certain properties are usually different: dipolar moments, polarity, polarization, size and shape since the external orbitals may be hybridized differently. The term “isosteres” encompass “bioisosteres”.

“Bioisosteres” are isosteres that, in addition to their physical similarities, share some common biological properties. Typically, bioisosteres interact with the same recognition site or produce broadly similar biological effects.

“Carboxylic acid isosteres” include without limitation direct derivatives such as hydroxamic acids, acyl-cyanamides and acylsulfonamides; planar acidic heterocycles such as tetrazoles, mercaptoazoles, sulfinylazoles, sulfonylazoles, isoxazoles, isothiazoles, hydroxythiadiazoles and hydroxychromes; and nonplanar sulfur- or phosphorus-derived acidic functions such as phosphinates, phosphonates, phosphonamides, sulphonates, sulphonamides, and acylsulphonamides.

“Metabolite” refers to an intermediate or product resulting from metabolism.

“NAAG” refers to N-acetyl-aspartyl-glutamate, an important peptide component of the brain, with levels comparable to the major inhibitor neurotransmitter gamma-aminobutyric acid (GABA). NAAG is neuron-specific, present in synaptic vesicles and released upon neuronal stimulation in several systems presumed to be glutamatergic. Studies suggest that NAAG may function as a neurotransmitter and/or neuromodulator in the central nervous system, or as a precursor of the neurotransmitter glutamate. In addition, NAAG is an agonist at group II metabotropic glutamate receptors, specifically mGluR3 receptors; when attached to a moiety capable of inhibiting NAALADase, it is expected that metabotropic glutamate receptor ligands will provide potent and specific NAALADase inhibitors.

“NAALADase” refers to N-acetylated α-linked acidic dipeptidase, a membrane bound metallopeptidase that catabolizes NAAG to N-acetylaspartate (“NAA”) and glutamate (“GLU”):

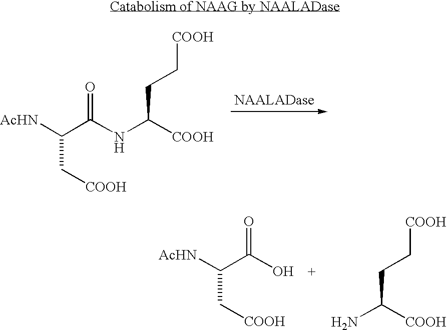

NAALADase has been assigned to the M28 peptidase family and is also called prostate specific membrane antigen (PSM) or human glutamate carboxypeptidase II (GCP II), EC number 3.4.17.21. It is believed that NAALADase is a co-catalytic zinc/zinc metallopeptidase. NAALADase shows a high affinity for NAAG with a Km of 540 nM. If NAAG is a bioactive peptide, then NAALADase may serve to inactivate NAAG'S synaptic action. Alternatively, if NAAG functions as a precursor for glutamate, the primary function of NAALADase may be to regulate synaptic glutamate availability.

“Pharmaceutically acceptable carrier” refers to any carrier, diluent, excipient, wetting agent, buffering agent, suspending agent, lubricating agent, adjuvant, vehicle, delivery system, emulsifier, disintegrant, absorbent, preservative, surfactant, colorant, flavorant, or sweetener that would be suitable for use in a pharmaceutical composition. In one embodiment, the pharmaceutically acceptable carrier is non-toxic.

“Pharmaceutically acceptable equivalent” includes, without limitation, pharmaceutically acceptable salts, hydrates, metabolites, prodrugs, and isosteres. Many pharmaceutically acceptable equivalents are expected to have the same or similar in vitro or in vivo activity as the inventive compounds.

“Pharmaceutically acceptable salt” refers to a salt of the inventive compounds that possesses the desired pharmacological activity and that is neither biologically nor otherwise undesirable. The salt can be formed with acids that include without limitation acetate, adipate, alginate, aspartate, benzoate, benzenesulfonate, bisulfate butyrate, citrate, camphorate, camphorsulfonate, cyclopentanepropionate, digluconate, dodecylsulfate, ethanesulfonate, flumarate, glucoheptanoate, glycero-phosphate, hemisulfate, heptanoate, hexanoate, hydrochloride hydrobromide, hydroiodide, 2-hydroxyethane-sulfonate, lactate, maleate, methanesulfonate, 2-naphthalenesulfonate, nicotinate, oxalate, thiocyanate, tosylate and undecanoate. Examples of a base salt include ammonium salts, alkali metal salts such as sodium and potassium salts, alkaline earth metal salts such as calcium and magnesium salts, salts with organic bases such as dicyclohexylamine salts, N-methyl-D-glucamine, and salts with amino acids such as arginine and lysine. The basic nitrogen-containing groups can be quarternized with agents including lower alkyl halides such as methyl, ethyl, propyl and butyl chlorides, bromides and iodides; dialkyl sulfates such as dimethyl, diethyl, dibutyl and diamyl sulfates; long chain halides such as decyl, lauryl, myristyl and stearyl chlorides, bromides and iodides; and aralkyl halides such as benzyl and phenethyl bromides.

“Prodrug” refers to a derivative of the inventive compounds that undergoes biotransformation, such as metabolism, before exhibiting its pharmacological effect(s). The prodrug is formulated with the objective(s) of improved chemical stability, improved patient acceptance and compliance, improved bioavailability, prolonged duration of action, improved organ selectivity, improved formulation (e.g., increased hydrosolubility), and/or decreased side effects (e.g., toxicity). The prodrug can be readily prepared from the inventive compounds using methods known in the art, such as those described by Burger's Medicinal Chemistry and Drug Chemistry, Fifth Ed., Vol. 1, pp. 172-178, 949-982 (1995).

“Inhibition,” in the context of enzymes, refers to reversible enzyme inhibition such as competitive, uncompetitive and non-competitive inhibition. Competitive, uncompetitive and non-competitive inhibition can be distinguished by the effects of an inhibitor on the reaction kinetics of an enzyme. Competitive inhibition occurs when the inhibitor combines reversibly with the enzyme in such a way that it competes with a normal substrate for binding at the active site. The affinity between the inhibitor and the enzyme may be measured by the inhibitor constant, Kᵢ, which is defined as:

$${K}_{i} = \frac{[ E ] [ I ]}{[ EI ]}$$

wherein [E] is the concentration of the enzyme, [I] is the concentration of the inhibitor, and [EI] is the concentration of the enzyme-inhibitor complex formed by the reaction of the enzyme with the inhibitor. Unless otherwise specified, Kᵢ as used herein refers to the affinity between the inventive compounds and NAALADase. “IC₅₀” is a related term used to define the concentration or amount of a compound that is required to cause a 50% inhibition of the target enzyme.

“NAALADase inhibitor” refers to any compound that inhibits NAALADase enzyme activity. Embodiments include a NAALADase inhibitor that exhibits a Kᵢ of less than 100 μM, less than 10 μM, and less than 1 μM, as determined using any appropriate assay known in the art.

“Isomers” refer to compounds having the same number and kind of atoms, and hence the same molecular weight, but differing in respect to the arrangement or configuration of the atoms.

“Optical isomers” refer to enantiomers or diastereoisomers.

“Stereoisomers” are isomers that differ only in the arrangement of the atoms in space.

“Diastereoisomers” are stereoisomers that are not mirror images of each other. Diastereoisomers occur in compounds having two or more asymmetric carbon atoms; thus, such compounds have 2ⁿ optical isomers, where n is the number of asymmetric carbon atoms

“Enantiomers” are a pair of stereoisomers that are non-superimposable mirror images of each other. Enantiomers result, for example, from the presence of one or more asymmetric carbon atom(s) in the compound (e.g., glyceraldehyde, lactic acid, sugars, tartaric acid, amino acids).

“Enantiomer-enriched” refers to a mixture in which one enantiomer predominates.

“Racemic mixture” means a mixture containing equal amounts of enantiomers.

“Non-racemic mixture” is a mixture containing unequal amounts of enantiomers.

“Animal” refers to a living organism having sensation and the power of voluntary movement, and which requires for its existence oxygen and organic food. Examples include, without limitation, members of the human, equine, porcine, bovine, murine, canine, or feline species. In the case of a human, an “animal” may also be referred to as a “patient”.

“Mammal” refers to a warm-blooded vertebrate animal.

“Opioid” refers to a narcotic analgesic that is either semi or fully synthetic, including, but not limited to Codeine, Morphine, Heroin, Hydromorphone (Dilaudid), Oxycodone (Percodan), Oxymorphone (Numorphan), Hydrocodone (Vicodin), Meperidine (Demerol), Fentanyl, Methadone (Dolophine), Darvon, Talwin.

“Opioid tolerance” includes without limitation the failure of a steady dose of the drug over time, to sustain the desired pharmacological effect, i.e., the need to increase the drug dosage to maintain the original pharmacological effect.

“Treating” refers to:

(i) preventing a disease, disorder or condition from occurring in an animal that may be predisposed to the disease, disorder and/or condition but has not yet been diagnosed as having it;

(ii) inhibiting the disease, disorder or condition, i.e., arresting its development; and/or

(iii) relieving the disease, disorder or condition, i.e., causing regression of the disease, disorder and/or condition.

“Treating opioid tolerance” refers to:

(i) preventing opioid tolerance from occurring in an animal that may be predisposed to opioid tolerance but has not yet been diagnosed as having it;

(ii) inhibiting or slowing opioid tolerance, e.g. arresting its development; and/or

(iii) relieving opioid tolerance, e.g. causing its regression.

One of ordinary skill in the art would recognize that there are alternative nomenclatures, nosologies and classification systems for the diseases, disorders and conditions defined above, and that such systems evolve with medical scientific progress.

Unless the context clearly dictates otherwise, the definitions of singular terms may be extrapolated to apply to their plural counterparts as they appear in the application; likewise, the definitions of plural terms may be extrapolated to apply to their singular counterparts as they appear in the application.

Methods of the Present Invention

The present invention relates to a method for treating opioid tolerance comprising administering an effective amount of a NAALADase inhibitor to an animal or a mammal in need of such treatment.

Pharmaceutical Compositions of the Present Invention

The present invention further relates to a pharmaceutical composition comprising:

(i) an effective amount of a NAALADase inhibitor for treating opioid tolerance; and

(ii) a pharmaceutically acceptable carrier.

Formula I

An example of a NAALADase inhibitor is a compound of formula I:

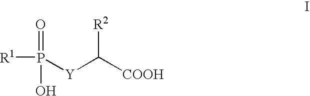

or an enantiomer or a pharmaceutically acceptable equivalent of said compound, wherein:

Y is CR³R⁴, NR⁵ or O;

R¹ is hydrogen, C₁-C₉ alkyl, C₂-C₉ alkenyl, C₃-C₈ cycloalkyl, C₅-C₇ cycloalkenyl, Ar, COOR⁶, NR⁶R⁷ or OR⁶, wherein said alkyl, alkenyl, cycloalkyl and cycloalkenyl are independently unsubstituted or substituted with one or more substituent(s) which are, for example, independently selected from carboxy, C₃-C₈ cycloalkyl, C₅-C₇ cycloalkenyl, halo, hydroxy, nitro, trifluoromethyl, C₁-C₆ alkyl, C₂-C₆ alkenyl, C₁-C₉ alkoxy, C₂-C₉ alkenyloxy, phenoxy, benzyloxy, COOR⁶, NR⁶R⁷ and Ar;

R² is hydrogen, C₁-C₆ alkyl, C₂-C₆ alkenyl, C₃-C₈ cycloalkyl, C₅-C₇ cycloalkenyl, Ar, halo or carboxy, wherein said alkyl, alkenyl, cycloalkyl and cycloalkenyl are independently unsubstituted or substituted with one or more substituent(s) which are, for example, independently selected from carboxy, C₃-C₈ cycloalkyl, C₅-C₇ cycloalkenyl, halo, hydroxy, nitro, trifluoromethyl, C₁-C₆ alkyl, C₂-C₆ alkenyl, C₁-C₉ alkoxy, C₂-C₉ alkenyloxy, phenoxy, benzyloxy, NR⁶R⁷ and Ar;

R³ and R⁴ are independently hydrogen or C₁-C₃ alkyl;

R⁵is hydrogen or C₁-C₃ alkyl;

R⁶ and R⁷ are independently hydrogen, C₁-C₉ alkyl, C₂-C₉ alkenyl, C₃-C₈ cycloalkyl, C₅-C₇ cycloalkenyl or Ar, wherein said alkyl, alkenyl, cycloalkyl and cycloalkenyl are independently unsubstituted or substituted with one or more substituent(s) which are, for example, independently selected from carboxy, C₃-C₈ cycloalkyl, C₅-C₇ cycloalkenyl, halo, hydroxy, nitro, trifluoromethyl, C₁-C₆ alkyl, C₂-C₆ alkenyl, C₁-C₉ alkoxy, C₂-C₉ alkenyloxy, phenoxy, benzyloxy and Ar; and

Ar is selected from 1-naphthyl, 2-naphthyl, 2-indolyl, 3-indolyl, 4-indolyl, 2-furyl, 3-furyl, tetrahydrofuranyl, tetrahydropyranyl, 2-thienyl, 3-thienyl, 2-pyridyl, 3-pyridyl, 4-pyridyl and phenyl, wherein said Ar is unsubstituted or substituted with one or more substituent(s) which are, for example, independently selected from halo, hydroxy, nitro, trifluoromethyl, C₁-C₆ alkyl, C₂-C₆ alkenyl, C₁-C₆ alkoxy, C₂-C₆ alkenyloxy, phenoxy, benzyloxy, carboxy and N⁶R⁷.

In one embodiment of formula I, Y is CH₂.

In another embodiment, R² is —(CH₂)₂COOH.

In a further embodiment, R¹ is hydrogen, C₁-C₄ alkyl, C₂-C₄ alkenyl, C₃-C₈ cycloalkyl, C₅-C₇ cycloalkenyl, benzyl, phenyl or OR⁶, wherein said alkyl, alkenyl, cycloalkyl, cycloalkenyl, benzyl and phenyl are independently unsubstituted or substituted with one or more substituent(s) independently selected from carboxy, C₃-C₈ cycloalkyl, C₅-C₇ cycloalkenyl, halo, hydroxy, nitro, trifluoromethyl, C₁-C₆ alkyl, C₂-C₆ alkenyl, C₁-C₆ alkoxy, C₂-C₆ alkenyloxy, phenoxy, benzyloxy, NR⁶R⁷, benzyl and phenyl.

Examples of compounds of formula I include without limitation:

2-(phosphonomethyl)pentanedioic acid;

2-[[(2-carboxyethyl)hydroxyphosphinyl]methyl]-pentanedioic acid;

2-[(benzylhydroxyphosphinyl)methyl]pentanedioic acid;

2-[(phenylhydroxyphosphinyl)methyl]pentanedioic acid;

2-[[((hydroxy)phenylmethyl)hydroxyphosphinyl]-methyl]pentanedioic acid;

2-[(butylhydroxyphosphinyl)methyl]pentanedioic acid;

2-[[(3-methylbenzyl)hydroxyphosphinyl]methyl]-pentanedioic acid;

2-[(3-phenylpropylhydroxyphosphinyl)methyl]-pentanedioic acid;

2-[[(4-fluorophenyl)hydroxyphosphinyl]methyl]-pentanedioic acid;

2-[(methylhydroxyphosphinyl)methyl]pentanedioic acid;

2-[(phenylethylhydroxyphosphinyl)methyl]pentanedioic acid;

2-[[(4-methylbenzyl)hydroxyphosphinyl]methyl]-pentanedioic acid;

2-[[(4-fluorobenzyl)hydroxyphosphinyl]methyl]-pentanedioic acid;

2-[[(4-methoxybenzyl)hydroxyphosphinyl]methyl]-pentanedioic acid;

2-[[(3-trifluoromethylbenzyl)hydroxyphosphinyl]-methyl]pentanedioic acid;

2-[[4-trifluoromethylbenzyl)hydroxyphosphinyl]-methyl]pentanedioic acid;

2-[[(2-fluorobenzyl)hydroxyphosphinyl]methyl]-pentanedioic acid;

2-[[(2,3,4,5,6-pentafluorobenzyl)hydroxy-phosphinyl]methyl]pentanedioic acid; and

enantiomers and pharmaceutically acceptable equivalents.

Formula II

Another NAALADase inhibitor is a compound of formula II

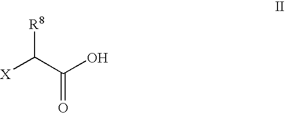

or an enantiomer or a pharmaceutically acceptable equivalent of said compound, wherein:

X is a moiety of formula III, IV or V

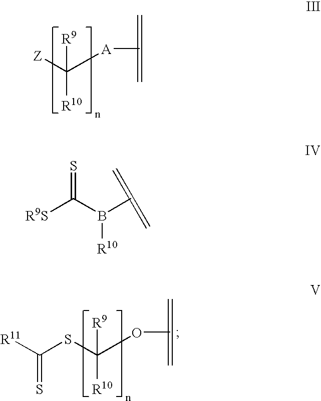

Z is SH, SO_{3H}, SO₂H, SOH, SO(NH)R¹² or S(NHR¹²)₂R¹³;

B is N or CR¹⁴;

A is O, S, CR¹⁵R¹⁶ or (CR¹⁵R¹⁶)ₘS;

m and n are independently 0, 1, 2, 3 or 4;

R⁸, R⁹, R¹⁰, R¹¹, R¹², R¹⁴, R¹⁵ and R¹⁶ are independently hydrogen, C₁-C₉ alkyl, C₂-C₉ alkenyl, C₃-C₈ cycloalkyl, C₅-C₇ cycloalkenyl, Ar¹, hydroxy, carboxy, carbonyl, amino, cyano, isocyano, nitro, sulfonyl, sulfoxy, thio, thiocarbonyl, thiocyano, formanilido, thioformamido, sulfhydryl, halo, haloalkyl, trifluoromethyl or oxy, wherein said alkyl, alkenyl, cycloalkyl and cycloalkenyl are independently unsubstituted or substituted with one or more substituent(s); and

Ar¹ is a carbocyclic or heterocyclic moiety, which is unsubstituted or substituted with one or more substituent(s);

provided that when X is a moiety of formula III and A is O, then n is 2, 3 or 4; when X is a moiety of formula III and A is S, then n is 2, 3 or 4; and when X is a moiety of formula III and A is (CR¹⁵R¹⁶)ₘS, then n is 0, 2, 3 or 4.

In one embodiment of formula II, X is a moiety of formula II; n is 0, 1, 2 or 3; Z is SH, SO₃H, SO₂H, SOH or S(NHR¹²)₂R¹³; and A is O, S or CR¹⁵R¹⁶.

In another embodiment, R is —(CH₂)₂COOH.

In a further embodiment, Z is SH.

Examples of compounds of formula II include without limitation:

2-(2-sulfanylethyl)pentanedioic acid;

3-(2-sulfanylethyl)-1,3,5-pentanetricarboxylic acid;

2-(2-sulfanylpropyl)pentanedioic acid;

2-(2-sulfanylbutyl)pentanedioic acid;

2-(2-sulfanyl-2-phenylethyl)pentanedioic acid;

2-(2-sulfanylhexyl)pentanedioic acid;

2-(2-sulfanyl-1-methylethyl)pentanedioic acid;

2-[1-(sulfanylmethyl)propyl]pentanedioic acid;

2-(3-sulfanylpentyl)pentanedioic acid;

2-(3-sulfanylpropyl)pentanedioic acid;

2-(3-sulfanyl-2-methylpropyl)pentanedioic acid;

2-(3-sulfanyl-2-phenylpropyl)pentanedioic acid;

2-(3-sulfanylbutyl)pentanedioic acid;

2-[3-sulfanyl-2-(phenylmethyl)propyl]pentanedioic acid;

2-[2-(sulfanylmethyl)butyl]pentanedioic acid;

2-[2-(sulfanylmethyl)pentyl]pentanedioic acid;

2-(3-sulfanyl-4-methylpentyl)pentanedioic acid; and

enantiomers and pharmaceutically acceptable equivalents.

Formula VI

Another NAALADase inhibitor is a compound of formula VI

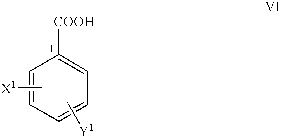

or an enantiomer or a pharmaceutically acceptable equivalent of said compound, wherein:

X¹ is —W-Z¹;

W is a bond or a linking group;

Z¹ is a terminal group; and

Y¹ is —COOH oriented meta or para relative to C-1.

Linking groups include, without limitation, divalent hydrocarbon chains, ethers, sulfides and amines, wherein the hydrocarbon chain, whether alone or part of the ether, sulfide or amine, may be saturated or unsaturated, straight or branched, open or closed, unsubstituted or substituted with one or more substituent(s) which are, for example, independently selected from C₁-C₆ alkoxy, C₂-C₆ alkenyloxy, phenoxy, benzyloxy, hydroxy, carboxy, carbamido, carbamoyl, carbamyl, carbonyl, carbozoyl, amino, hydroxyamino, formamido, formyl, guanyl, cyano, cyanoamino, isocyano, isocyanato, diazo, azido, hydrazino, triazano, nitro, nitroso, isonitroso, nitrosamino, imino, nitrilo, isonitrilo, nitrosimino, oxo, C₁-C₆ alkylthio, sulfamino, sulfamoyl, sulfeno, sulfhydryl, sulfinyl, sulfo, sulfonyl, sulfoxy, thiocarboxy, thiocyano, isothiocyano, thioformamido, halo, haloalkyl, chlorosyl, chloryl, perchloryl, trifluoromethyl, iodosyl, iodyl, phosphino, phosphinyl, phospho, phosphono, arsino, selanyl, diselanyl, siloxy, silyl and silylene.

W is a bond, —(CR¹⁷R¹⁸)ₙ-, —(CR¹⁷R¹⁸)ₙO(CR¹⁹R²⁰)ₘ-, —(CR¹⁷R¹⁸)ₙS(CR¹⁹R²⁰)ₘ- or —(CR¹⁷R¹⁸)ₙNR²¹(CR¹⁹R²⁰)ₘ-, wherein m and n are independently 0-9, and R¹⁷, R¹⁸, R¹⁹, R²⁰ and R²¹ are independently hydrogen, C₁-C₆ alkyl, C₂-C₆ alkenyl, C₂-C₆ alkynyl, C₆-C₁₄ aryl, heteroaryl, C₆-C₁₄ carbocycle, heterocycle, halo, hydroxy, sulfhydryl, nitro, amino or C₁-C₆ alkoxy, and said alkyl, alkenyl, alkynyl, aryl, heteroaryl, carbocycle, heterocycle and alkoxy are independently unsubstituted or substituted with one or more substituent(s). In one embodiment, R¹⁷, R¹⁸, R¹⁹, R²⁰ and R²¹ are each hydrogen and the total number of carbon atoms in W is 2-6.

Z¹ is a metal binding group. In one embodiment, Z¹ is —COOH, —COR²², —OR²², —CF₃, —CN, —F, —Cl, —Br, —I, —NO, —NO₂, —C(O)(NR²²OR²³), —C(O)(NR²²PO₃H₂), —C(O)(NR²²R²³), ═NOH, —NR²²(P(O)(R²³)OH), ═NR²², —N═NR²², —N(R²²)CN, —N²²(CR²³R²⁴)ₚCOOH, —NR²²(CO)NR²³R²⁴, —NR²²(COOR²³), —NR²²(CO)R²³, —NR²²(OR²³), —NR²²R²³, —NR²²(SO₂R²³), —O(CO)R²², —OR²², —SO₂(OR²²), —SO₂(N²²R²³), —SO₂R²², —SO₃R²², —SNR²²(OR²³), —S(NR²²R²³), —SR²², —SSR²², —P(O)(OH)OR²², —P(O)(OH)R²² or —PR²²R²³, wherein p is 0-6, and R²², R²³ and R²⁴ are independently hydrogen, C₁-C₉ alkyl, C₂-C₉ alkenyl, C₂-C₉ alkynyl, C₆-C,₄ aryl, heteroaryl, C₆-C₁₄ carbocycle, heterocycle, halo, hydroxy, sulfhydryl, nitro, amino or C₁-C₉ alkoxy, and said alkyl, alkenyl, alkynyl, aryl, heteroaryl, carbocycle, heterocycle and alkoxy are independently unsubstituted or substituted with one or more substituent(s). In another embodiment, Z¹ is —NH(CR²³R²⁴)ₚCOOH, —PO(OH)OR²², —PO(OH)R²², —NR²²(P(O)(R²³)OH), —CON(R²²)(OH) or —SH.

In one embodiment of formula VI:

X¹ is —(CR¹⁷R¹⁸)ₙNH(CR¹⁹R²⁰)ₘCOOH, —PO(OH)OR²², —(CR¹⁷R¹⁸)ₙP(O)(OH)R²², —NH—(CR¹⁹R²⁰)ₘ-heteroaryl, —NH(P(O)(R²³)OH), —(CR¹⁷R¹⁸)ₙNH(P(O)(OH)R²³), —CON(R²²)(OH)—(CR¹⁷CR¹⁸)ₙCON(R²²)(0H), —(CR¹⁷R¹⁸)ₙSH or —O(CR¹⁹R²⁰)ₘSH, —SO₂NH-aryl, —N(C═O)-CH₂(C═O)-aryl, —SO₂NH-aryl, —N(C═O)—CH₂(C═O)-aryl, -O-aryl wherein aryl in -O-aryl is substituted by at least one of nitro, carboxy or

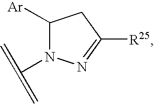

wherein X¹ is oriented meta or para relative to C-1;

m and n are independently 1-3, provided that when X¹ is —O(CR¹⁹R²⁰)ₘSH, then m is 2 or 3;

R¹⁷, R¹⁸, R¹⁹, R²⁰, R²², R²³ and R²⁵ are independently hydrogen, C₁-C₆ alkyl, C₂-C₆ alkenyl, C₂-C₆ alkynyl, aryl, heteroaryl, carbocycle, heterocycle, halo, hydroxy, sulfhydryl, nitro, amino or C₁-C₆ alkoxy, wherein said alkyl, alkenyl, alkynyl, aryl, heteroaryl, carbocycle, heterocycle and alkoxy are independently unsubstituted or substituted with one or more substituent(s); and

Y¹ is —COOH oriented meta or para relative to C-1.

When X is —PO(OH)OR²² or —(CR¹⁷R¹⁸)ₙP(O)(OH)OR²², then R²² is not H or methyl; when X is —NH(P(O)(R²³)OH or —(CR¹⁷R¹⁸)ₙNH(P(O)(OH)R²³), then R²³ is not benzyl unsubstituted or substituted with amino; and when X is —CON(R²²)(OH), then R²² is not H or methyl.

In another embodiment of formula VI, X¹ is oriented meta relative to C-1, and Y¹ is oriented ortho relative to X¹ and para relative to C-1. In a further embodiment, W is a bond, —(CH₂)ₙ-NH—(CH₂)ₘ- or —(CH₂)ₙ-; m is 1-3; n is 0-3; and Z¹ is —CO₂H, —NO₂, —NH₂, —SO₃H, halo, C₅-C₆ heteroaryl, carboxyphenylthio, or mono- or di-carboxyphenylsulfonyl.

Examples of this embodiment include:

2-[(4-carboxyphenyl)sulfonyl]-1,4-benzene-dicarboxylic acid;

2-[(2,5-dicarboxyphenyl)sulfonyl]-1,4-benzene-dicarboxylic acid;

1,2,4-benzenetricarboxylic acid;

2-[(2-carboxyphenyl)thio]-1,4-benzenedicarboxylic acid;

2-nitro-1,4-benzenedicarboxylic acid;

2-bromo-1,4-benzenedicarboxylic acid;

2-amino-1,4-benzenedicarboxylic acid;

2-sulfoterephthalic acid, monosodium salt;

2-carboxymethyl- 1,4-benzenedicarboxylic acid;

2-[(2-furanylmethyl)-amino]-1,4-benzenedicarboxylic acid;

2-[(carboxymethyl)amino]-i,4-benzenedicarboxylic acid; and

enantiomers and pharmaceutically acceptable equivalents.

In another embodiment of formula VI, X¹ is oriented ortho relative to C-1, and Y¹ is oriented para relative to X¹ and meta relative to C-1. In one embodiment, (1) when W is a bond, then Z¹ is —CO₂H, —OH, —NO₂, —C(O)(NHR²³), —SR²³, —COR²³ or —NH(CH₂R²³), and R²³ is an aryl or a heteroaryl wherein said aryl and heteroaryl are independently unsubstituted or substituted with one or more alkyl, nitro or carboxy group(s); and (2) when W is —(CH₂)ₙ- and n is 1-3, then Z¹ is —SH.

Examples of this embodiment include:

4-(4-nitrobenzoyl)-1,3-benzenedicarboxylic acid;

4-[4-(2,4-dicarboxybenzoyl)phenoxy]-1,2-benzene-dicarboxylic acid;

4-[[(2,4,6-trimethylphenyl)amino]carbonyl]-1,3-benzenedicarboxylic acid;

4-nitro-1,3-benzenedicarboxylic acid;

4-[(1-naphthalenylamino)-carbonyl]-1,3-benzene-dicarboxylic acid;

1,2,4-benzenetricarboxylic acid;

4-[(2-carboxyphenyl)thio]-1,3-benzenedicarboxylic acid;

4-[3-[[3-(2,4-dicarboxyphenoxy)propyl]dithio]-propoxy]-1,3-benzenedicarboxylic acid;

4-hydroxy-1,3-benzenedicarboxylic acid;

4-[(2-furanylmethyl)amino]-1,3-benzenedicarboxylic acid;

4-(2-mercaptoethyl)-1,3-benzenedicarboxylic acid; and

enantiomers and pharmaceutically acceptable equivalents.

In another embodiment of formula VI, X¹ is oriented meta relative to C-1, and Y¹ is oriented meta relative to X¹ and meta relative to C-1. In one embodiment, (1) when W is a bond, —(CH₂)ₙ- or —O(CH₂)ₘ- and m and n are independently 0-3, then Z¹ is —SO₃H, —NO₂, —NH₂, —CO₂H, —OH, —PO₃H, —CO(NHOH) or —SH; (2) when W is —(CH₂)ₙNH(CH₂)ₘ- and m and n are independently 0-3, then Z¹ is —CO₂H or C₅-C₆ heteroaryl; and (3) when W is a bond, then Z¹ is either (a) a heteroaryl that is unsubstituted or substituted with an aryl that is unsubstituted or substituted with one or more C₁-C₃ alkyl, halo, nitro or hydroxy group(s), or (b) —SO₂(NHR²⁴) or —NH(COR²⁴), wherein R²⁴ is an aryl that is unsubstituted or substituted with one or more nitro, amino, halo or hydroxy group(s).

Examples of this embodiment include:

5-[4,5-dihydro-5-(4-hydroxyphenyl)-3-phenyl-1 H-pyrazol-1-yl]-1,3-benzenedicarboxylic acid;

5-(4,5-dihydro-3-methyl-5-phenyl-1 H-pyrazol-1-yl)-1,3-benzenedicarboxylic acid;

5-[[(4-chloro-3-nitrophenyl)amino]sulfonyl]-I,3-benzenedicarboxylic acid;

5-[[[4-chloro-3-[[3-(2-methoxyphenyl)-1,3-dioxopropyl]amino]phenyl]amino]sulfonyl-1,3-benzenedicarboxylic acid;

5-[[3-[4-(acetylamino)phenyl]-1,3-dioxopropyl]amino]-1,3-benzenedicarboxylic acid;

5-acetylamino-1,3-benzenedicarboxylic acid;

5-[[(1-hydroxy-2-naphthalenyl)carbonyl]-methylamino]-1,3-benzenedicarboxylic acid;

5-(4-carboxy-2-nitrophenoxy)-1,3-benzenedicarboxylic acid;

5-sulfo-1,3-benzenedicarboxylic acid;

5-nitro-1,3-benzenedicarboxylic acid;

5-amino-1,3-benzenedicarboxylic acid;

1,3,5-benzenetricarboxylic acid;

5-[[(3-amino-4-chlorophenyl)amino]sulfonyl]-1,3-benzenedicarboxylic acid;

5-(3-mercaptopropoxy)-1,3-benzenedicarboxylic acid;

5-hydroxy-1,3-benzenedicarboxylic acid;

5-(2-mercaptoethoxy)-1,3-benzenedicarboxylic acid;

5-[(hydroxyamino)carbonyl]-1,3-benzenedicarboxylic acid;

5-phosphono-1,3-benzenedicarboxylic acid;

5-mercaptomethyl-1,3-benzenedicarboxylic acid;

5-phosphonomethyl-1,3-benzenedicarboxylic acid;

5-[[(carboxymethyl)amino]-methyl]-1,3-benzene-dicarboxylic acid;

5-[(carboxymethyl)amino]-1,3-benzenedicarboxylic acid;

5-[[(2-furanylmethyl)amino]-methyl]-1,3-benzene-dicarboxylic acid;

5-[2-(hydroxyamino)-2-oxoethyl]-1,3-benzene-dicarboxylic acid;

5-(2-mercaptoethyl)-1,3-benzenedicarboxylic acid; and

enantiomers and pharmaceutically acceptable equivalents.

Formula VII

Another NAALADase inhibitor is a compound of formula VII

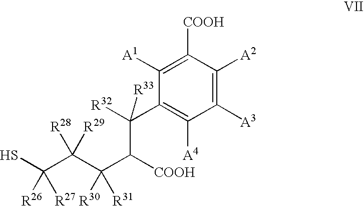

or an enantiomer or a pharmaceutically acceptable equivalent of said compound, wherein:

R²⁶, R²⁷, R²⁸, R²⁹, R³⁰, R³¹, R³² and R³³ are independently hydrogen or C₁-C₃ alkyl;

A¹, A², A³ and A⁴ are independently hydrogen, C₁-C₆ alkyl C₁-C₆ alkoxy, halo, nitro, phenyl, phenoxy, benzyl, benzyloxy or —COOH, or any adjacent two of A², A³ and A⁴ form with the benzene ring a fused 5- or 6-membered carbocyclic or heterocyclic aromatic ring, said heterocyclic aromatic ring containing 1 or 2 oxygen, nitrogen and/or sulfur heteroatom(s).

In one embodiment, R²⁶, R²⁷, R²⁸, R²⁹, R³⁰ R³, R³² and R³³ are independently hydrogen or methyl; and Al, A², A³ and A⁴ are independently hydrogen, C₁-C₄ alkyl, C₁-C₂ alkoxy, halo, nitro, phenyl, phenoxy, benzyloxy, nitro or —COOH.

In another embodiment, any adjacent two of A², A³ and A⁴ form with the benzene ring a fused 5- or 6-membered carbocyclic or heterocyclic aromatic ring, said heterocyclic aromatic ring containing I or 2 oxygen, nitrogen and/or sulfur heteroatom(s).

Formula VIII

Another NAALADase inhibitor is a compound of formula VIII

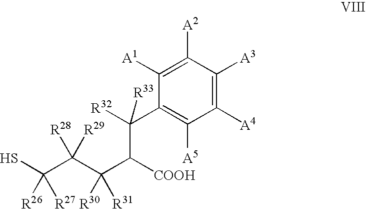

or an enantiomer or a pharmaceutically acceptable equivalent of said compound, wherein:

R²⁶, R²⁷, R²⁸, R²⁹, R³⁰, R³¹, R³² and R³³ are independently hydrogen or C₁-C₃ alkyl; and

A¹, A², A³, A⁴ and A⁵ are independently hydrogen, C₁-C₆ alkyl, C₁-C₆ alkoxy, C₁-C₃ perhaloalkyl, phenyl, phenoxy, benzyl, benzyloxy, hydroxy, halo, cyano, nitro, —SO₂R³⁴, —(C═O)NR³⁴R³⁵, —(C═O)NR³⁴(CH₂)ₙCOOH, —NR³⁴(C═O)R³⁵, —(CH₂)ₙCOOH or —COOH, or any adjacent two of A¹, A², A³, A⁴ and A⁵ form with the benzene ring a fused 5- or 6-membered carbocyclic or heterocyclic aromatic ring, said heterocyclic aromatic ring containing 1 or 2 oxygen, nitrogen and/or sulfur heteroatom(s);

R³⁴ and R³⁵ are independently hydrogen, C₁-C₆ alkyl, phenyl or benzyl; and

n is 1-3.

If A¹, A³ and A⁵ are independently hydrogen, C₁-C₆ alkyl, C₁-C₆ alkoxy, halo, nitro, phenyl, phenoxy, benzyl, benzyloxy or —COOH, then neither A² nor A⁴ are —COOH; and if any adjacent two of A³, A⁴ and A⁵ form with the benzene ring a fused 5- or 6-membered carbocyclic or heterocyclic aromatic ring, said heterocyclic aromatic ring containing 1 or 2 oxygen, nitrogen and/or sulfur heteroatom(s), then A² is not —COOH.

n one embodiment, R²⁶, R²⁷, R²⁸, R²⁹, R³⁰, R³¹, R³² and R³³ are each hydrogen; A, A², A³, A⁴ and A⁵ are independently hydrogen, C₁-C₄ alkyl, C₁-C₂ alkoxy, C₁-C₂ perhaloalkyl, phenyl, phenoxy, hydroxy, halo, cyano, nitro, —SO₂R³⁴, —(C═O)NR³⁴R³⁵, —(C═O)NR³⁴(CH₂)COOH, —NR³⁴(C═O)R³⁵ or —(CH₂)COOH; and R³⁴ and R³⁵ are independently hydrogen, methyl or benzyl.

In another embodiment, any adjacent two of A¹, A², A³, A⁴ and A⁵ form with the benzene ring a fused 5- or 6-membered carbocyclic or heterocyclic aromatic ring, said heterocyclic aromatic ring containing 1 or 2 oxygen, nitrogen and/or sulfur heteroatom(s).

Formula IX

Another NAALADase inhibitor is a compound of formula IX

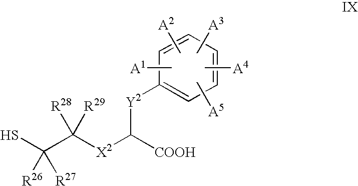

or an enantiomer or a pharmaceutically acceptable equivalent of said compound, wherein:

X² and Y² are independently —CR³⁰R³¹, —O—, —S— or —NR³⁰-, provided that at least one of X² and Y² is/are —CR³⁰R³¹-;

A¹, A², A³, A⁴ and A⁵ are independently hydrogen, C₁-C₉ alkyl, C₂-C₉ alkenyl, C₂-C₉ alkynyl, aryl, heteroaryl, carbocycle, heterocycle, C₁-C₉ alkoxy, C₂-C₉ alkenyloxy, phenoxy, benzyloxy, hydroxy, halo, nitro, cyano, isocyano, —COOR³⁴, —COR³⁴, —NR³⁴R³⁵, —SR³⁴, —SOR³⁴, —SO₂R³⁴, —SO₂(OR³⁴), —(C═O)NR³⁴R³⁵, —(C═O)NR³⁴(CH₂)ₙCOOH, —NR³⁴(C═O)R³⁵ or —(CH₂)ₙCOOH, or any adjacent two of A¹, A², A³, A⁴ and A⁵ form with the benzene ring a fused ring that is saturated or unsaturated, aromatic or non-aromatic, and carbocyclic or heterocyclic, said heterocyclic ring containing 1 or 2 oxygen, nitrogen and/or sulfur heteroatom(s);

n is 1-3; and

R²⁶, R²⁷, R²⁸, R²⁹, R³⁰, R³¹, R³⁴ and R³⁵ are independently hydrogen, C₁-C₉ alkyl, C₂-C₉ alkenyl, C₂-C₉ alkynyl, aryl, heteroaryl, carbocycle or heterocycle; and said alkyl, alkenyl, alkynyl, aryl, heteroaryl, carbocycle, heterocycle, alkoxy, alkenyloxy, phenoxy, benzyloxy, and fused ring are independently unsubstituted or substituted with one or more substituent(s).

If A¹, A² and A³ are each hydrogen, and A⁴ and A⁵ are each —COOH, then A⁴ is ortho to A⁵; and if Y³ is —CR³⁰R³¹, then at least one of A¹, A², A³, A⁴ and A⁵ is/are independently phenoxy, benzyloxy, aryl, heteroaryl, carbocycle or heterocycle that is substituted with one or more substituent(s).

In one embodiment, Y² is —O—, —S— or —NR³⁰-; A¹, A², A³, A⁴ and A⁵ are independently hydrogen, C₁-C₄ alkyl, C₁-C₂ alkoxy, hydroxy, halo, —COOH, —COR³⁴, —NR³⁴(C═O)R³⁵ or —(CH₂)COOH; and R³⁴ and R³⁵ are independently hydrogen or methyl.

In another embodiment, Y² is —CR³⁰R³¹-; A¹, A², A³ and A⁴ are each hydrogen; and A⁵ is phenoxy, benzyloxy, aryl, heteroaryl, carbocycle or heterocycle, wherein said phenoxy and benzyloxy are substituted with —COOH, and said aryl, heteroaryl, carbocycle and heterocycle are independently substituted with one or more substituent(s) selected from cyano and —COOH.

Formula X

Another NAALADase inhibitor is a compound of formula X

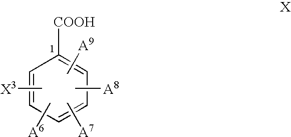

or an enantiomer or a pharmaceutically acceptable equivalent of said compound, wherein:

X³ is —(CR³⁶R³⁷)ₙSH, —O(CR³⁶R³⁷)₂SH, —S(CR³⁶R³⁷)₂SH or —NR(CR³⁶R³⁷)₂SH;

n is 1-3; and

R, R³⁶, R³⁷, A⁶, A⁷, A⁸ and A⁹ are independently hydrogen, C₁-C₉ alkyl, C₂-C₉ alkenyl, C₂-C₉ alkynyl, aryl, heteroaryl, carbocycle, heterocycle, halo, hydroxy, sulfhydryl, nitro, amino, cyano, isocyano, thiocyano, isothiocyano, formamido, thioformamido, sulfo, sulfino, C₁-C₉ alkylsulfonyl, C₁-C₉ alkoxy, C₂-C₉ alkenoxy, phenoxy or benzyloxy, wherein said alkyl, alkenyl, alkynyl, aryl, heteroaryl, carbocycle, heterocycle, alkoxy, alkenoxy, phenoxy and benzyloxy are independently unsubstituted or substituted with one or more substituent(s).

Examples of compounds of formula X include without limitation:

3-(2-mercaptoethyl)-benzoic acid;

3-(mercaptomethyl)-benzoic acid;

2-(mercaptomethyl)-benzoic acid;

5-hydroxy-2-(2-mercaptoethyl)-benzoic acid;

2-(2-mercaptoethyl)-benzoic acid;

5-[(4-carboxyphenyl)methoxy]-2-(2-mercaptoethyl)-benzoic acid;

2-(2-mercaptoethyl)-5-(phenylmethoxy)-benzoic acid;

2-(carboxymethoxy)-6-(2-mercaptoethyl)-benzoic acid;

5-[(3-carboxyphenyl)methoxy]-2-(2-mercaptoethyl)-benzoic acid;

2-(2-mercaptoethyl)-6-(phenylmethoxy)-benzoic acid;

2-[(2-carboxyphenyl)methoxy]-6-(2-mercaptoethyl)-benzoic acid;

2-[(4-carboxyphenyl)methoxy]-6-(2-mercaptoethyl)-benzoic acid;

3-(2-mercaptoethyl)-[1,1′-biphenyl]-2,3′-dicarboxylic acid;

2-(3,3-dimethylbutoxy)-6-(2-mercaptoethyl)-benzoic acid;

2-(2-mercaptoethyl)-6-(2-phenylethoxy)-benzoic acid;

2-[(2-chlorophenyl)methoxy]-6-(2-mercaptoethyl)-benzoic acid;

2-[[3-carboxy-5-(1,1-dimethylethyl)phenyl]methoxy]-6-(2-mercaptoethyl)-benzoic acid;

2-(2-mercaptoethyl)-6-phenoxy-benzoic acid;

2-(2-mercaptoethyl)-6-phenylamino-benzoic acid;

2-(2-mercaptoethyl)-6-(phenylthio)-benzoic acid;

5′-(1,1-dimethylethyl)-3-(2-mercaptoethyl)-[1,1′-biphenyl]-2,3′-dicarboxylic acid;

3-(2-mercaptoethyl)-[1,1′-biphenyl]-2,4′-dicarboxylic acid;

2-[(4-carboxy-2-methoxyphenyl)methoxy]-6-(2-mercaptoethyl)-benzoic acid;

2-[(4-carboxy-3-methoxyphenyl)methoxy]-6-(2-mercaptoethyl)-benzoic acid;

2-[(2-bromo-4-carboxyphenyl)methoxy]-6-(2-mercaptoethyl)-benzoic acid;

2-[(3-bromo-4-carboxyphenyl)methoxy]-6-(2-mercaptoethyl)-benzoic acid;

2-[(4-chlorophenyl)methoxy]-6-(2-mercaptoethyl)-benzoic acid;

2-(biphenyl-2-ylmethoxy)-6-(2-mercaptoethyl)-benzoic acid;

2-[(3-bromo-5-carboxyphenyl)methoxy]-6-(2-mercaptoethyl)-benzoic acid;

2-[(2-bromo-5-carboxyphenyl)methoxy]-6-(2-mercaptoethyl)-benzoic acid;

2-(2-mercaptoethyl)-6-[(4-methoxyphenyl)methoxy]-benzoic acid;

2-(2-mercaptoethyl)-6-[(4-methylphenyl)methoxy]-benzoic acid;

2-[(4-bromo-3-carboxyphenyl)methoxy]-6-(2-mercaptoethyl)-benzoic acid;

2-[(2-carboxy-5-methoxyphenyl)methoxy]-6-(2-mercaptoethyl)-benzoic acid;

5-(mercaptomethyl)-2-(2-phenylethoxy)-benzoic acid;

2-bromo-5-(mercaptomethyl)-benzoic acid;

4-(mercaptomethyl)-[1,1′-biphenyl]-2,3′-dicarboxylic acid;

5-(mercaptomethyl)-2-(phenylmethoxy)-benzoic acid; and

4-bromo-3-(mercaptomethyl)-benzoic acid; and

enantiomers and pharmaceutically acceptable equivalents.

Formula XI

Another NAALADase inhibitor is a compound of formula XI

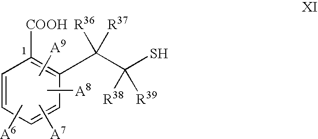

or an enantiomer or a pharmaceutically acceptable equivalent of said compound, wherein:

R³⁷, R³⁸, R³⁹ and R⁴⁰ are independently hydrogen or C₁-C₃ alkyl; and

A⁶, A⁷, A⁸ and A⁹ are independently hydrogen, C₁-C₉ alkyl, C₂-C₉ alkenyl, C₂-C₉ alkynyl, aryl, heteroaryl, carbocycle, heterocycle, halo, hydroxy, sulfhydryl, nitro, amino, cyano, isocyano, thiocyano, isothiocyano, formamido, thioformamido, sulfo, sulfino, C₁-C₉ alkylsulfonyl, C₁-C₉ alkoxy, C₂-C₉ alkenoxy, phenoxy or benzyloxy, wherein said alkyl, alkenyl, alkynyl, aryl, heteroaryl, carbocycle, heterocycle, alkoxy, alkenoxy, phenoxy and benzyloxy are independently unsubstituted or substituted with one or more substituent(s).

In one embodiment, R³⁶, R³⁷, R³⁸ and R³⁹, A⁷, A⁸ and A⁹ are each hydrogen; A⁶ is hydrogen, —(CH₂)ₙ-W¹, or —Y³—(CH₂)ₙ-W¹; n is 0-3; Y³ is O, S or NR⁴⁰; R⁴⁰ is hydrogen or C₁-C₄ alkyl; W¹ is C₁-C₆ alkyl or phenyl, wherein W¹ is unsubstituted or substituted with C₁-C₄ alkyl, C₁-C₄ alkoxy, carboxy or halo.

Formula XII

Another NAALADase inhibitor is a compound of formula XII

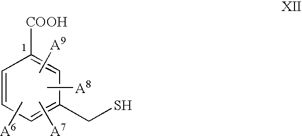

or an enantiomer or a pharmaceutically acceptable equivalent of said compound, wherein:

A⁶, A⁷, A⁸ and A⁹ are independently hydrogen, C₁-C₉ alkyl, C₂-C₉ alkenyl, C₂-C₉ alkynyl, aryl, heteroaryl, carbocycle, heterocycle, halo, hydroxy, sulfhydryl, nitro, amino, cyano, isocyano, thiocyano, isothiocyano, formamido, thioformamido, sulfo, sulfino, C₁-C₉ alkylsulfonyl, C₁-C₉ alkoxy, C₂-C₉ alkenoxy, phenoxy or benzyloxy, wherein said alkyl, alkenyl, alkynyl, aryl, heteroaryl, carbocycle, heterocycle, alkoxy, alkenoxy, phenoxy and benzyloxy are independently unsubstituted or substituted with one or more substituent(s).

At least one of A⁶, A⁷, A⁸ and A⁹ is/are not hydrogen; and if A⁶ is chloro, fluoro, amino or thiomethyl, then at least one of A⁷, A⁸ and A⁹ is/are not hydrogen.

In one embodiment, A⁷, A⁸ and A⁹ are each hydrogen; A⁶ is —(CH₂)ₙ-Ar² or —Y³—(CH₂)ₙ-Ar²; n is 0-3; Y³ is O, S or NR⁴¹; R⁴¹ is hydrogen or C₁-C₄ alkyl; and Ar² is phenyl, wherein Ar² is unsubstituted or substituted with C₁-C₄ alkyl, carboxy or halo.

Formula XIII

Another NAALADase inhibitor is a compound of formula XIII

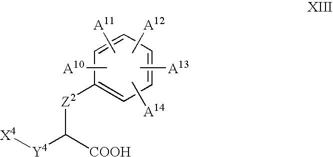

or an enantiomer pharmaceutically acceptable equivalent of said compound, wherein:

X⁴ is —(CO)NHOH or —N(OH)COH;

Y⁴ is a bond or a divalent linking group having from 1 to 9 carbon atom(s) and from 0 to 5 heteroatom(s) independently selected from oxygen, sulfur and nitrogen;

Z² is —CR⁴¹R⁴²-, —NR⁴¹-, —O— or —S—;

A¹⁰, A¹¹, A¹², A¹³ and A¹⁴ are independently hydrogen, C₁-C₉ alkyl, C₂-C₉ alkenyl, C₂-C₉ alkynyl, aryl, heteroaryl, carbocycle, heterocycle, C₁-C₉ alkoxy, C₂-C₉ alkenyloxy, phenoxy, benzyloxy, hydroxy, halo, nitro, cyano, isocyano, —COOR⁴³, —COR⁴³, —NR⁴³R⁴⁴, —SR⁴³, —SOR⁴³, —SO₂R⁴³, —SO₂(OR⁴³), —(CO)NR⁴³R⁴³, —(CO)NR⁴³(CH₂)ₙCOOH, —NR⁴³(CO)R⁴⁴ or —(CH₂)ₙCOOH, or any adjacent two of A¹⁰, A¹¹, A¹² and A¹³ form with the benzene ring a fused ring that is saturated or unsaturated, aromatic or non-aromatic, and carbocyclic or heterocyclic, said heterocyclic ring containing 1 or 2 oxygen, nitrogen and/or sulfur heteroatom(s);

n is 1-3;

R⁴¹, R⁴², R⁴³ and R⁴⁴ are independently hydrogen, C₁-C₉ alkyl, C₂-C₉ alkenyl, C₂-C₉ alkynyl, aryl, heteroaryl, carbocycle or heterocycle; and

said alkyl, alkenyl, alkynyl, aryl, heteroaryl, carbocycle, heterocycle, alkoxy, alkenyloxy, phenoxy, benzyloxy, and fused ring are independently unsubstituted or substituted with one or more substituent(s).

In one embodiment, Y⁴ is —(CR⁴⁵R⁴⁶)ₚ-W²-(CR⁴⁷R⁴⁸)_{q}-; W² is —CR⁴⁹R⁵⁰-, —NR⁴⁹-, —O—, —S— or —SO₂—; p and q are independently 0-4, provided that when q is 0 and W² is —NR⁴⁹-, —O—, —S— or —SO₂—, then Z² is —CR⁴¹R⁴²-; R⁴⁵, R⁴⁶, R⁴⁷, R⁴⁸, R⁴⁹ and R⁵⁰ are independently hydrogen, C₁-C₉ alkyl, C₂-C₉ alkenyl, C₂-C₉ alkynyl, aryl, heteroaryl, carbocycle, heterocycle, halo, hydroxy, sulfhydryl, nitro, amino, cyano, isocyano, thiocyano, isothiocyano, formamido, thioformamido, sulfo, sulfino, C₁-C₉ alkoxy, C₂-C₉ alkenoxy, phenoxy or benzyloxy, wherein said alkyl, alkenyl, alkynyl, aryl, heteroaryl, carbocycle, heterocycle, alkoxy, alkenyloxy, phenoxy and benzyloxy are independently unsubstituted or substituted with one or more substituent(s); and A¹⁰, A¹¹ and A¹² are each hydrogen.

In another embodiment, Y⁴ is —(CR⁴⁵R⁴⁶)ₚ-W²-(CR⁴⁷R⁴⁸)_{q}-; W² is —CR⁴⁹R⁵⁰-; p is 0-4; q is 0; R⁴⁵, R⁴⁶, R⁴⁷, R⁴⁸, R⁴⁹ and R⁵⁰ are each hydrogen; A¹⁰, A¹¹ and A¹² are each hydrogen; A¹³ is hydrogen, —COOR⁴³, C₁-C₄ alkyl, C₂-C₄ alkenyl or C₂-C₄ alkynyl; and A¹⁴ is —COOR⁴³.

In another embodiment, Y⁴ is —(CR⁴⁵R⁴⁶)ₚ-W²-(CR⁴⁷R⁴⁸)_{q}-; W² is —S—; p and q are independently 1-4; R⁴⁵, R⁴⁶, R⁴⁷, R⁴⁸, R⁴⁹ and R⁵⁰ are independently hydrogen, C₁-C₄ alkyl, C₂-C₄ alkenyl, C₂-C₄ alkynyl or phenyl; A¹⁰, A¹¹ and A¹² are each hydrogen; A¹³ is hydrogen, C₁-C₄ alkyl, C₂-C₄ alkenyl, C₂-C₄ alkynyl, phenyl, benzyl, phenoxy, benzyloxy or halo, wherein said alkyl, alkenyl, alkynyl, phenyl, benzyl, phenoxy and benzyloxy are independently unsubstituted or substituted with carobxy; and A¹⁴ is —COOH.

In another embodiment, Y⁴ is —(CR⁴⁵R⁴⁶)ₚ-W²-(CR⁴⁷R⁴⁸)_{q}-; W² is —CR⁴⁹R⁵⁰-, —NR⁴⁹-, —O—, —S— or —SO₂—; p and q are independently 0-4, provided that when q is 0 and W² is —NR⁴⁹-, —O—, —S— or —SO₂—, then Z² is —CR⁴¹R⁴²-; R⁴⁵, R⁴⁶, R⁴⁷, R⁴⁸, R⁴⁹ and R⁵⁰ are independently hydrogen, C₁-C₉ alkyl, C₂-C₉ alkenyl, C₂-C₉ alkynyl, aryl, heteroaryl, carbocycle, heterocycle, halo, hydroxy, sulfhydryl, nitro, amino, cyano, isocyano, thiocyano, isothiocyano, formamido, thioformamido, sulfo, sulfino, C₁-C₉ alkoxy, C₂-C₉ alkenoxy, phenoxy or benzyloxy, wherein said alkyl, alkenyl, alkynyl, aryl, heteroaryl, carbocycle, heterocycle, alkoxy, alkenyloxy, phenoxy and benzyloxy are independently unsubstituted or substituted with one or more substituent(s); A¹⁰, A¹¹ and A¹² are each hydrogen; A¹³ is hydrogen; and A¹⁴ is benzyl or carboxybenzyl.

Formula XIV

Another NAALADase inhibitor is a compound of formula XIV

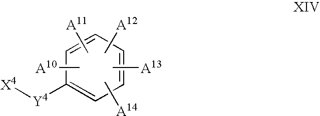

or an enantiomer or a pharmaceutically acceptable equivalent of said compound, wherein:

X⁴ is —(CO)NHOH or —N(OH)COH;

Y⁴ is a bond or a divalent linking group having from 1 to 9 carbon atom(s) and from 0 to 5 heteroatom(s) independently selected from oxygen, sulfur and nitrogen;

A¹⁰, A¹¹, A¹², A¹³ and A¹⁴ are independently hydrogen, C₁-C₉ alkyl, C₂-C₉ alkenyl, C₂-C₉ alkynyl, aryl, heteroaryl, carbocycle, heterocycle, C₁-C₉ alkoxy, C₂-C₉ alkenyloxy, phenoxy, benzyloxy, hydroxy, halo, nitro, cyano, isocyano, —COOR⁴³, COR⁴³, —NR⁴³R⁴⁴, —SR⁴³, —SOR⁴³, —SO₂R⁴³, —SO₂(OR⁴³), —(CO)NR⁴³R⁴⁴, —(CO)NR⁴³(CH₂)ₙCOOH, —NR⁴³(CO)R⁴⁴ or —(CH₂)ₙCOOH, or any adjacent two of A¹⁰, A¹¹, A¹² and A¹³ form with the benzene ring a fused ring that is saturated or unsaturated, aromatic or non-aromatic, and carbocyclic or heterocyclic, said heterocyclic ring containing 1 or 2 oxygen, nitrogen and/or sulfur heteroatom(s);

n is 1-3;

R⁴³ and R⁴⁴ are independently hydrogen, C₁-C₉ alkyl, C₂-C₉ alkenyl, C₂-C₉ alkynyl, aryl, heteroaryl, carbocycle or heterocycle; and

said alkyl, alkenyl, alkynyl, aryl, heteroaryl, carbocycle, heterocycle, alkoxy, alkenyloxy, phenoxy, benzyloxy, and fused ring are independently unsubstituted or substituted with one or more substituent(s).

In one embodiment, Y⁴ is a bond or —(CR⁴⁵R⁴⁶)ₚ-W²-(CR⁴⁷R⁴⁸)_{q}-; W² is —CR⁴⁹R⁵⁰-, —NR⁴⁹-, —O—, —S— or —SO₂—; p and q are independently 0-4; R⁴⁵, R⁴⁶, R⁴⁷, R⁴⁸, R⁴⁹ and R⁵⁰ are independently hydrogen, C₁-C₉ alkyl, C₂-C₉ alkenyl, C₂-C₉ alkynyl, aryl, heteroaryl, carbocycle, heterocycle, halo, hydroxy, sulfhydryl, nitro, amino, cyano, isocyano, thiocyano, isothiocyano, formamido, thioformamido, sulfo, sulfino, C₁-C₉ alkoxy, C₂-C₉ alkenoxy, phenoxy or benzyloxy, wherein said alkyl, alkenyl, alkynyl, aryl, heteroaryl, carbocycle, heterocycle, alkoxy, alkenyloxy, phenoxy and benzyloxy are independently unsubstituted or substituted with one or more substituent(s); and A¹⁰, A¹¹ and A¹² are each hydrogen.

In another embodiment, Y⁴ is a bond; A¹⁰, A¹¹ and A¹² are each hydrogen; A¹³ is hydroxy, phenoxy, benzyloxy, —COOR⁴³ or —(CO)NHR⁴⁴; A¹⁴ is —COOR⁴³; R⁴³ is hydrogen, C₁-C₄ alkyl, C₂-C₄ alkenyl or C₂-C₄ alkynyl; R⁴⁴ is benzyl; and said benzyl, phenoxy and benzyloxy are independently unsubstituted or substituted with —-COOR⁴³.

In another embodiment, Y⁴ is —(CR⁴⁵R⁴⁶)ₚ-W²-(CR⁴⁷R⁴⁸)_{q}-; W² is —O— or —S—; R⁴⁵, R⁴⁶, R⁴⁷ and R⁴⁸ are each hydrogen; A¹⁰, A¹¹ and A¹² are each hydrogen; and A¹³ is hydrogen, —COOH, phenyl or benzyloxy, wherein said phenyl and benzyloxy are independently unsubstituted or substituted with —COOR⁴³; and A¹⁴ is —COOR⁴³.

In another embodiment, Y⁴ is a bond or —(CR⁴⁵R⁴⁶)ₚ-W²-(CR⁴⁷R⁴⁸)_{q}-; W² is —CR⁴⁹R⁵⁰-, —NR⁴⁹-, —O—, —S— or —SO₂—; p and q are independently 0-4; R⁴⁵, R⁴⁶, R⁴⁷, R⁴⁸, R⁴⁹ and R⁵⁰ are independently hydrogen, C₁-C₉ alkyl, C₂-C₉ alkenyl, C₂-C₉ alkynyl, aryl, heteroaryl, carbocycle, heterocycle, halo, hydroxy, sulfhydryl, nitro, amino, cyano, isocyano, thiocyano, isothiocyano, formamido, thioformamido, sulfo, sulfino, C₁-C₉ alkoxy, C₂-C₉ alkenoxy, phenoxy or benzyloxy, wherein said alkyl, alkenyl, alkynyl, aryl, heteroaryl, carbocycle, heterocycle, alkoxy, alkenyloxy, phenoxy and benzyloxy are independently unsubstituted or substituted with one or more substituent(s); A¹⁻, A¹¹ and A¹² are each hydrogen; A¹³ is hydrogen, nitro or C₁-C₄ alkoxy; A¹⁴ is hydroxy, phenoxy, benzyloxy, benzoyl or C₁-C₄ alkoxy, wherein said phenoxy, benzyloxy, benzoyl and alkoxy are independently unsubstituted or substituted with one or more substituent(s).

Formula XV

Another NAALADase inhibitor is a compound of formula XV

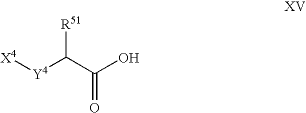

or an enantiomer or a pharmaceutically acceptable equivalent of said compound, wherein:

X⁴ is —(CO)NHOH or —N(OH)COH;

Y⁴ is a bond or a divalent linking group having from 1 to 9 carbon atom(s) and from 0 to 5 heteroatom(s) independently selected from oxygen, sulfur and nitrogen; and

R⁵¹ is hydrogen, C₁-C₉ alkyl, C₂-C₉ alkenyl, C₂-C₉ alkynyl, C₁-C₉ alkoxy or C₂-C₉ alkenoxy, wherein said alkyl, alkenyl, alkynyl, alkoxy and alkenoxy are independently unsubstituted or substituted with one or more substituent(s); provided that when Y is methylene, amine or oxygen, then R⁵¹ is not carboxyethyl.

In one embodiment, Y⁴ is —(CR⁴⁵R⁴⁶)ₚ-W²-(CR⁴⁷R⁴⁸)_{q}-; W² is —CR⁴⁹R⁵⁰-, —NR⁴⁹-, —O—, —S— or —SO₂—; p and q are independently 0-4; and R⁴⁵, R⁴⁶, R⁴⁷, R⁴⁸, R⁴⁹ and R⁵⁰ are independently hydrogen, C₁-C₉ alkyl, C₂-C₉ alkenyl, C₂-C₉ alkynyl, aryl, heteroaryl, carbocycle, heterocycle, halo, hydroxy, sulfhydryl, nitro, amino, cyano, isocyano, thiocyano, isothiocyano, formamido, thioformamido, sulfo, sulfino, C₁-C₉ alkoxy, C₂-C₉ alkenoxy, phenoxy or benzyloxy, wherein said alkyl, alkenyl, alkynyl, aryl, heteroaryl, carbocycle, heterocycle, alkoxy, alkenyloxy, phenoxy and benzyloxy are independently unsubstituted or substituted with one or more substituent(s).

In another embodiment, Y⁴ is —(CR⁴⁵R⁴⁶)ₚ-W²-(CR⁴⁷R⁴⁸)_{q}-; W² is -CR⁴⁹R⁵⁰- or —S—; p is 0-1; q is 0-3; and R⁴⁵, R⁴⁶, R⁴⁷, R⁴⁸, R⁴⁹ and R⁵⁰ are each hydrogen.

Examples of compounds of formulas XIII-XV are set forth below in TABLE I.

TABLE I

| Structure | Name |
|---|---|
| 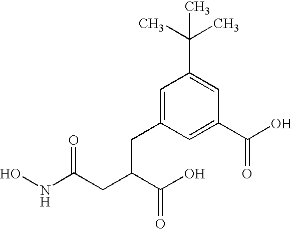 | 3-tert-Butyl-5-(2-carboxy-3- hydroxycarbamoyl-propyl)-benzoic acid |
| 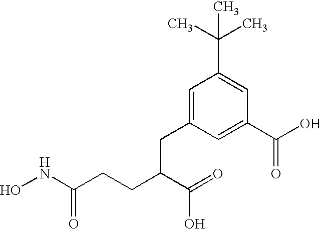 | 3-tert-Butyl-5-(2-carboxy-4- hydroxycarbamoyl-butyl)-benzoic acid |
| 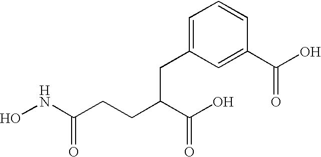 | 3-(2-Carboxy-4-hydroxycarbamoyl- butyl)-benzoic acid |
| 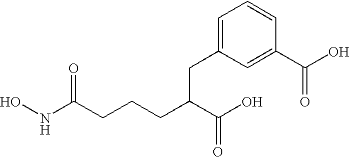 | 3-(2-Carboxy-5-hydroxycarbamoyl- pentyl)-benzoic acid |
| 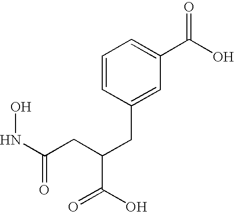 | 3-(2-Carboxy-3-hydroxycarbamoyl- propyl)-benzoic acid |
| 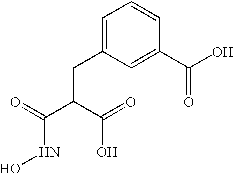 | 3-(2-Carboxy-2-hydroxycarbamoyl- ethyl)-benzoic acid |
| 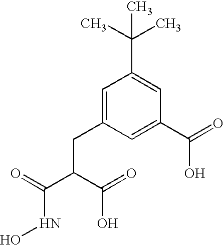 | 3-tert-Butyl-5-(2-carboxy-2- hydroxycarbamoyl-ethyl)-benzoic acid |
| 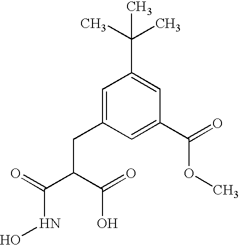 | 3-tert-Butyl-5-(2-carboxy-2- hydroxycarbamoyl-ethyl)-benzoic acid methyl ester |
| 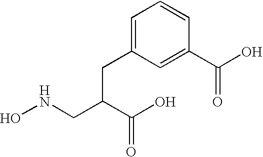 | 3-(2-Carboxy-3-hydroxyamino- propyl)-benzoic acid |
| 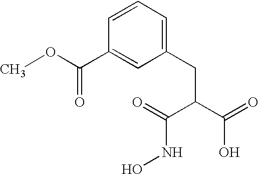 | 3-(2-Carboxy-2-hydroxycarbamoyl- ethyl)-benzoic acid methyl ester |
| 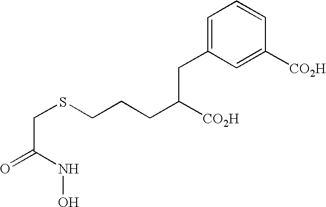 | 3-(2-Carboxy-5- hydroxycarbamoylmethyl- sulfanylpentyl)-benzoic acid |
| 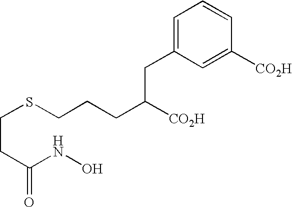 | 3-[2-Carboxy-5-(2-hydroxycarbamoyl- ethylsulfanyl)-pentyl]-benzoic acid |
| 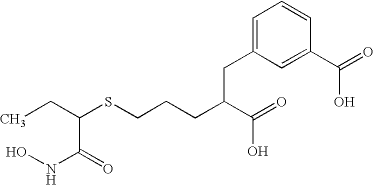 | 3-[2-Carboxy-5-(1-hydroxycarbamoyl- propylsulfanyl)-pentyl]-benzoic acid |
| 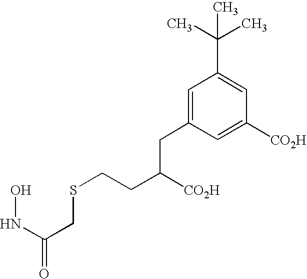 | 3-tert-Butyl-5-(2-carboxy-4- hydroxycarbamoylmethyl- sulfanylbutyl)-benzoic acid |
| 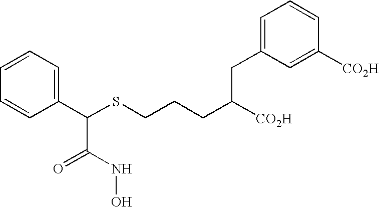 | 3-[2-Carboxy-5-(hydroxycarbamoyl- phenyl-methylsulfanyl)-pentyl]- benzoic acid |
| 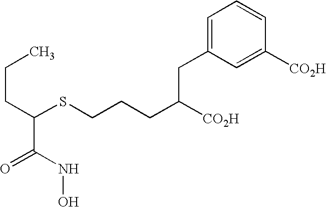 | 3-[2-Carboxy-5-(1-hydroxycarbamoyl- butylsulfanyl)-pentyl]-benzoic acid |
| 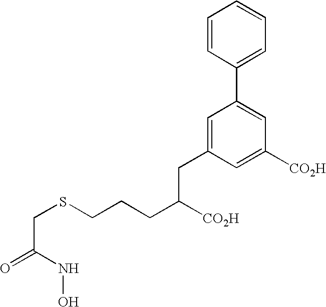 | 5-(2-Carboxy-5- hydroxycarbamoylmethyl- sulfanylpentyl)-biphenyl-3-carboxylic acid |
| 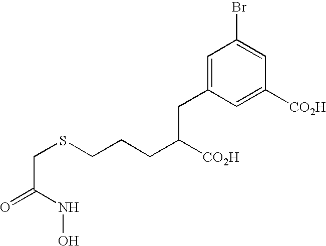 | 3-Bromo-5-(2-carboxy-5- hydroxycarbamoylmethyl- sulfanylpentyl)-benzoic acid |
| 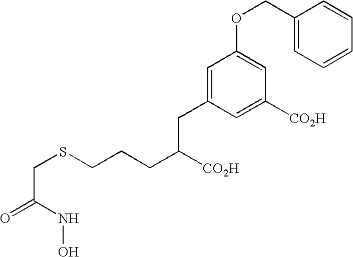 | 3-Benzyloxy-5-(2-carboxy-5- hydroxycarbamoylmethyl- sulfanylpentyl)-benzoic acid |
| 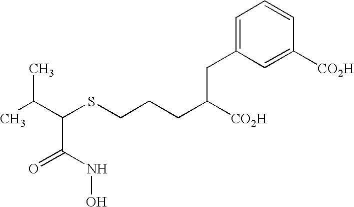 | 3-[2-Carboxy-5-(1-hydroxycarbamoyl- 2-methyl-propylsulfanyl)-pentyl]- benzoic acid |
| 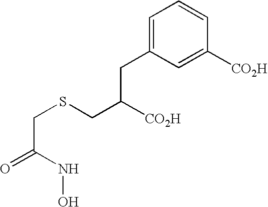 | 3-(2-Carboxy-3- hydroxycarbamoylmethyl- sulfanylpropyl)-benzoic acid |
| 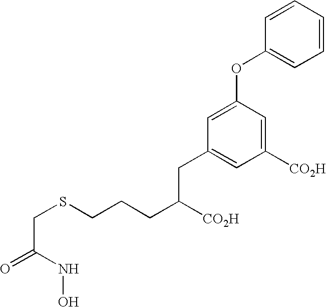 | 3-(2-Carboxy-5- hydroxycarbamoylmethyl- sulfanylpentyl)-5-phenoxy-benzoic acid |
| 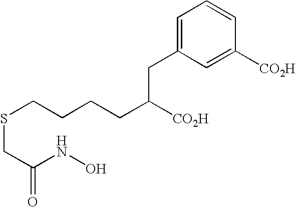 | 3-(2-Carboxy-6- hydroxycarbamoylmethyl- sulfanylhexyl)-benzoic acid |
| 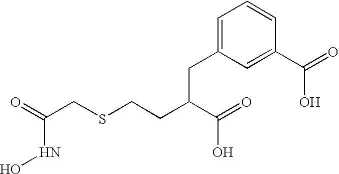 | 3-(2-Carboxy-4- hydroxycarbamoylmethyl- sulfanylbutyl)-benzoic acid |
| 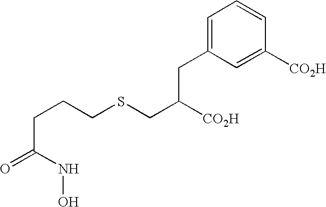 | 3-[2-Carboxy-3-(3-hydroxycarbamoyl- propylsulfanyl)-propyl]-benzoic acid |
| 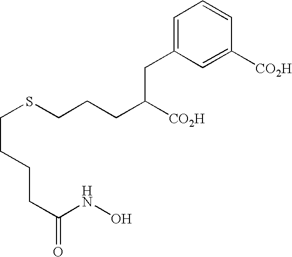 | 3-[2-Carboxy-5-(4-hydroxycarbamoyl- butylsulfanyl)-pentyl]-benzoic acid |
| 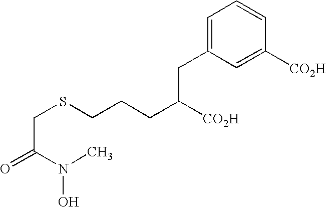 | 3-{2-Carboxy-5-[(hydroxy-methyl- carbamoyl)-methylsulfanyl]-pentyl}- benzoic acid |
| 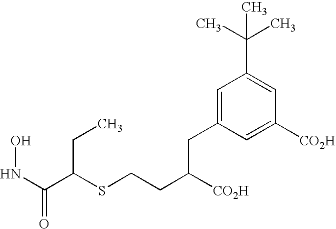 | 3-tert-Butyl-5-[2-carboxy-4-(1- hydroxycarbamoyl-propylsulfanyl)- butyl]-benzoic acid |
| 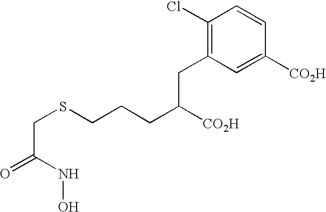 | 3-(2-Carboxy-5- hydroxycarbamoylmethyl- sulfanylpentyl)-4-chloro-benzoic acid |
| 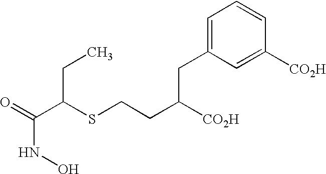 | 3-[2-Carboxy-4-(1-hydroxycarbamoyl- propylsulfanyl)-butyl]-benzoic acid |
| 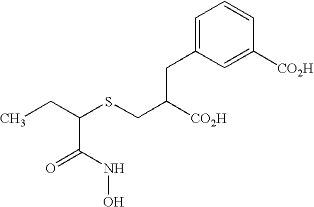 | 3-[2-Carboxy-3-(1-hydroxycarbamoyl- propylsulfanyl)-propyl]-benzoic acid |
| 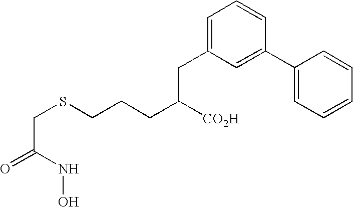 | 2-Biphenyl-3-ylmethyl-5- hydroxycarbamoylmethylsulfanyl- pentanoic acid |
| 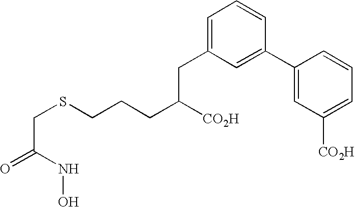 | 3′-(2-Carboxy-5- hydroxycarbamoylmethylsulfanyl- pentyl)-biphenyl-3-carboxylic acid |
| 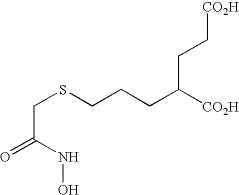 | 2-(3-Hydroxycarbamoyl- methylsulfanylpropyl)-pentanedioic acid |
| 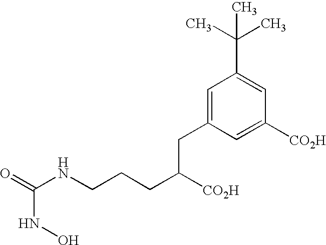 | 3(2-Carboxy-5-{[(hydroxy- amino)carbonyl]amino}-pentyl)-5-tert- butylbenzoic acid |
| 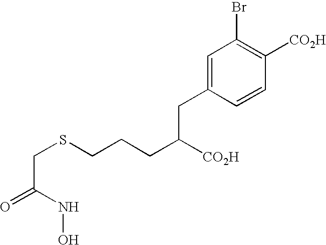 | 2-Bromo-4-(2-carboxy-5- hydroxycarbamoylmethyl- sulfanylpentyl)-benzoic acid |
| 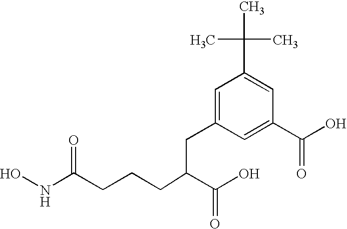 | |
| 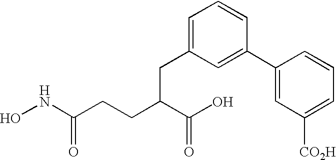 | |
| 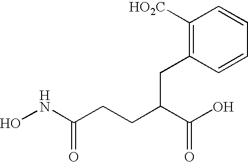 | |
| 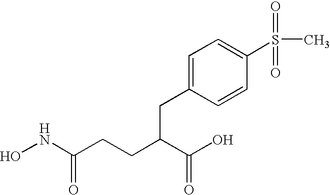 | |
| 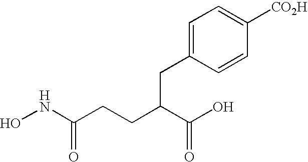 | 4-[2-carboxy-5-(hydroxyamino)-5- oxopentyl]benzoic acid |
| 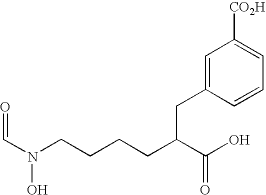 | |
| 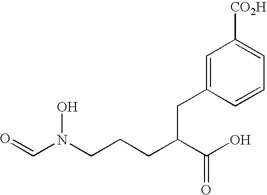 | |
| 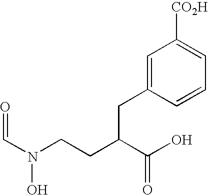 | |
| 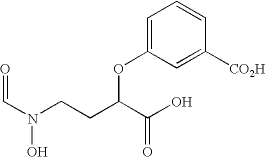 | |
| 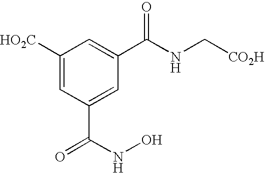 | |
| 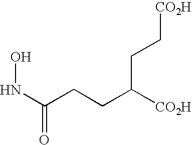 | |
| 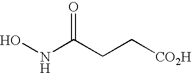 | |
| 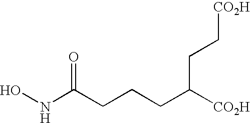 | |
| 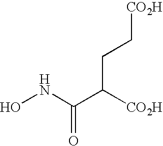 | |
| 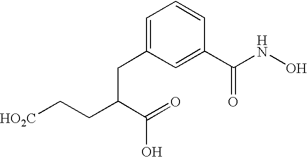 | |

Other NAALADase Inhibitors

Other NAALADase inhibitors are described in International Publication No. WO 01/14390 and U.S. Pat. No. 6,348,464, the entire contents of which publication and patent are herein incorporated by reference as though set forth herein in full.

Possible substituents of the compounds of formulas I-XV include, without limitation, C₁-C₆ alkyl, C₂-C₆ alkenyl, C₂-C₆ alkynyl, C₁-C₆ alkoxy, C₂-C₆ alkenyloxy, phenoxy, benzyloxy, hydroxy, carboxy, hydroperoxy, carbamido, carbamoyl, carbamyl, carbonyl, carbozoyl, amino, hydroxyamino, formamido, formyl, guanyl, cyano, cyanoamino, isocyano, isocyanato, diazo, azido, hydrazino, triazano, nitrilo, nitro, nitroso, isonitroso, nitrosamino, imino, nitrosimino, oxo, C₁-C₆ alkylthio, sulfamino, sulfamoyl, sulfeno, sulfhydryl, sulfinyl, sulfo, sulfonyl, thiocarboxy, thiocyano, isothiocyano, thioformamido, halo, haloalkyl, chlorosyl, chloryl, perchloryl, trifluoromethyl, iodosyl, iodyl, phosphino, phosphinyl, phospho, phosphono, arsino, selanyl, disilanyl, siloxy, silyl, silylene and carbocyclic and heterocyclic moieties.

Carbocyclic moieties include alicyclic and aromatic structures. Examples of carbocyclic and heterocyclic moieties include, without limitation, phenyl, benzyl, naphthyl, indenyl, azulenyl, fluorenyl, anthracenyl, indolyl, isoindolyl, indolinyl, benzofuranyl, benzothiophenyl, indazolyl, benzimidazolyl, benzthiazolyl, tetrahydrofuranyl, tetrahydropyranyl, pyridyl, pyrrolyl, pyrrolidinyl, pyridinyl, pyrimidinyl, purinyl, quinolinyl, isoquinolinyl, tetrahydroquinolinyl, quinolizinyl, furyl, thiophenyl, imidazolyl, oxazolyl, benzoxazolyl, thiazolyl, isoxazolyl, isotriazolyl, oxadiazolyl, triazolyl, thiadiazolyl, pyridazinyl, pyrimidinyl, pyrazinyl, triazinyl, trithianyl, indolizinyl, pyrazolyl, pyrazolinyl, pyrazolidinyl, thienyl, tetrahydroisoquinolinyl, cinnolinyl, phthalazinyl, quinazolinyl, quinoxalinyl, naphthyridinyl, pteridinyl, carbazolyl, acridinyl, phenazinyl, phenothiazinyl, and phenoxazinyl.

All variables of formulas I-XV are independently selected at each occurrence. For example, formula II may have two different CR¹⁰R¹¹ moieties when X is a moiety of formula III and n is 2, with the first CR¹⁰R¹¹ moiety being CH₂, and the second CR¹⁰OR¹¹ moiety being CH(CH₃).

The compounds of formulas I-XV may possess one or more asymmetric carbon center(s) and, thus, may be capable of existing in the form of optical isomers as well as in the form of racemic or non-racemic mixtures of optical isomers. The optical isomers can be obtained by resolution of the racemic mixtures according to conventional processes well known in the art, for example by formation of diastereoisomeric salts by treatment with an optically active acid or base, and then separation of the mixture of diastereoisomers by crystallization followed by liberation of the optically active bases from these salts. Examples of optically active acids are tartaric, diacetyltartaric, dibenzoyltartaric, ditoluoyltartaric and camphorsulfonic acid. A different process for separation of optical isomers involves the use of a chiral chromatography column optimally chosen to maximize the separation of the enantiomers. Still another available method involves synthesis of covalent diastereoisomeric molecules, for example, esters, amides, acetals, ketals, and the like, by reacting compounds used in the inventive methods and pharmaceutical compositions with an optically active acid in an activated form, an optically active diol or an optically active isocyanate. The synthesized diastereoisomers can be separated by conventional means such as chromatography, distillation, crystallization or sublimation, and then hydrolyzed to deliver the enantiomerically pure compound. In some cases hydrolysis to the parent optically active drug is not necessary prior to dosing the patient since the compound can behave as a prodrug. The optically active compounds can likewise be obtained by utilizing optically active starting materials.

It is understood that the compounds of formulas I-XV encompass optical isomers as well as racemic and non-racemic mixtures.

Synthesis of NAALADase Inhibitors

Some of the NAALADase inhibitors used in the inventive methods and pharmaceutical compositions can be readily prepared by standard techniques of organic chemistry, utilizing the general synthetic pathways and examples depicted in U.S. Pat. Nos. 5,672,592, 5,795,877, 5,863,536, 5,880,112, 5,902,817, 5,962,521, 5,968,915, 6,025,344, 6,025,345, 6,028,216, 6,046,180, 6,054,444, 6,071,965, 6,121,252, 6,265,609, 6,348,464, 6,452,044, 6,458,775, 6,586,623, and International Publications Nos. WO 01/14390, WO 02/096866, WO 03/057670 and WO 02/092553, the entire contents of which patents and publications are herein incorporated by reference, as though set forth herein in full.

Other NAALADase inhibitors may be available from commercial suppliers or can be readily prepared by an ordinarily skilled artisan using standard techniques such as those disclosed in U.S. Pat. No. 5,859,046, the entire contents of which reference are herein incorporated by reference as though set forth herein in full.

Yet other NAALADase inhibitors can be readily prepared by standard techniques of organic chemistry, utilizing the general synthetic pathways depicted below in SCHEMES I-XIV.

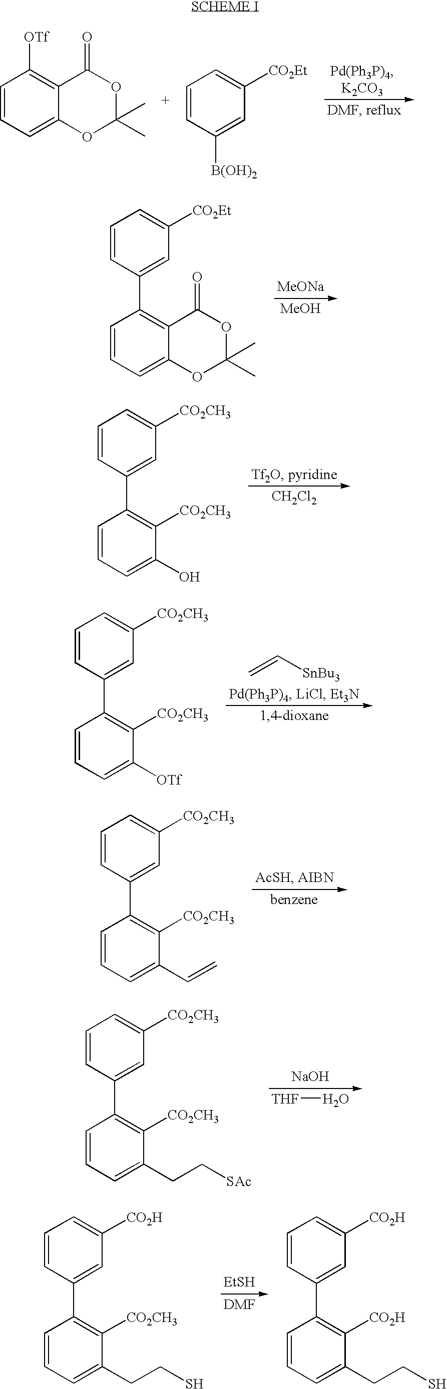

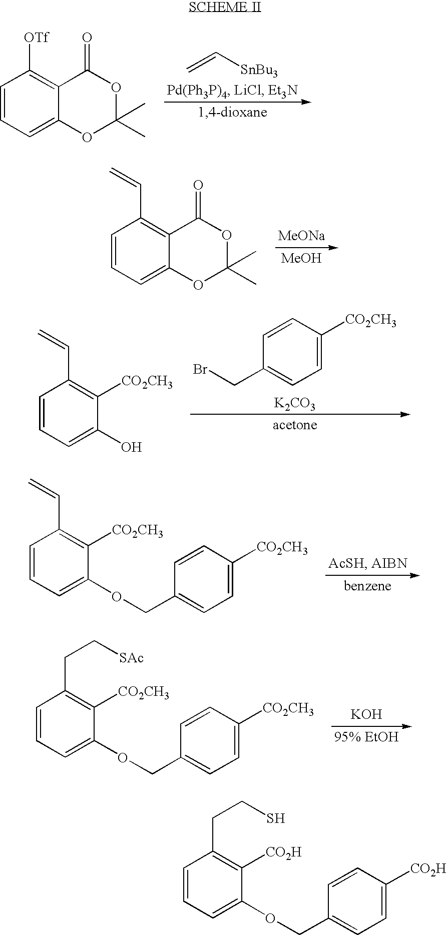

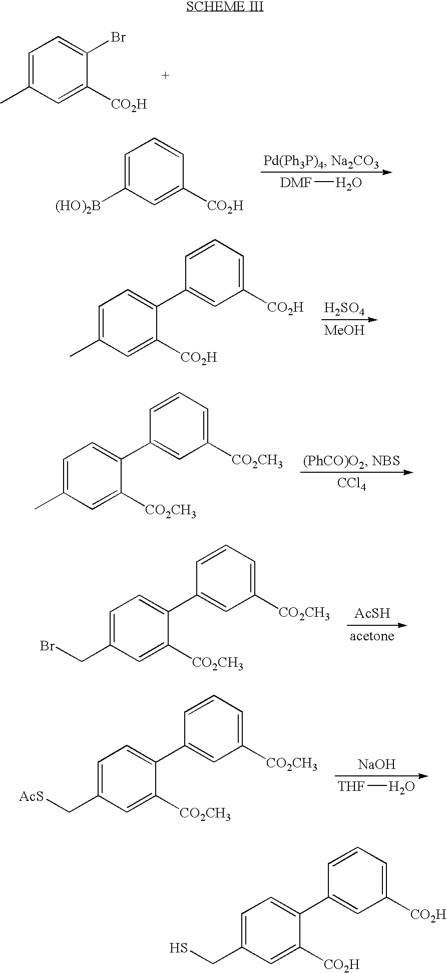

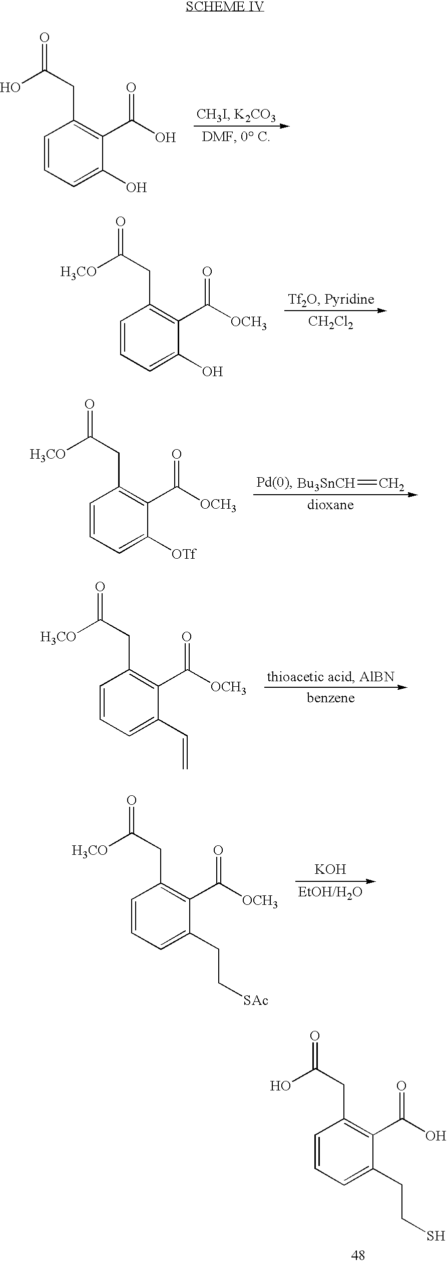

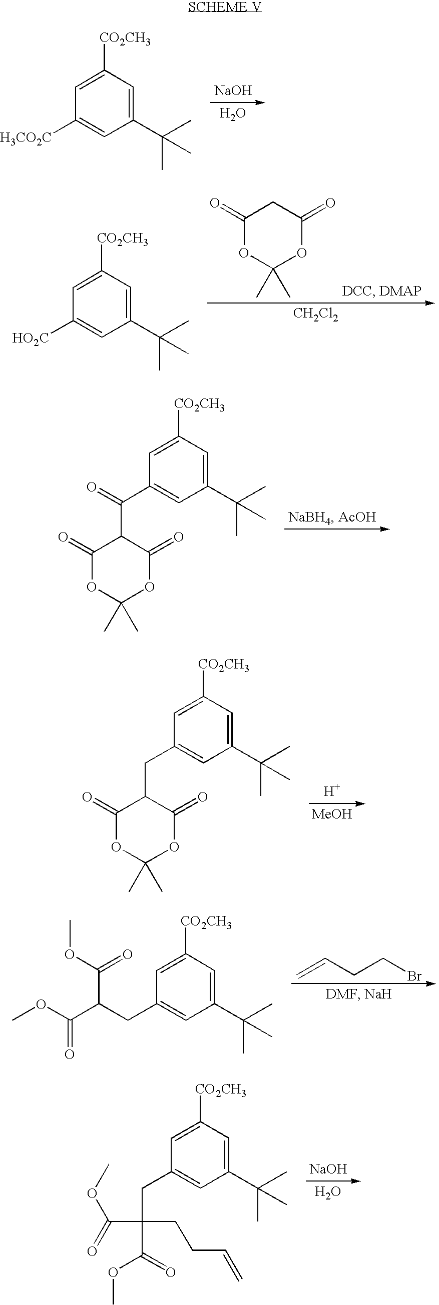

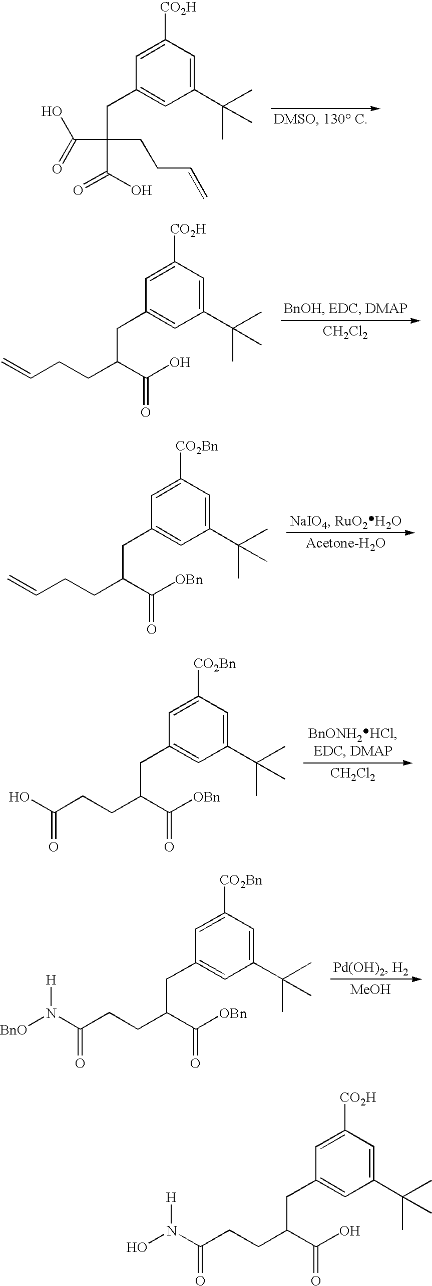

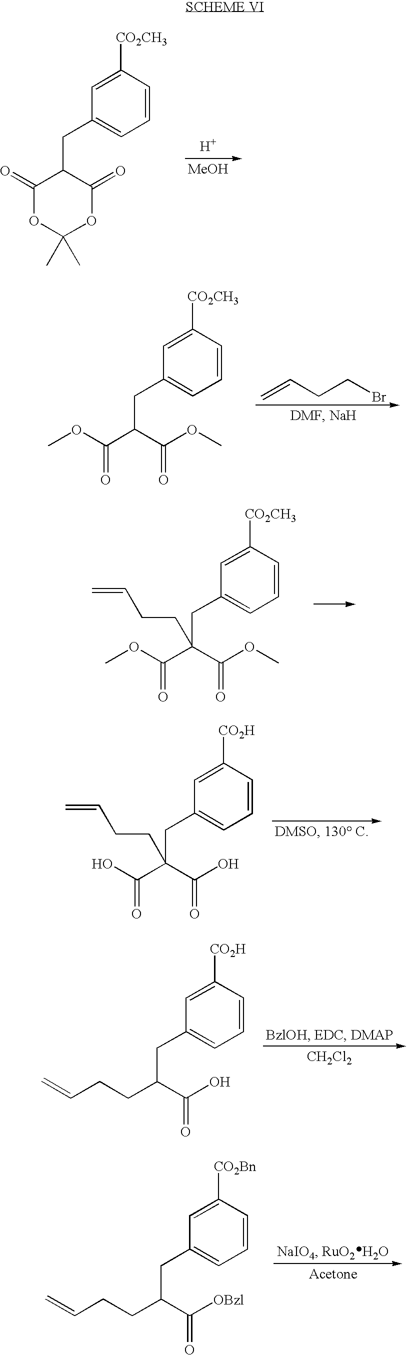

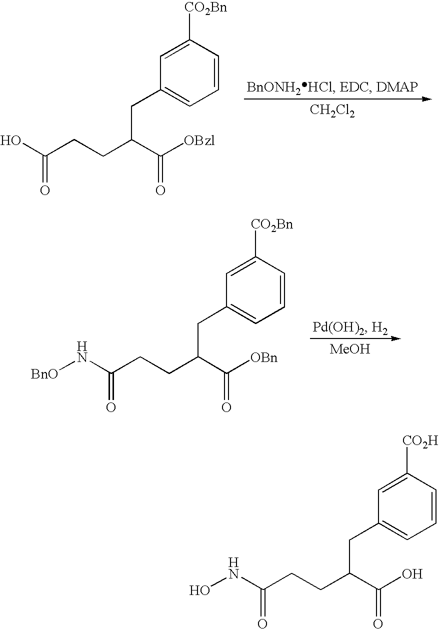

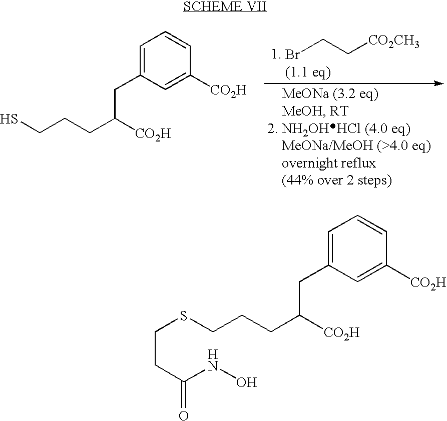

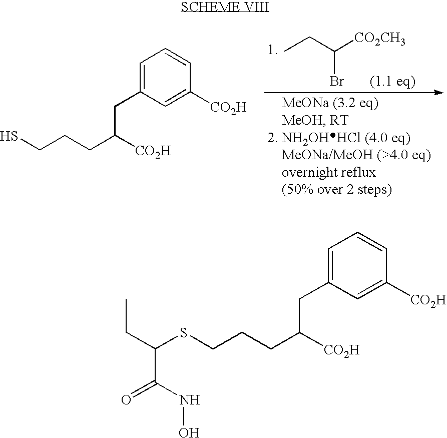

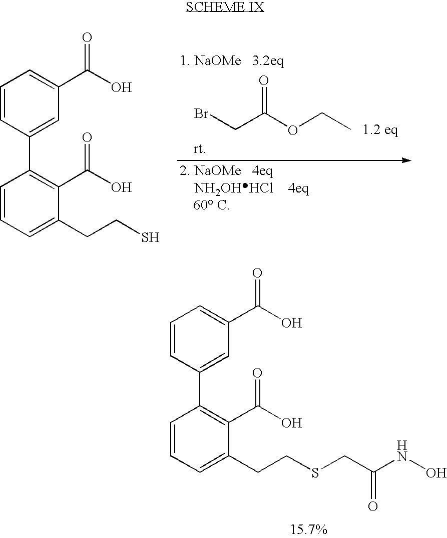

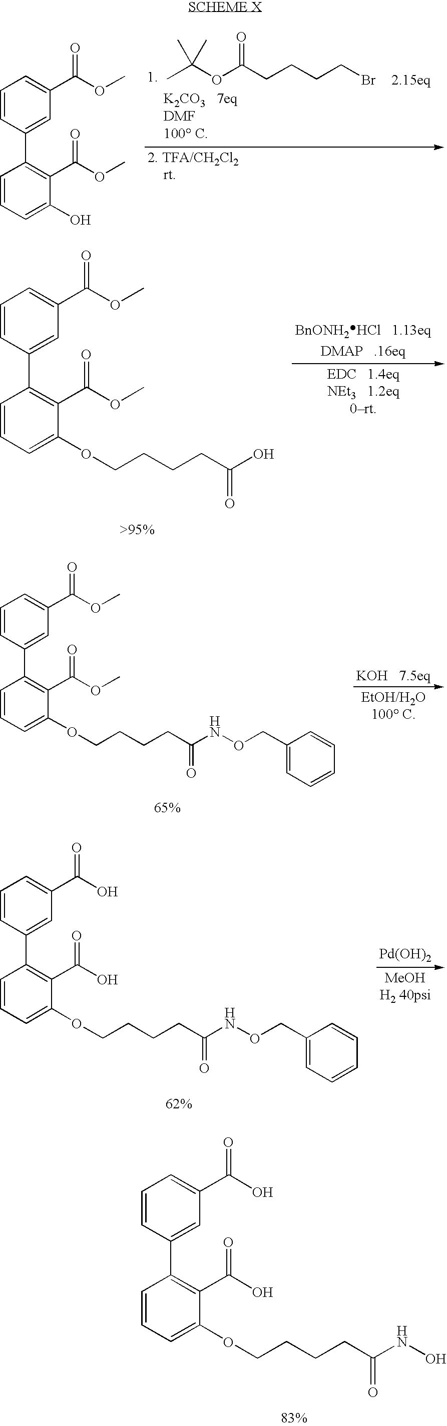

Precursor compounds may be commercially available, prepared by methods known to a person of skill in the art, or prepared by SCHEMES XI to XIV.

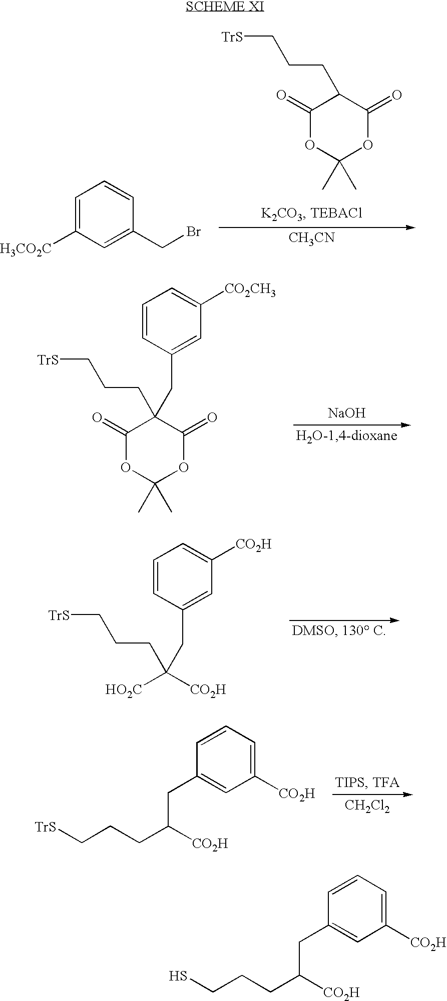

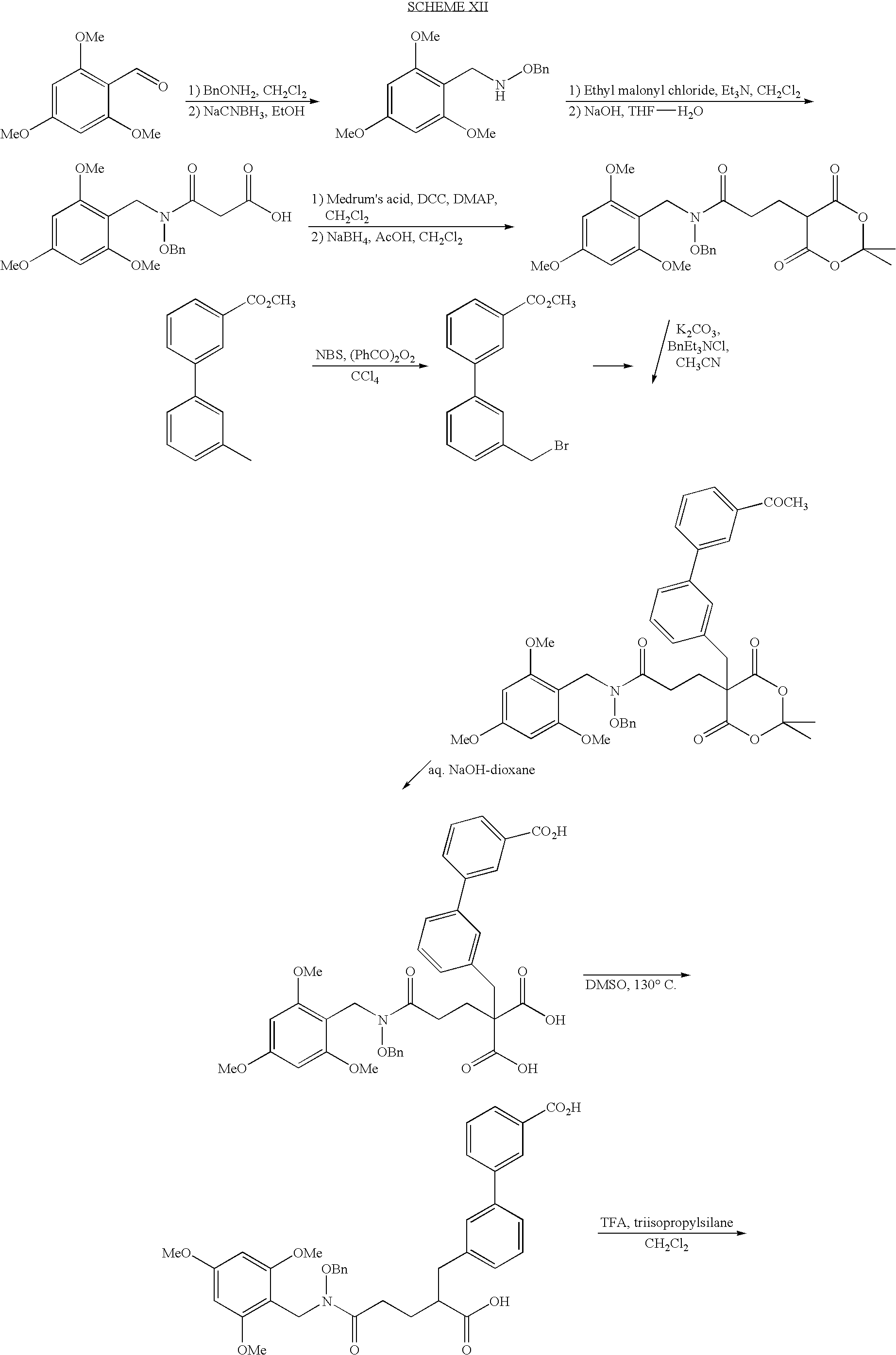

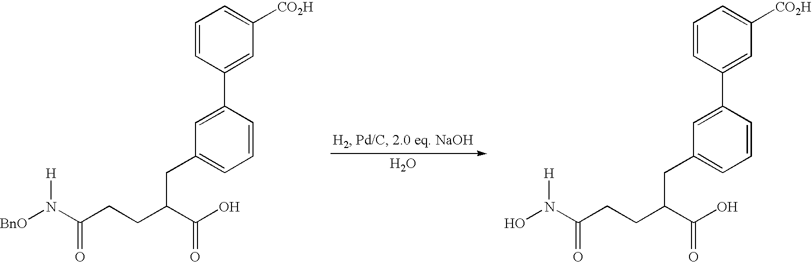

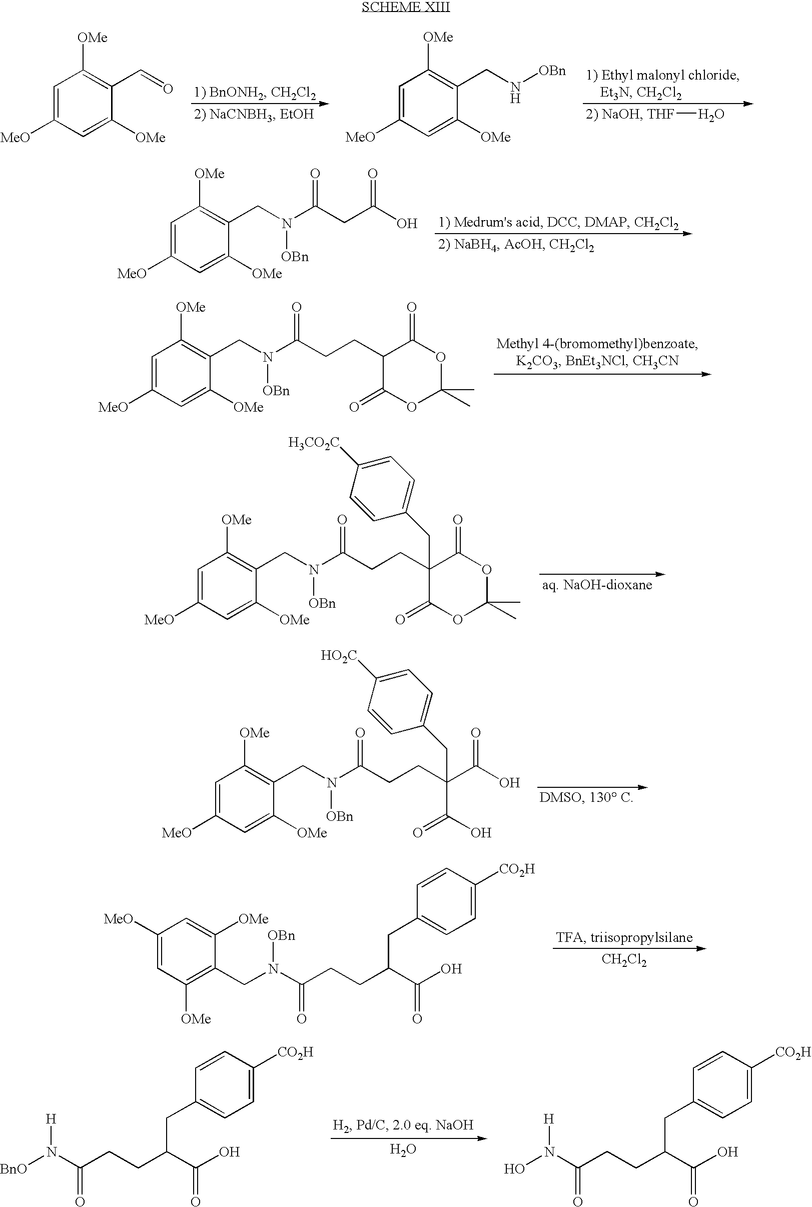

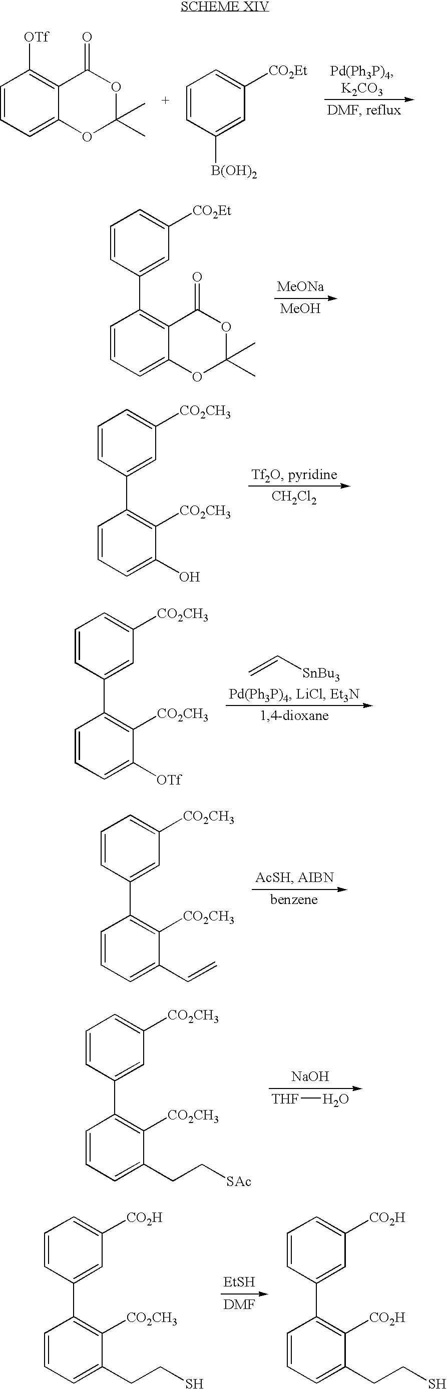

Route of Administration

In the inventive methods, the compounds will generally be administered to a patient in the form of a pharmaceutical formulation. Such formulation may include, in addition to the active agent, a physiologically acceptable carrier and/or diluent. The compounds may be administered locally or systemically by any means known to an ordinarily skilled artisan. For example, the compounds may be administered orally, parenterally, by inhalation spray, topically, rectally, nasally, buccally, vaginally or via an implanted reservoir in dosage formulations containing conventional non-toxic pharmaceutically acceptable carriers, adjuvants and vehicles. The term parenteral as used herein includes subcutaneous, intravenous, intraarterial, intramuscular, intraperitoneal, intrathecal, intraventricular, intrasternal, intracranial or intraosseous injection and infusion techniques. Topical administration includes, without limitation, administration via eyedrops. The exact administration protocol will vary depending upon various factors including the age, body weight, general health, sex and diet of the patient; the determination of specific administration procedures would be routine to an ordinarily skilled artisan. The compounds and compositions used in the inventive methods may be capable of crossing the blood-brain barrier.

Dosage

In the inventive methods, the compounds and compositions may be administered by a single dose, multiple discrete doses or continuous infusion. Pump means, particularly subcutaneous pump means, are useful for continuous infusion.

Dose levels on the order of about 0.001 to about 10,000 mg/kg of the active ingredient compound are useful in the inventive methods. In one embodiment, the dose level is about 0.1 mg/kg/d to about 1,000 mg/kg/d. In another embodiment, the dose level is about 1 mg/kg/d to about 100 mg/kg/d. The specific dose level for any particular patient will vary depending upon a variety of factors, including the activity and the possible toxicity of the specific compound employed; the age, body weight, general health, sex and diet of the patient; the time of administration; the rate of excretion; drug combination; the severity of the particular disease being treated; and the form of administration. Typically, in vitro dosage-effect results provide useful guidance on the proper doses for patient administration. Studies in animal models are also helpful. The considerations for determining the proper dose levels are well known in the art.

Administration Regimen

For the inventive methods, any administration regimen well known to an ordinarily skilled artisan for regulating the timing and sequence of drug delivery can be used and repeated as necessary to effect treatment. Such regimen may include pretreatment and/or co-administration with additional therapeutic agents.

Co-Administration with Other Treatments

In the inventive methods, the NAALADase inhibitors and pharmaceutical compositions may be used alone or in combination with one or more additional agent(s) for simultaneous, separate or sequential use.

The additional agent(s) may be any therapeutic agent(s) known to an ordinarily skilled artisan, including, without limitation, (an)other compound(s) of formulas I-XV.

The NAALADase inhibitors and pharmaceutical compositions may be co-administered with one or more therapeutic agent(s) either (i) together in a single formulation, or (ii) separately in individual formulations designed for optimal release rates of their respective active agent. Each formulation may contain from about 0.01% to about 99.99% by weight of a NAALADase inhibitor, as well as one or more pharmaceutically acceptable carrier(s), such as wetting, emulsifying and/or pH buffering agent(s).

In addition, the NAALADase inhibitors and pharmaceutical compositions may be administered prior to, during or following surgery or physical therapy.

### EXAMPLES

The following examples are illustrative of the present invention and are not intended to be limitations thereon. Unless otherwise indicated, all percentages are based upon 100% by weight of the final composition.

### Example 1

Preparation 3-(2-Mercaptoethyl)-[1,1′-biphenyl]-2,3′-dicarboxylic Acid

(Scheme I)

3-(2,2-Dimethyl-4-oxo-4H-1,3-benzodioxin-5-yl)-benzoic acid, ethyl ester

To a solution of 2,2-dimethyl-5-trifluoro-methanesulfonyloxy-4H-1,3-benzodioxin-4-one (2.0 g, 5.8 mmol), 3-ethoxycarbonylphenylboronic acid (1.34 g, 6.9 mmol) and anhydrous K₂CO₃ powder (2.61 g, 18.9 mmol) in DMF (30 mL) was added tetrakis(triphenylphosphine) palladium(0.202 g, 0.175 mmol). The mixture was heated at reflux for 2 hours. The reaction mixture was allowed to cool to room temperature and 1 N HCl (25 mL) was added. The mixture was extracted with EtOAc (3×25 mL). The combined extracts were washed with water and brine, then dried over MgSO₄, filtered, and concentrated. The crude material was purified by flash chromatography (1:15 EtOAc/hexanes) to afford 3-(2,2-dimethyl-4-oxo-4H-1,3-benzodioxin-5-yl)-benzoic acid, ethyl ester (1.2 g, 63%) as a white solid: ¹H NMR (CDCl₃) δ 1.39 (t, J=7.1 Hz, 3H), 1.80 (s, 6H), 4.39 (q, J=7.0 Hz, 2H), 7.01 (d, J=8.0 Hz, 2H), 7.47-7.57 (m, 3H), 8.00 (t, J=1.5 Hz, 1H), 8.07 (dt, J=7.5, 1.5 Hz, 1H).

3-Hydroxy-[1,1′-biphenyl]-2,3′-dicarboxylic acid, dimethyl ester

To a solution of 3-(2,2-dimethyl-4-oxo-4H-1,3-benzodioxin-5-yl)benzoic acid, ethyl ester (1.4 g, 4.3 mmol) in methanol (10 mL) was added sodium methoxide (0.5 M in methanol, 25 mL) at 0 ° C. The solution was stirred at room temperature for 15 minutes. The reaction was quenched by addition of 1 N HCl (30 mL) and extracted with EtOAc (3×30 mL). The combined organic extracts were dried over MgSO₄ and concentrated to afford 3-hydroxy-[1,1′-biphenyl]-2,3′-dicarboxylic acid, dimethyl ester (1.2 g, 95%) as a yellow solid: ¹H NMR (CDCl₃) δ 3.43 (s, 3H), 3.93 (s, 3H), 6.79 (dd, J=7.5, 0.9 Hz, 1H), 7.04 (dd, J=7.5, 0.9 Hz, 1H), 7.43 (m, 3H), 7.93 (m, 1H), 8.02 (dm, J=7.0 Hz, 1H), 10.8 (s, 1H).

3-Trifluoromethanesulfonyloxy-[1,1′-biphenyl]-2,3′-dicarboxylic acid, dimethyl ester

To a solution of 3-hydroxy-[1,1′-biphenyl]-2,3′-dicarboxylic acid, dimethyl ester (1.1 g, 3.8 mmol) in dichloromethane (15 mL) were added pyridine (1.00 mL, 12.3 mmol) and trifluromethanesulfonic anhydride (0.90 mL, 5.4 mmol) at 0° C. The solution was stirred at 0° C for 2 hours. Aqueous 1 N HCl (20 mL) was added, and the mixture was extracted with dichloromethane (3×20 mL). The combined organic extracts were washed with water and brine, dried over MgSO₄, filtered, and concentrated to give 3-trifluoromethanesulfonyloxy-[1,1′-biphenyl]-2,3′-dicarboxylic acid, dimethyl ester (1.4 g, 87%) as a yellow solid: ¹H NMR (CDCl₃) □3.72 (s, 3H), 3.94 (s, 3H), 7.38-7.62 (m, 5H), 8.08 (m, 2H).

3-Ethenyl-[1,1′-biphenyl]-2,3′-dicarboxylic acid, dimethyl ester

A mixture of 3-trifluoromethanesulfonyloxy-[1,1′-biphenyl]-2,3′-dicarboxylic acid, dimethyl ester (1.3 g, 3.1 mmol), tetrakis(triphenylphosphine)palladium (0.36 g, 0.31 mmol), LiCl (0.94 g, 22.2 mmol), triethylamine (0.6 mL, 4.3 mmol) and tri-n-butyl(vinyl)tin (1.0 mL, 3.4 mmol) in 1,4-dioxane (30 mL) was heated at reflux under N₂ for 4 hours. After cooling to room temperature, the mixture was filtered through a plug of silica gel and the filtrate was concentrated. Purification by flash chromatography (1:10 EtOAc/hexanes) provided 3-ethenyl-[1,1′-biphenyl]-2,3′-dicarboxylic acid, dimethyl ester (0.91 g, 99%) as a white solid: ¹H NMR (CDCl₃) δ 3.61 (s, 3H), 3.92 (s, 3H), 5.40 (d, J=11.1 Hz, 1H), 5.79 (d, J=17.5 Hz, 1H), 6.87 (dd, J=17.4, 11.0 Hz, 1H), 7.31 (d, J=7.5 Hz, 1H), 7.44-7.49 (m, 2H), 7.56 (dm, J=7.5 Hz, 1H), 7.62 (d, J=8.0 Hz, 1H), 8.03 (dm, J=7.5 Hz, 1H), 8.08 (t, 1,J=1.5 Hz, 1H).

3-[2-(Acetylthio)ethyl]-1,1′-biphenyl]-2,3′-dicarboxylic acid, dimethyl ester

To a solution of 3-ethenyl-[1,1′-biphenyl]-2,3′-dicarboxylic acid, dimethyl ester (0.85 g, 2.9 mmol) in benzene (10 mL) was added thioacetic acid (2.1 mL, 29.4 mmol) followed by AIBN (0.053 g, 0.32 mmol). The solution was deoxygenated for 30 minutes by bubbling nitrogen through the solution and then heated at reflux for 4 hours. Saturated aqueous NaHCO₃ (20 mL) was added to the solution and the mixture was extracted with EtOAc (2×20 mL). The combined organic extracts were washed with water and brine, dried over MgSO₄, filtered, and concentrated. The residue was purified by flash chromatography (1:12 EtOAc/hexanes) to give 3-[2-(acetylthio)ethyl]-[1,1′-biphenyl]-2,3′-dicarboxylic acid, dimethyl ester (0.51 g, 48%) as an off white solid: ¹H NMR (CDCl₃) δ 2.35 (s, 3H), 2.93 (m, 2H), 3.14 (m, 2H), 3.62 (s, 3H), 3.93 (s, 3H), 7.29 (dd,J=7.6, 0.9 Hz, 1H), 7.35 (dd, J=7.5, 0.8 Hz, 1H), 7.44 (d, J=7.6 Hz, 1H), 7.48 (d, J=7.5 Hz, 1H), 7.55 (dt, J=8.0, 1.5 Hz, 1H), 8.03 (dt, J=7.9, 1.5 Hz, 1H), 8.07 (t, J=1.5 Hz, 1H).

3-(2-Mercaptoethyl)-[1,1′-biphenyl]-2,3′-dicarboxylic acid, 2-methyl ester

To a deoxygenated solution of 3-[2-(acetylthio) ethyl]-[1,1′-biphenyl]-2,3′-dicarboxylic acid, dimethyl ester (0.50 g, 1.34 mmol) in THF (3.5 mL) was added a deoxygenated solution of NaOH (0.38 g, 9.4 mmol) in water (3.5 mL). The mixture was stirred overnight, and 1 N HCl (20 mL) was added. The mixture was extracted with EtOAc (3×20 mL). The combined organic extracts were washed with water and brine, dried over MgSO₄, filtered, and concentrated to afford 3-(2-mercaptoethyl)-[1,1′-biphenyl]-2,3′-dicarboxylic acid, 2-methyl ester (0.35 g, 83%) as an off white solid: ¹H NMR (CDCl₃) δ 1.46 (t, J=8.0 Hz, 1H), 2.83 (m, 2H), 3.00 (m, 2H), 3.60 (s, 3H), 7.33-7.31 (m, 2H), 7.46 (t, J=7.7 Hz, 1H), 7.52 (t, J=7.7 Hz, 1H), 7.61 (dm, J=7.9 Hz, 1H), 8.10 (dm, J=7.9 Hz, 1H), 8.14 (m, 1H).

3-(2-Mercaptoethyl)-[1,1′-biphenyl]-2,3′-dicarboxylic acid

To a deoxygenated suspension of sodium ethanethiolate (0.135 g, 1.60 mmol) in DMF (0.5 mL) was added a solution of 3-(2-mercaptoethyl)-[1,1′-biphenyl]-2,3′-dicarboxylic acid, 2-methyl ester (0.10 g, 0.32 mmol) in DMF (0.5 mL). Argon was bubbled through the mixture for 10 minutes. The reaction was heated at 100° C. for 1 hour and 200° C. for another hour. After the mixture cooled to room temperature, the reaction was quenched with 1 N HCl (20 mL) and was extracted with EtOAc (3×20 mL). The combined organic extracts were washed with water and brine, dried over MgSO₄, filtered, and concentrated to afford 3-(2-mercaptoethyl)-[1,1′-biphenyl]-2,3′-dicarboxylic acid (0.055 g, 57%) as a white solid: ¹H NMR (CDCl₃) δ 1.51 (t, J=8.0 Hz, 1H), 2.87-2.93 (m, 2H), 3.12-3.08 (m, 2H), 7.37 (m, 2H), 7.57-7.47 (m, 2H), 7.70 (dm, J=7.9 Hz, 1H), 7.98 (dm, J=7.8 Hz, 1H), 8.30 (m, 1H); ¹³C NMR (CDCl₃)□26.2, 38.8, 128.1, 129.3, 129.7, 129.8, 129.9 (2C), 130.5, 133.3, 134.4, 137.7, 139.1, 141.2, 172.3, 176.9. Elemental analysis calculated for C₁₆H₁₄O₄S: C, 63.56; H, 4.67; S, 10.61. Found: C, 63.65; H, 4.88; S, 10.33.

### Example 2

Preparation of 2-[(4-carboxyphenyl)methoxy]-6-(2-mercaptoethyl)-benzoic Acid

(Scheme II)

5-Ethenyl-2,2-dimethyl-4H-1,3-benzodioxin-4-one

A mixture of 2,2-dimethyl-5-trifluoromethanesulfonyloxy-4H-1,3-benzodioxin-4-one (9.90 g, 30.3 mmol), tributyl(vinyl)tin (10.10 g, 31.9 mmol), lithium chloride (8.70 g, 205 mmol), and triethylamine (5.0 mL, 36.0 mmol) in 1,4-dioxane (300 mL) was deoxygenated by bubbling nitrogen through the mixture for 1 hour. To the mixture was added tetrakis(triphenylphosphine)palladium (3.40 g, 2.90 mmol) and the mixture was heated at 100° C. for 3 hours. The mixture was allowed to cool to room temperature and was filtered. The filtrate was concentrated and purified by flash chromatography (1:12, EtOAc/hexanes) to provide 5-ethenyl-2,2-dimethyl-4H-1,3-benzodioxin-4-one (5.00 g, 81%) as a yellow oil: ¹H NMR: (CDCl₃) δ 1.72 (s, 6H), 5.43 (dd, J=11.0, 1.3 Hz, 1H), 5.72 (dd, J=17.5, 1.3 Hz, 1H), 6.89 (d, J=8.0 Hz, 1H), 7.27 (d, J=8.0 Hz, 1H), 7.47 (t, J=8.0 Hz, 1H), 7.73 (dd, J=17.5, 11.0 Hz, 1H).

2-Ethenyl-6-hydroxybenzoic acid, methyl ester

To 5-ethenyl-2,2-dimethyl-4H-1,3-benzodioxin-4-one (4.01 g, 19.6 mmol) was added 0.5 M sodium methoxide in methanol (85 mL, 42.5 mmol) at room temperature. Aqueous 1 N HCl (100 mL) was added to the solution after 15 minutes. The cloudy solution was extracted with ether (2×100 mL). The combined organic extracts were washed with H₂O (50 mL) and brine (50 mL), dried over Na₂SO₄, filtered, and concentrated to afford 2-ethenyl-6-hydroxybenzoic acid, methyl ester (2.0 g, 57%) as a yellow oil. This material was used without further purification in the next step: ¹H NMR (CDCl₃) δ 3.96 (s, 3H), 5.26 (dd, J=10.8, 1.5 Hz, 1H), 5.49 (dd, J=17.3, 1.5 Hz, 1H), 6.95 (m, 2H), 7.23-7.39 (m, 2H), 11.12(s, 1H).

2-Ethenyl-6-[4-(methoxycarbonyl)phenyl]methoxy-benzoic acid, methyl ester

To a stirred solution of the above material (0.500 g, 2.8 mmol) in acetone (10 mL) were added K₂CO₃ (1.50 g, 10.9 mmol) and methyl 4-(bromomethyl)benzoate (0.71 g, 3.10 mmol) at room temperature. The mixture was stirred under nitrogen for 3 hours and filtered. The filtrate was concentrated and residue was purified by flash chromatography (1:10 EtOAc/hexanes) to provide 2-ethenyl-6-[4-(methoxycarbonyl)phenyl]methoxy-benzoic acid, methyl ester (0.73 g, 80%) as a white solid: ¹H NMR (CDCl₃) 6 3.92 (s, 6H), 5.17 (s, 2H), 5.37 (dd, J=11.1, 1.0 Hz, 1H), 5.78 (dd, J=17.6, 0.9 Hz, 1H), 6.70 (dd, 1, J=17.4, 11.1 Hz, 1H), 6.83 (d, J=7.8 Hz, 1H), 7.20 (d, J=8.0 Hz, 1H) 7.29 (t, J=8.0 Hz, 1H), 7.46 (d, J=8.4 Hz, 2H), 8.04 (d, J=8.3 Hz, 2H).

2-[2-(Acetylthio)ethyl]-6-[4-(methoxycarbonyl)phenyl]-methoxy-benzoic acid, methyl ester

To a solution of 2-ethenyl-6-[4-(methoxycarbonyl)phenyl]methoxy-benzoic acid, methyl ester (0.71 g, 2.18 mmol) in benzene (10 mL) was added thioacetic acid (1.80 mL, 25.2 mmol) followed by AIBN (37 mg, 0.23 mmol). After nitrogen was bubbled through the solution for 30 minutes, the solution was heated at reflux for 4 hours. The reaction was allowed to cool to room temperature and saturated NaHCO₃ (20 mL) was added. The mixture was extracted with EtOAc (3×20 mL). The combined organic extracts were washed with water and brine, dried over MgSO₄, filtered and concentrated in vacuo. The residue was purified by flash chromatography (1:10 EtOAc/hexanes) to give 2-[2-(acetylthio)ethyl]-6-[4-(methoxycarbonyl)-phenyl]methoxy-benzoic acid, methyl ester (0.50 g, 60%) as a clear oil: ¹H NMR (CDCl₃) δ 2.34 (s, 3H), 2.85-2.82 (m, 2H), 3.07-3.10 (m, 2H), 3.92 (s, 3H), 3.94 (s, 3H), 5.16 (s, 2H), 6.81 (d, J=8.2 Hz, 1H), 6.92 (d, J=7.7 Hz, 1H), 7.28 (t, J=8.2 Hz, 1H), 7.46 (d, J=8.3 Hz, 2H), 8.04 (d, J=8.3 Hz, 2H).

2-[(4-Carboxyphenyl)methoxy]-6-(2-mercaptoethyl)-benzoic acid

To a deoxygenated solution of 2-[2-(acetylthio)ethyl]-6-[4-(methoxycarbonyl)phenyl]methoxy-benzoic acid, methyl ester (0.20 g, 0.50 mmol) in 95% EtOH (3 mL) was added a deoxygenated solution of KOH (0.463 g, 8.3 mmol) in 95% EtOH (3 mL) under nitrogen. The solution was heated at reflux overnight and quenched by addition of 1 N HCl (20 mL). The mixture was extracted with EtOAc (3×20 mL) and the combined organic extracts were washed with water and brine, then dried over MgSO₄, filtered, and concentrated. Purification by flash chromatography (1:1 dichloromathane/hexanes with 1% acetic acid) provided 2-[(4-carboxyphenyl)methoxy]-6-(2-mercaptoethyl)-benzoic acid (0.077 g, 46%) as a white solid: ¹H NMR (CD₃OD) δ 2.75 (m, 2H), 2.92 (m, 2H), 5.22 (s, 2H), 6.93 (d, J=7.5 Hz, 1H), 6.98 (d, J=8.2 Hz, 1H), 7.30 (t, J=8.3 Hz, 1H), 7.55 (d, J=7.9 Hz, 2H), 8.02 (d, J=8.0 Hz, 2H); ¹³C NMR (CD₃OD) □26.5, 39.8, 71.1, 112.4, 123.9, 126.9, 128.3, 131.3, 131.7, 131.8, 139.9, 144.2, 156.7, 170.0, 172.3. Elemental analysis calculated for C₁₇H₁₆O₅S: C, 61.43; H, 4.85; S, 9.65. Found: C, 61.16; H, 4.95; S, 9.44.

### Example 3

Preparation of 4-mercaptomethyl-[1,1′-biphenyl]-2,3′-dicarboxylic acid

(Scheme III)

4-Methyl-[1,1′-biphenyl]-2,3′-dicarboxylic acid

To a solution of 2-bromo-5-methylbenzoic acid (5.00 g, 23.3 mmol) in DME (100 mL) were added 3-carboxyphenylboronic acid (3.86 g; 23.3 mmol), a solution of Na₂CO₃ (9.90 g, 93 mmol) in H₂O and tetrakis(triphenylphosphine)palladium. The mixture was stirred at 90° C. for 4 days. The mixture was allowed to cool to room temperature, diluted with EtOAc (50 mL), and washed with a saturated NaHCO₃ solution. The aqueous layer was separated, acidified with 10% HCl, and extracted with EtOAc (3×20 mL). The combined extracts were dried over MgSO₄ and concentrated. The crude material was purified by column chromatography (9:1 hexanes/EtOAc 1% acetic acid) to afford 4-methyl-[1,1′-biphenyl]-2,3′-dicarboxylic acid (2.20 g, 37%) as a solid: ¹H NMR (DMSO-d₆) δ 2.40 (s, 3H), 7.30 (m, 1H), 7.42 (m, 1H), 7.52-7.57 (m, 2H), 7.60 (s, 1H), 7.85 (s, 1H), 7.91-7.92 (m, 1H).

4-Methyl-[1,1′-biphenyl]-2,3′-dicarboxylic acid, dimethyl ester

To a solution of 4-methyl-[1,1′-biphenyl]-2,3′-dicarboxylic acid (2.20 g, 8.6 mmol) in methanol (150 mL) was added conc. H₂SO₄ (1.6 mL) and the mixture was heated at reflux overnight. The solvent was removed under a reduced pressure and the residue was partitioned between saturated aqueous NaHCO₃ solution and EtOAc (20 mL). The organic layer was dried over MgSO₄ and concentrated to give 4-methyl-[1,1′-biphenyl]-2,3′-dicarboxylic acid, dimethyl ester (2.26 g, 92%) as a crude material. This product was used for the next reaction without further purification: ¹H NMR (DMSO-d₆) δ 2.41 (s, 3H), 3.58 (s, 3H), 3.88 (s, 3H), 7.36-7.38 (m, 1H), 7.47-7.48 (m, 1H), 7.56-7.58 (m, 2H), 7.62 (s, 1H), 7.82 (s, 1H), 7.94-7.96 (m, 1H).

4-Bromomethyl-[1,1′-biphenyl]-2,3′-dicarboxylic acid, dimethyl ester

To a solution of 4-methyl-[1,1′-biphenyl]-2,3′-dicarboxylic acid, dimethyl ester (2.26 g, 7.9 mmol) in CCl₄ (50 mL) were added benzoyl peroxide (0.010 g, 0.04 mmol) and NBS (1.42 g, 8.0 mmol), and the mixture was refluxed for 3 days. The reaction mixture was allowed to cool to room temperature, filtered, and concentrated. The residue was purified by column chromatography (95:5 to 90:10 hexanes/EtOAc) to afford 4-bromomethyl-[1,1′-biphenyl]-2,3′-dicarboxylic acid, dimethyl ester (1.71 g, 60%): ¹H NMR (DMSO-d₆) δ 3.61(s, 3H), 3.88 (s, 3H), 4.84 (s, 2H), 7.48-7.50 (d, J=8.0 Hz, 1H), 7.59-7.60 (m, 2H), 7.72-7.75 (m, 1H), 7.85 (s, 1H), 7.90 (m, 1H), 7.97-7.99 (m, 1H).

4-Acetylthiomethyl-[1,1′-biphenyl]-2,3′-dicarboxylic acid, dimethyl ester

To a solution of 4-bromomethyl-[1,1′-biphenyl]-2,3′-dicarboxylic acid, dimethyl ester (1.59 g, 4.4 mmol) in acetone (75 mL) was added potassium thioacetate (0.60 g, 5.3 mmol), and the mixture was refluxed for 1 hour. The mixture was allowed to cool to room temperature, filtered, and concentrated. The residual product was purified by column chromatography (hexanes/EtOAc, 9/1) to afford 4-acetylthiomethyl-[1,1′-biphenyl]-2,3′-dicarboxylic acid, dimethyl ester (1.21 g, 76%): ¹H NMR (DMSO-d₆) δ 2.39 (s, 3H), 3.60 (s, 3H), 3.88 (s, 3H), 4.23 (s, 2H), 7.42-7.44 (d, J=8.0 Hz, 1H), 7.57-7.60 (m, 3H), 7.74 (s 1H), 7.83 (s, 1H), 7.96-7.99 (m, 1H).

4-Mercaptomethyl-[1.1 ′-biphenyl]-2,3′-dicarboxylic acid

To a solution of 4-acetylthiomethyl-[1,1′-biphenyl]-2,3′-dicarboxylic acid, dimethyl ester (0.27 g, 0.75 mmol) in deoxygenated THF was added a degassed solution of sodium hydroxide (0.12 g, 3.0 mmol) in H₂O (5 mL) at room temperature. After 24 hours, additional solution of sodium hydroxide (0.09 g) in H₂O (2 mL) was added to the reaction mixture and the mixture was stirred for 24 hours. The mixture was acidified with 10% HCl and extracted with EtOAc. The extract was dried over MgSO₄ and concentrated. The crude material was purified by column chromatography with (9:1 dichloromethane/EtOAc with 1% acetic acid) to afford 4-mercaptomethyl-[1,1′-biphenyl]-2,3′-dicarboxylic acid (0.20 g, 92%) as a white solid: ¹H NMR (DMSO-d) δ 3.10 (t, J=8.03 Hz, 1H), 3.89 (d, J=8.03 Hz, 2H), 7.43 (d, J=7.53 Hz, 1H), 7.58-7.65 (m, 3H), 7.83 (d, J=2.01 Hz, 1H), 7.92 (s, 1H), 7.97-8.00 (m, 1H). Elemental analysis calculated for C₁₅H₁₂O₄S.0.5 AcOH: C, 60.37; H, 4.43; O, 25.13; S, 10.07. Found: C, 60.28; H, 4.45; S, 10.15.

### Example 4

Preparation of 2-Carboxymethyl-6-(2-Mercaptoethyl)-Benzoic Acid)

(Scheme IV)

2-Hydroxy-6-methoxycarbonylmethyl-benzoic acid methyl ester

To a solution of 2-carboxymethyl-6-hydroxy-benzoic acid (5.021 g, 25.6 mmol) in DMF (100 mL) at 0° C. were added K₂CO₃ (3.567 g, 25.9 mmol) and CH₃I (7.9 mL, 51.9 mmol). After stirring under nitrogen at 0° C. for 4 h, the reaction was partitioned between H₂O (100 mL) and ether (150 mL). The aqueous layer was acidified with 1 N HCl and extracted with EtOAc (200 mL). The EtOAc layer was dried over MgSO₄ and concentrated in vacuo to give the crude 2-hydroxy-6-methoxycarbonylmethyl-benzoic acid methyl ester as a colorless oil (3.2 g, 56%): ¹H NMR (CDCl₃) δ 3.69 (s, 3H), 3.89 (s, SH), 6.72 (d, J=6.7 Hz, 1H), 6.96 (d, J=8.0 Hz, 1H), 7.33-7.38 (m, 1H).

2-Methoxycarbonylmethyl-6-trifluoromethanesulfonyloxy-benzoic acid methyl ester

To a solution of 2-hydroxy-6-methoxycarbonylmethyl-benzoic acid methyl ester (2.51 g, 11.2 mmol) in CH₂Cl₂ (50 mL) at 0° C. were added triflic anhydride (3.0 mL, 17.8 mmol) and pyridine (2.40 mL, 29.7 mmol). The solution was allowed to warm to rt overnight and was concentrated in vacuo. The residue was diluted with EtOAc (100 mL), washed with 1 N HCl (25 mL), saturated aqueous NaHCO₃ (25 mL), H₂O (25 mL), and brine (25 mL). The organic layer was dried over MgSO₄ and concentrated. The residue was purified by flash chromatography (10% EtOAc/hexanes) to provide 2-methoxycarbonylmethyl-6-trifluoromethanesulfonyloxy-benzoic acid methyl ester as an oil (3.5 g, 90%): ¹H NMR (CDCl₃) 6 3.86 (s, 3H), 4.06 (s, 2H), 4.10 (s, 3H), 7.4 (d, J=7.6 Hz, 1H), 7.50 (d, J=7.6 Hz, 1H), 7.67 (t, J=8.2 Hz, 1H).

2-Methoxycarbonylmethyl-6-vinyl-benzoic acid methyl ester

A mixture of 2-methoxycarbonylmethyl-6-trifluoromethanesulfonyloxy-benzoic acid methyl ester (2.705 g, 7.6 mmol), LiCl (2.355 g, 55.6 mmol), NEt₃ (1.5 mL, 10.8 mmol), Pd[P(C₆H₅)₃]₄ (1.09 g, 0.94 mmol), and tributyl (vinyl)tin (2.85 mL, 9.75 mmol) in dioxane (50 mL) was heated at reflux. After 3 h, the reaction was allowed to cool to rt, filtered through a pad of silica gel and concentrated. The crude product was purified by flash chromatography on SiO₂ (13% EtOAc/hexanes) to afford 2-methoxycarbonylmethyl-6-vinyl-benzoic acid methyl ester as a yellow solid (1.50 g, 84%): ¹H NMR (CDCl₃) δ 3.67 (s, 3H), 3.89 (s, 5H), 5.32 (d, J=10.9 Hz, 1H), 5.68 (d, J=17.4 Hz, 1H), 6.82 (dd, J=17.4, 10.9 Hz, 1H), 7.20 (d, J=7.1 Hz, 1H), 7.36 (m, 1H), 7.50 (d, J=7.8 Hz, 1H).

2-(2-Acetylsulfanyl-ethyl)-6-methoxycarbonylmethyl-benzoic acid methyl ester

Nitrogen was bubbled through a solution of 2-methoxycarbonylmethyl-6-vinyl-benzoic acid methyl ester (1.31 g, 5.60 mmol), thioacetic acid (4.00 mL, 56.0 mmol), and AIBN (0.184 g, 1.10 mmol) in benzene (30 mL) for 3 h at rt to remove oxygen. The mixture was heated at reflux for 3 h. The solution was allowed to cool to rt and saturated aqueous NaHCO₃ (100 mL) was added. The mixture was extracted with EtOAc (100 mL) and the organic layer was washed with H₂O (100 mL) and brine (100 mL), then dried over MgSO₄ and concentrated in vacuo. Flash chromatography on SiO₂ (10% EtOAc/hexanes) provided 2-(2-acetylsulfanyl-ethyl)-6-methoxycarbonylmethyl-benzoic acid methyl ester as a yellow solid (0.555 g, 33%): ¹H NMR (CDCl₃): δ 2.40 (s, 3H), 2.94-2.99 (m, 2H), 3.13-3.18 (m, 2H), 3.75 (s, 3H), 3.78 (s, 2H), 3.99 (s, 3H), 7.25 (d, J=7.6 Hz, 1H), 7.32 (d, J=7.6 Hz, 1H), 7.42 (t, J=7.6 Hz, 1H).

2-Carboxymethyl-6-(2-mercapto-ethyl)-benzoic acid (48)

A deoxygenated mixture of 2-(2-acetylsulfanyl-ethyl)-6-methoxycarbonylmethyl-benzoic acid methyl ester (0.555 g, 1.77 mmol) and 6 N KOH (3 mL) in EtOH (7 mL) was heated at reflux for 18 h. After allowing to cool to rt, the mixture was partitioned between 1 N HCl (75 mL) and EtOAc (100 mL). The organic layer was washed with H₂O (100 mL), brine (100 mL), dried over MgSO₄ and concentrated in vacuo. The crude product was dissolved in ether (100 mL), filtered to remove some unidentified solid material, and concentrated to give 2-carboxymethyl-6-(2-mercapto-ethyl)-benzoic acid as a white solid (0.310 g, 73%): ¹H NMR (CDCl₃) δ 1.37 (t, J=8.2 Hz, 1H), 2.76 (m, 2H), 3.06 (m, 2H), 3.90 (s, 2H), 7.10 (d, J=7.6 Hz, 1H), 7.21 (d, J=6.9 Hz, 1H), 7.34 (t, J=7.8 Hz, 1H): ¹³C NMR (CDCl₃) □ 27.25, 40.43, 41.83, 131.58, 131.64, 132.45, 132.55, 134.66, 141.41, 176.14, 179.40. Elemental analysis calculated for C₁₁H₁₂O₄S: C, 54.99; H, 5.03; S, 13.35. Found: C, 55.20; H, 5.26; S, 13.32.

### Example 5

Preparation of Precursor Compound 4-[2-Carboxy-5-(Hydroxyamino)-5-Oxopentyl]Benzoic Acid

(Scheme XIII)

O-Benzyl-N-(2,4,6-trimethoxy-benzyl)hydroxylamine

2,4,6-Trimethoxybenzaldehyde (12.42 g, 63.3 mmol) and 0-benzylhydroxylamine (7.80 g, 63.3 mmol) were stirred in dichloromethane (65 mL) at rt overnight. The solvent was removed under reduced pressure. The crude residue was dissolved in ethanol (550 mL) and sodium cyanoborohydride (12.0 g, 189.9 mmol) was added dropwise. The mixture was stirred for 5-10 min, then 12 N HCl was added dropwise until pH became below 3. The reaction mixture was stirred for additional 3 h and 1 N NaOH was added until pH>8. The product was extracted with dichloromethane. The organic phase was dried over Na₂SO₄ and concentrated under vacuo. The residue was purified by flash chromatography (5% ether in dichloromethane) to give 15.9 g of O-benzyl-N-(2,4,6-trimethoxy-benzyl)hydroxylamine as an oil (83% yield).

3-[Benzyloxy(2,4,6-trimethoxybenzyl)amino]-3-oxopropanoic acid

To a solution of O-benzyl-N-(2,4,6-trimethoxy-benzyl)hydroxylamine (2.0 g, 6.59 mmol) in dichloromethane (15 mL) was added triethylamine (1.0 mL, 7.25 mmol). The solution was cooled at 0° C. and ethyl malonyl chloride (0.85 mL, 6.59 mmol) was added. Precipitation was gradually formed. The resulting yellow slushy mixture was stirred at 0° C. for 15 min then brought up to rt and stirred for overnight. The solvent was removed under reduced pressure and the crude product was dissolved in EtOAc. The organic solution was consecutively washed with aq. 10% KHSO₄ and aq. saturated NaHCO₃. The organic layer was dried over Na₂SO₄ and concentrated. The crude material was stirred in H₂O-THF (1:1 by volume, 30 mL) containing NaOH (1.32 g, 33 mmol) for 1 h. The reaction mixture was washed with EtOAc and the aqueous layer was acidified with aq. 10% KHSO₄ solution. The product was extracted with EtOAc, dried over Na₂SO₄, and concentrated to give 1.62 g of 3-[benzyloxy(2,4,6-trimethoxybenzyl)amino]-3-oxopropanoic acid as a white solid (63% yield).

N-Benzyloxy-3-(2,2-dimethyl-4,6-dioxo-1 3-dioxan-5-yl)-N-(2,4,6-trimethoxybenzyl)-propanamide

To a solution of 3-[benzyloxy(2,4,6-trimethoxybenzyl)amino]-3-oxopropanoic acid (9.41 g, 24.16 mmol), Meldrum's acid (3.84 g, 26.58 mmol), and DMAP (3.54 g, 29.0 mmol) in dichloromethane (125 mL) was dropwise added a solution of DCC (5.5 g, 26.6 mmol) in dichloromethane (25 mL) via addition funnel over a period of 1 h at 0° C. The mixture was left in the refrigerator overnight. DCU was filtered off and the filtrate was washed with 5% KHSO₄ (3 times) and brine and dried over MgSO₄ for 4 h in the refrigerator. The drying agent was removed by filtration and acetic acid (16 mL) was added to the filtrate. The mixture was again cooled at 0° C. and sodium borohydride (2.3 g, 60.4 mmol) was added in small portions while stirring over 1 h. The reaction mixture was left overnight in the refrigerator. The next day, the solution was washed 3 times with brine and 2 times with water. The organic layer was concentrated in vacuo and the crude material was recrystallized from EtOAc-hexanes mixture to give 8.08 g of N-benzyloxy-3-(2,2-dimethyl-4,6-dioxo-1,3-dioxan-5-yl)-N-(2,4,6-trimethoxybenzyl)-propanamide as a white solid (67% yield).

5-{3-[Benzyloxy(2,4,6-trimethoxybenzyl)amino]-3-oxopropyl}-2,2-dimethyl-5-[4-(methoxycarbonyl)phenyl]methyl-[1,3]dioxane-4,6-dione

To stirring acetonitrile were added N-benzyloxy-3-(2,2-dimethyl-4,6-dioxo-1,3-dioxan-5-yl)-N-(2,4,6-trimethoxybenzyl)-propanamide (10.0 g, 19.9 mmol), methyl 4-(bromomethyl)benzoate (5.02 g, 21.9 mmol), potassium carbonate (4.125 g, 29.9 mmol), and benzyltriethylammonium chloride (6.799 g, 29.9 mmol). This reaction mixture was heated to 65° C. for 5 h. The reaction mixture was then allowed to cool and extracted with 100 mL of 10% KHSO₄, 50 mL (3 times) of EtOAc, brine and then dried over Na₂SO₄. The crude product was purified by flash column chromatography (40% EtOAc in hexanes) to give 10.01 g of 5-{3-[benzyloxy(2,4,6-trimethoxybenzyl)amino]-3-oxopropyl}-2,2-dimethyl-5-[4-(methoxycarbonyl)phenyl]methyl-[1,3]dioxane-4,6-dione as a white powder (78% yield).

2-{3-[Benzyloxy(2,4,6-trimethoxybenzyl)amino]-3-oxopropyl}-2-(4-carboxyphenyl)methyl-malonic acid

To 5-{3-[benzyloxy(2,4,6-trimethoxybenzyl)amino]-3-oxopropyl}-2,2-dimethyl-5-[4-(methoxycarbonyl)phenyl]methyl-[1,3]dioxane-4,6-dione (5.38 g, 8.3 mmol) were added 50 mL of water, then 40 mL of 1,4-dioxane and finally a solution of NaOH (1.65 g, 41.4 mmol) in 20 mL of water. This mixture was heated at 100° C. for 2h. The solution was then allowed to cool to rt. The solvent was removed under reduced pressure and the residue was partitioned between 100 mL of 10% KHSO₄ and 100 mL of EtOAc. The organic layer was washed with brine, dried over Na₂SO₄, and concentrated to give 4.5 g of 2-{3-[benzyloxy(2,4,6-trimethoxybenzyl)amino]-3-oxopropyl}-2-(4-carboxyphenyl)-methylmalonic acid as an off white powder (95% yield).

4-[5-[Benzyloxy(2,4,6-trimethoxybenzyl)amino]-2-carboxy-5-oxopentyl]benzoic acid

2-{3-[Benzyloxy(2,4,6-trimethoxybenzyl)amino]-3-oxopropyl} -2-(4-carboxyphenyl)methyl-malonic acid (9.30 g, 15.6 mmol ) was taken up in DMSO (10 mL). This solution was heated at 130° C. for 1.5 h. The reaction mixture was then allowed to come to room temperature. The solvent was then removed under reduced pressure. EtOAc was used as an azeotrope to remove the DMSO. The residue was partitioned between 50 mL of 10% KHSO₄ and 100 mL of EtOAc. The organic layer was washed with brine (50 mL), dried over Na₂SO₄, and concentrated to give 6.17 g of 4-[5-[benzyloxy(2,4,6-trimethoxybenzyl)amino]-2-carboxy-5-oxopentyl]benzoic acid as an off white powder (76% yield). ¹H NMR (CDCl3) d 0.88-0.96 (m, 2H), 1.26-1.91 (m, 2H), 2.02-2.32 (m,2H), 2.47-2.83 (m, 2H), 3.01-3.60 (m,2H), 4.70 (s,1H), 7.14-7.29 (m,9H), 7.93-7.96 (d, 2H).

4-[5-(Benzyloxyamino)-2-carboxy-5-oxopentyl]benzoic acid

To stirring dichloromethane were added 4-[5-(benzyloxyamino)-2-carboxy-5-oxopentyl]benzoic acid (3.00 g, 5.4 mmol), then triisopropylsilane (0.855 g, 5.4 mmol) and TFA (4.7 mL).This mixture was stirred for 1.15 h. The solvent was then removed under reduced pressure. Dichloromethane (100 mL×5) was used to azeotrope excess TFA. The residual solids were triturated with 1:1 EtOAc/hexanes containing 1.0% AcOH to give 1.62 g of 4-[5-(benzyloxyamino)-2-carboxy-5-oxopentyl]benzoic acid as an off white colored powder (81% yield). ¹H NMR (MeOH-d₆) δ 0.20-0.35 (m, 2H), 0.43-0.63 (m, 2H), 1.08-1.14 (m, 1H), 1.27-1.46 (m, 2H), 3.26 (s, 2H), 5.73-5.84 (m, 7H), 6.36-6.39 (d, 2H)

4-[2-Carboxy-5-(hydroxyamino)-5-oxotpentyl]benzoic acid disodium salt

A 100-mL three neck round bottomed flask was charged with the 4-[5-(benzyloxyamino)-2-carboxy-5-oxopentyl]benzoic acid (0.500 g, 1.3 mmol), 0.5 N NaOH (5.2 mL, 2.6 mmol), water (8.3 ml), and a spatula full of 10% Pd on carbon. The mixture was stirred under hydrogen (1 atm) for 7 h. The catalyst was removed by filtration and the filtrate was lyophilized to give 0.382 g of 4-[2-carboxy-5-(hydroxyamino)-5-oxopentyl]benzoic acid disodium salt as an off white powder (89% yield): ¹H NMR (D₂O) δ 1.75-1.90 (m, 2H), 2.10-2.30 (m, 2H), 2.45-2.60 (m, 1H), 2.75-3.00 (m, 2H), 7.34 (d, J=8.0 Hz, 2H), 7.82 (d, J=8.0 Hz, 2H). Elemental analysis calculated for C₁₃H₁₃NNa₂O₆.0.78H₂O: C, 46.02; H, 4.33; N, 4.13. Found: C,46.06; H, 4.14; N, 3.79.

### Example 6

Preparation of Precursor Compound 3-(2-Mercaptoethyl)-[1,1′-biphenyl]-2,3′-dicarboxylic Acid (Scheme XI)

3-(2,2-Dimethyl-4-oxo-4H-1,3-benzodioxin-5-yl)-benzoic acid, ethyl ester

To a solution of 2,2-dimethyl-5-trifluoromethanesulfonyloxy-4H-1,3-benzodioxin-4-one (2.0 g, 5.8 mmol), 3-ethoxycarbonylphenylboronic acid (1.34 g, 6.9 mmol) and anhydrous K2CO3 powder (2.61 g, 18.9 mmol) in DMF (30 mL) was added tetrakis(triphenylphosphine)palladium (0.202 g, 0.175 mmol). The mixture was heated at reflux for 2 h. The reaction mixture was allowed to cool to room temperature (“rt”) and 1 N HCl (25 mL) was added. The mixture was extracted with EtOAc (3×25 mL). The combined extracts were washed with water and brine, then dried over MgSO₄, filtered, and concentrated. The crude material was purified by flash chromatography (1:15 EtOAc/hexanes) to afford 3-(2,2-dimethyl-4-oxo-4H-1,3-benzodioxin-5-yl)-benzoic acid, ethyl ester (1.2 g, 63%) as a white solid: ¹H NMR (CDCl₃) δ 1.39 (t, J=7.1 Hz, 3H), 1.80 (s, 6H), 4.39 (q, J=7.0 Hz, 2H), 7.01 (d, J=8.0 Hz, 2H), 7.47-7.57 (m, 3H), 8.00 (t, J=1.5 Hz, 1H), 8.07 (dt, J=7.5, 1.5 Hz, 1H).

3-Hydroxy-[1,1′-biphenyl]-2,3′-dicarboxylic acid dimethyl ester

To a solution of 3-(2,2-dimethyl-4-oxo-4H-1,3-benzodioxin-5-yl)benzoic acid, ethyl ester (1.4 g, 4.3 mmol) in methanol (10 mL) was added sodium methoxide (0.5 M in methanol, 25 mL) at 0° C. The solution was stirred at rt for 15 min. The reaction was quenched by addition of 1 N HCl (30 mL) and extracted with EtOAc (3×30 mL). The combined organic extracts were dried over MgSO4 and concentrated to afford 3-hydroxy-[1,1′-biphenyl]-2,3′-dicarboxylic acid, dimethyl ester (1.2 g, 95%) as a yellow solid: ¹H NMR (CDCl₃) δ 3.43 (s, 3H), 3.93 (s, 3H), 6.79 (dd, J=7.5, 0.9 Hz, 1H), 7.04 (dd, J=7.5, 0.9 Hz, 1H), 7.43 (m, 3H), 7.93 (m, 1H), 8.02 (dm, J=7.0 Hz, 1H), 10.8 (s, 1H).

3-Trifluoromethanesulfonyloxy-[1,1′-biphenyl]-2,3′-dicarboxylic acid, dimethyl ester

To a solution of 3-hydroxy-[1,1′-biphenyl]-2,3′-dicarboxylic acid, dimethyl ester (1.1 g, 3.8 mmol) in dichloromethane (15 mL) were added pyridine (1.00 mL, 12.3 mmol) and trifluromethanesulfonic anhydride (0.90 mL, 5.4 mmol) at 0° C. The solution was stirred at 0° C. for 2 h. Aqueous 1 N HCl (20 mL) was added, and the mixture was extracted with dichloromethane (3×20 mL). The combined organic extracts were washed with water and brine, dried over MgSO₄, filtered, and concentrated to give 3-trifluoromethanesulfonyloxy-[1,1′-biphenyl]-2,3′-dicarboxylic acid, dimethyl ester (1.4 g, 87%) as a yellow solid: ¹H NMR (CDCl₃) δ 3.72 (s, 3H), 3.94 (s, 3H), 7.38-7.62 (m, 5H), 8.08 (m, 2H). 3-Ethenyl-[11 ′-biphenyl]-2,3′-dicarboxylic acid, dimethyl ester

A mixture of 3-trifluoromethanesulfonyloxy-[1,1′-biphenyl]-2,3′-dicarboxylic acid, dimethyl ester (1.3 g, 3.1 mmol), tetrakis(triphenylphosphine)palladium (0.36 g, 0.31 mmol), LiCl (0.94 g, 22.2 mmol), triethylamine (0.6 mL, 4.3 mmol) and tri-n-butyl(vinyl)tin (1.0 mL, 3.4 mmol) in 1,4-dioxane (30 mL) was heated at reflux under N₂ for 4 h. After cooling to rt, the mixture was filtered through a plug of silica gel and the filtrate was concentrated. Purification by flash chromatography (1:10 EtOAc/hexanes) provided 3-ethenyl-[1,1′-biphenyl]-2,3′-dicarboxylic acid, dimethyl ester (0.91 g, 99%) as a white solid: ¹H NMR (CDCl₃) δ 3.61 (s, 3H), 3.92 (s, 3H), 5.40 (d, J=11.1 Hz, 1H), 5.79 (d, J=17.5 Hz, 1H), 6.87 (dd, J=17.4, 11.0 Hz, 1H), 7.31 (d, J=7.5 Hz, 1H), 7.44-7.49 (m, 2H), 7.56 (dm, J=7.5 Hz, 1H), 7.62 (d, J=8.0 Hz, 1H), 8.03 (dm, J=7.5 Hz, 1H), 8.08 (t, 1, J=1.5 Hz, 1H).

3-[2-(Acetylthio)ethyl]-[1,1′-biphenyl]-2,3′-dicarboxylic acid, dimethyl ester

To a solution of 3-ethenyl-[1,1′-biphenyl]-2,3′-dicarboxylic acid, dimethyl ester (0.85 g, 2.9 mmol) in benzene (10 mL) was added thioacetic acid (2.1 mL, 29.4 mmol) followed by AIBN (0.053 g, 0.32 mmol). The solution was deoxygenated for 30 min by bubbling nitrogen through the solution and then heated at reflux for 4 h. Saturated aqueous NaHCO3 (20 mL) was added to the solution and the mixture was extracted with EtOAc (2×20 mL). The combined organic extracts were washed with water and brine, dried over MgSO₄, filtered, and concentrated. The residue was purified by flash chromatography (1:12 EtOAc/hexanes) to give 3-[2-(acetylthio)ethyl]-[1,1′-biphenyl]-2,3′-dicarboxylic acid, dimethyl ester (0.51 g, 48%) as an off white solid: ¹H NMR (CDCl₃) δ 2.35 (s, 3H), 2.93 (m, 2H), 3.14 (m, 2H), 3.62 (s, 3H), 3.93 (s, 3H), 7.29 (dd, J=7.6, 0.9 Hz, 1H), 7.35 (dd, J=7.5, 0.8 Hz, 1H), 7.44 (d, J=7.6 Hz, 1H), 7.48 (d, J=7.5 Hz, 1H), 7.55 (dt, J=8.0, 1.5 Hz, 1H), 8.03 (dt, J=7.9, 1.5 Hz, 1H), 8.07 (t, J=1.5 Hz, 1H).

3-(2-Mercaptoethyl)-[1,1′-biphenyl]-2,3′-dicarboxylic acid, 2-methyl ester

To a deoxygenated solution of 3-[2-(acetylthio)ethyl]-[1,1′-biphenyl]-2,3′-dicarboxylic acid, dimethyl ester (0.50 g, 1.34 mmol) in THF (3.5 mL) was added a deoxygenated solution of NaOH (0.38 g, 9.4 mmol) in water (3.5 mL). The mixture was stirred overnight, and 1 N HCl (20 mL) was added. The mixture was extracted with EtOAc (3×20 mL). The combined organic extracts were washed with water and brine, dried over MgSO₄, filtered, and concentrated to afford 3-(2-mercaptoethyl)-[1,1′-biphenyl]-2,3′-dicarboxylic acid, 2-methyl ester (0.35 g, 83%) as an off white solid: ¹H NMR (CDCl₃) δ 1.46 (t, J=8.0 Hz, 1H), 2.83 (m, 2H), 3.00 (m, 2H), 3.60 (s, 3H), 7.33-7.31 (m, 2H), 7.46 (t, J=7.7 Hz, 1H), 7.52 (t, J=7.7 Hz, 1H), 7.61 (dm, J=7.9 Hz, 1H), 8.10 (dm, J=7.9 Hz, 1H), 8.14 (m, 1H).

3-(2-Mercaptoethyl)-[1,1′-biphenyl]-2.3 ′-dicarboxylic acid

To a deoxygenated suspension of sodium ethanethiolate (0.135 g, 1.60 mmol) in DMF (0.5 mL) was added a solution of 3-(2-mercaptoethyl)-[1,1′-biphenyl]-2,3′-dicarboxylic acid, 2-methyl ester (0.10 g, 0.32 mmol) in DMF (0.5 mL). Argon was bubbled through the mixture for 10 min. The reaction was heated at 100° C. for 1 h and 200° C. for another hour. After the mixture cooled to rt, the reaction was quenched with 1 N HCl (20 mL) and was extracted with EtOAc (3×20 mL). The combined organic extracts were washed with water and brine, dried over MgSO₄, filtered, and concentrated to afford 3-(2-mercaptoethyl)-[1,1′-biphenyl]-2,3′-dicarboxylic acid (0.055 g, 57%) as a white solid: ¹H NMR (CDCl₃) δ 1.51 (t, J=8.0 Hz, 1H), 2.87-2.93 (m, 2H), 3.12-3.08 (m, 2H), 7.37 (m, 2H), 7.57-7.47 (m, 2H), 7.70 (dm, J=7.9 Hz, 1H), 7.98 (dm, J=7.8 Hz, 1H), 8.30 (m, 1H); ¹³C NMR (CDCl₃) δ 26.2, 38.8, 128.1, 129.3, 129.7, 129.8, 129.9 (2C), 130.5, 133.3, 134.4, 137.7, 139.1, 141.2, 172.3, 176.9. Elemental analysis calculated for C₁₆H₁₄O₄S: C, 63.56; H, 4.67; S, 10.61. Found: C, 63.65; H, 4.88; S, 10.33.

### Example 7

In Vitro Inhibition of NAALADase Activity

Various compounds used in the inventive methods and pharmaceutical compositions have been tested for in vitro inhibition of NAALADase activity. The experimental protocol and some of the results are set forth in U.S. Pat. Nos. 5,672,592, 5,795,877, 5,863,536, 5,880,112, 5,902,817, 5,962,521, 5,968,915, 6,025,344, 6,025,345, 6,028,216, 6,046,180, 6,054,444, 6,071,965, 6,121,252, 6,265,609, 6,348,464, 6,452,044, 6,458,775, 6,586,623, and International Publications Nos. WO 01/14390, WO 02/096866, WO 03/057670 and WO 02/092553, the entire contents of which patents and publications are herein incorporated by reference, as though set forth herein in full.

Other results are provided below in TABLES II and III.

TABLE II
IN VITRO INHIBITION OF NAALADASE ACTIVITY

| Compound | Kᵢ (nM) |
|---|---|
| 4-[4-(2,4-dicarboxybenzoyl)phenoxy]-1,2- | 1170 |
| benzenedicarboxylic acid | |
| 2-[(4-carboxyphenyl)sulfonyl]-1,4-benzenedicarboxylic | 2370 |
| acid | |
| 2-[(2,5-dicarboxyphenyl)sulfonyl]-1,4- | 1870 |
| benzenedicarboxylic acid | |
| 4-[(2-carboxyphenyl)thio]-1,3-benzenedicarboxylic acid | 3980 |
| 2-[(2-carboxyphenyl)thio]-1,4-benzenedicarboxylic acid | 572 |
| 4-[3-[[3-(2,4-dicarboxyphenoxy)-propyl]-dithio]propoxy]- | 3750 |
| 1,3-benzenedicarboxylic acid | |
| 5-(3-mercaptopropoxy)-1,3-benzenedicarboxylic acid | 3300 |
| 5-(2-mercaptoethoxy)-1,3-benzenedicarboxylic acid | 14500 |
| 5-[(hydroxyamino)-carbonyl]-1,3-benzenedicarboxylic | 1000 |
| acid | |
| 5-phosphono-1,3-benzenedicarboxylic acid | 14000 |
| 5-mercaptomethyl-1,3-benzenedicarboxylic acid | 6500 |
| 5-phosphonomethyl-1,3-benzenedicarboxylic acid | 3100 |
| 5-[(carboxymethyl)amino]-1,3-benzenedicarboxylic acid | 100000 |
| 5-[[(2-furanylmethyl)amino]methyl]-1,3- | 50000 |
| benzenedicarboxylic acid | |
| 2-carboxymethyl-1,4-benzenedicarboxylic acid | 9000 |
| 5-[2-(hydroxyamino)-2-oxoethyl]-1,3-benzenedicarboxylic | 12000 |
| acid | |
| 4-(2-mercaptoethyl)-1,3-benzenedicarboxylic acid | 116 |
| 5-(2-mercaptoethyl)-1,3-benzenedicarboxylic acid | 5100 |

TABLE III
IN VITRO INHIBITION OF NAALADASE ACTIVITY

| Compound | IC₅₀ |
|---|---|
| alpha-(3-mercaptopropyl)-3-(trifluoromethyl)- | 4000 |
| benzenepropanoic acid | |
| alpha-(3-mercaptopropyl)-benzenepropanoic acid | 2270 |
| 4-hydroxy-alpha-(3-mercaptopropyl)-benzenepropanoic | 2140 |
| acid | |
| 2,3,4,5,6-pentafluoro-alpha-(3-mercaptopropyl)- | 1000 |
| benzenepropanoic acid | |
| 3-carboxy-alpha-(3-mercaptopropyl)-benzenepropanoic | 17 |
| acid | |
| 4-carboxy-alpha-(3-mercaptopropyl)-benzenepropanoic | 59 |
| acid | |
| alpha-(3-mercaptopropyl)-4-(methylsulfonyl)- | 211 |
| benzenepropanoic acid | |
| 2-cyano-alpha-(3-mercaptopropyl)-benzenepropanoic acid | 450 |
| 5-(2-carboxy-5-mercaptopentyl)-1,3-benzenedicarboxylic | 2.55 |
| acid | |
| 5-carboxy-2-chloro-alpha-(3-mercaptopropyl)- | 2.09 |
| benzenepropanoic acid | |
| 3-carboxy-4-fluoro-alpha-(3-mercaptopropyl)- | 12 |
| benzenepropanoic acid | |
| 4-(2-cyanophenyl)-alpha-(3-mercaptopropyl)- | 316 |
| benzenepropanoic acid | |
| 2-(aminocarbonyl)-alpha-(3-mercaptopropyl)- | 3950 |
| benzenepropanoic acid | |
| 3-(1-carboxy-4-mercaptobutoxy)-benzoic acid | 16.3 |
| 5-mercapto-2-phenoxy-pentanoic acid | 555 |
| 2-(3,5-dimethoxyphenoxy)-5-mercapto-pentanoic acid | 16,100 |
| alpha-(3-mercaptopropyl)-2,5-dimethoxy- | 566 |
| benzenepropanoic acid | |
| alpha-(3-mercaptopropyl)-3-phenoxy-benzenepropanoic | 308 |
| acid | |
| 2-(3-hydroxyphenoxy)-5-mercapto-pentanoic acid | 846 |
| 3-(1-carboxy-4-mercaptobutoxy)-benzeneacetic acid | 64 |
| 4-(1-carboxy-4-mercaptobutoxy)-benzeneacetic acid | 82 |
| alpha-(3-mercaptopropyl)-4-phenyl-benzenepropanoic | 229 |
| acid | |
| 2-(3-acetylphenoxy)-5-mercapto-pentanoic acid | 2900 |
| 2-[3-(acetylamino)phenoxy]-5-mercapto-pentanoic acid | 27700 |
| 2-(4-acetylphenoxy)-5-mercaptopentanoic acid | 29.8 |
| 4-(acetylamino)-alpha-(3-mercaptopropyl)- | 2200 |
| benzenepropanoic acid | |
| 3-(1-carboxy-4-mercaptobutoxy)-4-methoxy-benzoic acid | 287 |
| 4-(carboxymethyl)-alpha-(3-mercaptopropyl)- | 809 |
| benzenepropanoic acid | |
| 2-(1-carboxy-4-mercaptobutoxy)-benzoic acid | 4600 |
| 4-(1-carboxy-4-mercaptobutoxy)-benzoic acid | 192 |
| 3-carboxy-2-chloro-alpha-(3-mercaptopropyl)- | 1200 |
| benzenepropanoic acid | |
| 3-carboxy-4-chloro-alpha-(3-mercaptopropyl)- | 175 |
| benzenepropanoic acid | |
| 3-(1-carboxy-4-mercaptobutoxy)-4-chloro-benzoic acid | 118 |
| 3-(1-carboxy-4-mercaptobutoxy)-4-fluoro-benzoic acid | 400 |
| 5-carboxy-2-fluoro-alpha-(3-mercaptopropyl)- | 6.33 |
| benzenepropanoic acid | |
| 5-carboxy-alpha-(3-mercaptopropyl)-2-methoxy- | 26 |
| benzenepropanoic acid | |
| 4-carboxy-alpha-(3-mercaptopropyl)-1- | 316 |
| naphthalenepropanoic acid | |
| 2-carboxy-alpha-(3-mercaptopropyl)-benzenepropanoic | 1700 |
| acid | |
| 4-carboxy-2,3,5,6-tetrafluoro-alpha-(3-mercaptopropyl)- | 660 |
| benzenepropanoic acid | |
| 5-mercapto-2-(phenylthio)-pentanoic acid | 297 |
| 3-[1-carboxy-4-mercaptobutyl)thio]-benzoic acid | 24 |
| alpha-(3-mercaptopropyl)-2-naphthalenepropanoic acid | 1130 |
| 2-chloro-alpha-(3-mercaptopropyl)-benzenepropanoic | 1350 |
| acid | |
| alpha-(3-mercaptopropyl)-3- | 1130 |
| [[(phenylmethyl)amino]carbonyl]-benzenepropanoic acid | |
| 3-bromo-5-carboxy-alpha-(3-mercaptopropyl)- | 2.5 |
| benzenepropanoic acid | |
| 3-[[(carboxymethyl)amino]carbonyl]-alpha-(3- | 286 |
| mercaptopropyl)-benzenepropanoic acid | |
| 3-bromo-4-carboxy-alpha-(3-mercaptopropyl)- | 146 |
| benzenepropanoic acid | |
| 3-carboxy-alpha-(3-mercaptopropyl)-5-nitro- | 3 |
| benzenepropanoic acid | |
| 3-carboxy-5-(1,1-dimethylethyl)-alpha-(3- | 0.05 |
| mercaptopropyl)-benzenepropanoic acid | |
| 5-carboxy-alpha-(3-mercaptopropyl)-2-nitro- | 8.25 |
| benzenepropanoic acid | |
| 3′-(2-carboxy-5-mercaptopentyl)-[1,1′-biphenyl]-3- | 1.83 |
| carboxylic acid | |
| 2-bromo-5-carboxy-alpha-(3-mercaptopropyl)- | 3.33 |
| benzenepropanoic acid | |
| (+)-3-carboxy-alpha-(3-mercaptopropyl)- | 7 |
| benzenepropanoic acid | |
| (−)-3-carboxy-alpha-(3-mercaptopropyl)- | 33.3 |
| benzenepropanoic acid | |
| 2-(2-carboxy-5-mercaptopentyl)-[1,1′-biphenyl]-4- | 19 |
| carboxylic acid | |
| 6-(2-carboxy-5-mercaptopentyl)-[1,1′-biphenyl]-2- | 70 |
| carboxylic acid | |
| 4-(2-carboxy-5-mercaptopentyl)-[1,1′-biphenyl]-2- | 18 |
| carboxylic acid | |
| 3-carboxy-alpha-(3-mercaptopropyl)-5-methoxy- | 13 |
| benzenepropanoic acid | |
| 3′-(2-carboxy-5-mercaptopentyl)-[1,1′-biphenyl]-2- | 184 |
| carboxylic acid | |
| 3-(2-carboxyphenoxy)-alpha-(3-mercaptopropyl)- | 49 |
| benzenepropanoic acid | |
| 4-(2-carboxyphenoxy)-alpha-(3-mercaptopropyl)- | 3590 |
| benzenepropanoic acid | |
| 3-carboxy-alpha-(3-mercaptobutyl)-benzenepropanoic | 30 |
| acid | |
| 4′-(2-carboxy-5-mercaptopropyl)-[1,1′-biphenyl]-2- | 87 |
| carboxylic acid | |
| alpha-(3-mercaptopropyl)-3-phenyl-benzenepropanoic | 181 |
| acid | |
| 3-carboxy-alpha-(3-mercaptopropyl)-5-phenoxy- | 5 |
| benzenepropanoic acid | |
| 3-carboxy-5-(1,1-dimethylethyl)-alpha-(3-mercaptobutyl)- | 1 |
| benzenepropanoic acid | |
| 2-(2-mercaptoethyl)-benzoic acid | 613 |
| 5-hydroxy-2-(2-mercaptoethyl)-benzoic acid | 9170 |
| 5-[(4-carboxyphenyl)methoxy]-2-(2-mercaptoethyl)- | 71.5 |
| benzoic acid | |
| 2-(2-mercaptoethyl)-5-(phenylmethoxy)-benzoic acid, | 380 |
| 2-(carboxymethoxy)-6-(2-mercaptoethyl)-benzoic acid | 215 |
| 5-[(3-carboxyphenyl)methoxy]-2-(2-mercaptoethyl)- | 84.5 |
| benzoic acid | |
| 2-(2-mercaptoethyl)-6-(phenylmethoxy)-benzoic acid | 11.5 |
| 2-[(2-carboxyphenyl)methoxy]-6-(2-mercaptoethyl)- | 11.5 |
| benzoic acid | |
| 2-[(4-carboxyphenyl)methoxy]-6-(2-mercaptoethyl)- | 4 |
| benzoic acid | |
| 3-(2-mercaptoethyl)-[1,1′-biphenyl]-2,3′-dicarboxylic acid | 3.46 |
| 5-(mercaptomethyl)-2-(2-phenylethoxy)-benzoic acid | 188 |
| 2-(3,3-dimethylbutoxy)-6-(2-mercaptoethyl)-benzoic acid | 2580 |
| 2-(2-mercaptoethyl)-6-(2-phenylethoxy)-benzoic acid | 266 |
| 2-[(2-chlorophenyl)methoxy]-6-(2-mercaptoethyl)-benzoic | 160 |
| acid | |
| 2-[[3-carboxy-5-(1,1-dimethylethyl)phenyl]methoxy]-6-(2- | 23.7 |
| mercaptoethyl)-benzoic acid | |
| 3-(2-mercaptoethyl)-[1,1′-biphenyl]-2,4′-dicarboxylic acid | 4.28 |
| 2-[(4-carboxy-2-methoxyphenyl)methoxy]-6-(2- | 7 |
| mercaptoethyl)-benzoic acid | |
| 2-[(4-carboxy-3-methoxyphenyl)methoxy]-6-(2- | 10.5 |
| mercaptoethyl)-benzoic acid | |
| 2-[(2-bromo-4-carboxyphenyl)methoxy]-6-(2- | 2.65 |
| mercaptoethyl)-benzoic acid | |
| 2-[(3-bromo-4-carboxyphenyl)methoxy]-6-(2- | 0.098 |
| mercaptoethyl)-benzoic acid | |
| 2-(2-mercaptoethyl)-6-phenoxy-benzoic acid | 1150 |
| 4-(mercaptomethyl)-[1,1′-biphenyl]-2,3′-dicarboxylic acid | 76 |
| 5-(mercaptomethyl)-2-(phenylmethoxy)-benzoic acid | 85 |
| 4-bromo-3-(mercaptomethyl)-benzoic acid | 3200 |
| 3-(2-mercaptoethyl)-benzoic acid | 6050 |
| 3-(mercaptomethyl)-benzoic acid | 3780 |
| 2-(mercaptomethyl)-benzoic acid | 100000 |
| 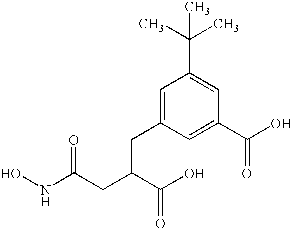 3-tert-Butyl-5-(2-carboxy-3-hydroxycarbamoyl-propyl)- benzoic acid | 296.3 |
| 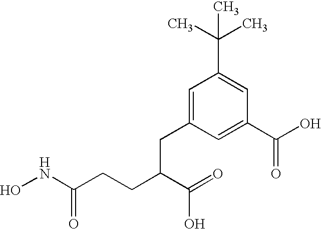 3-tert-Butyl-5-(2-carboxy-4-hydroxycarbamoyl-butyl)- benzoic acid | 0.2 |
| 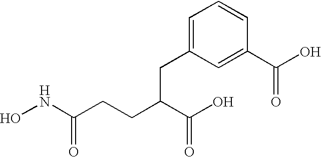 3-(2-Carboxy-4-hydroxycarbamoyl-butyl)-benzoic acid | 47.8 |
| 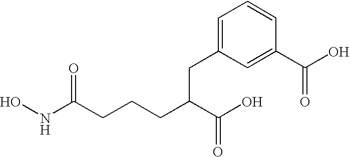 3-(2-Carboxy-5-hydroxycarbamoyl-pentyl)-benzoic acid | 66.3 |
| 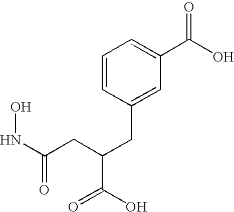 3-(2-Carboxy-3-hydroxycarbamoyl-propyl)-benzoic acid | 693 |
| 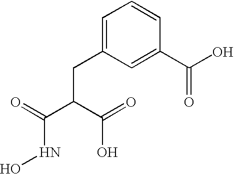 3-(2-Carboxy-2-hydroxycarbamoyl-ethyl)-benzoic acid | 4530 |
| 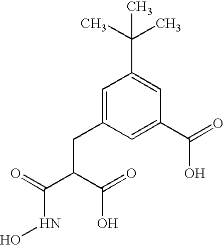 3-tert-Butyl-5-(2-carboxy-2-hydroxycarbamoyl-ethyl)- benzoic acid | 11900 |
| 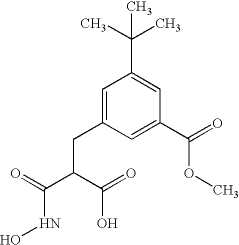 3-tert-Butyl-5-(2-carboxy-2-hydroxycarbamoyl-ethyl)- benzoic acid methyl ester | 38600 |
| 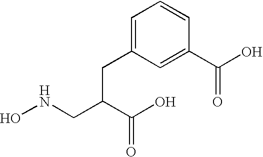 3-(2-Carboxy-3-hydroxyamino-propyl)-benzoic acid | 44000 |
| 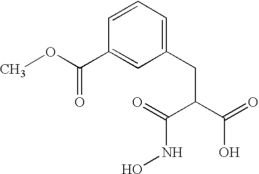 3-(2-Carboxy-2-hydroxycarbamoyl-ethyl)-benzoic acid methyl ester | 100000 |
| 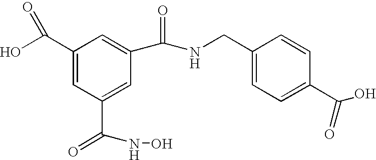 | 2780 |
| 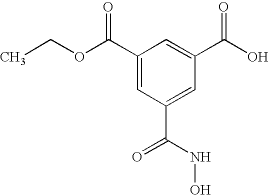 5-Hydroxycarbamoyl-isophthalic acid monoethyl ester | 29000 |
| 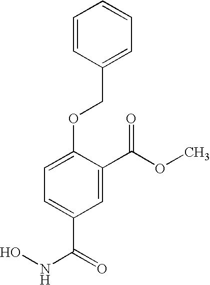 6-Benzyloxy-N-hydroxy-isophthalamic acid methyl ester | 100000 |
| 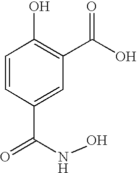 6,N-Dihydroxy-isophthalamic acid | 100000 |
| 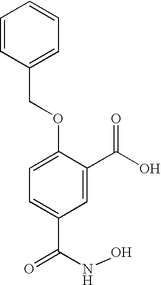 6-Benzyloxy-N-hydroxy-isophthalamic acid | 100000 |
| 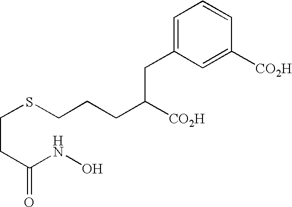 3-[2-Carboxy-5-(2-hydroxycarbamoyl-ethylsulfanyl)- pentyl]-benzoic acid | 113 |
| 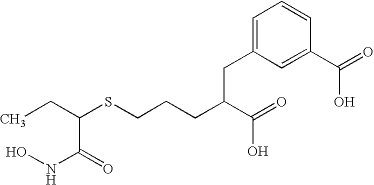 3-[2-Carboxy-5-(1-hydroxycarbamoyl-propylsulfanyl)- pentyl]-benzoic acid | 3291 |
| 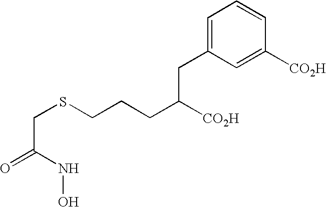 3-(2-Carboxy-5-hydroxycarbamoylmethyl-sulfanylpentyl)- benzoic acid | 384 |
| 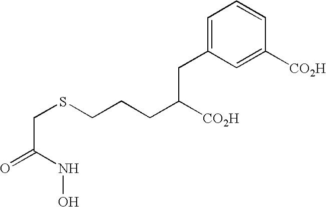 3-(2-Carboxy-5-hydroxycarbamoylmethyl-sulfanyl-pentyl)- benzoic acid | 394 |
| 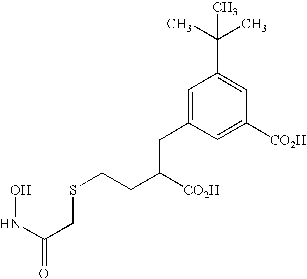 3-tert-Butyl-5-(2-carboxy-4-hydroxycarbamoylmethyl- sulfanylbutyl)-benzoic acid | 1060 |
| 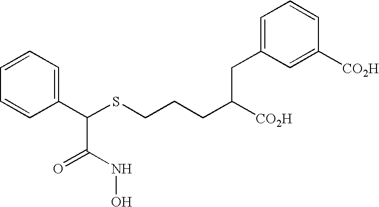 3-[2-Carboxy-5-(hydroxycarbamoyl-phenyl- methylsulfanyl)-pentyl]-benzoic acid | 1120 |
| 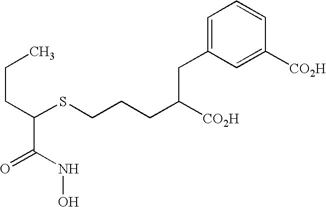 3-[2-Carboxy-5-(1-hydroxycarbamoyl-butylsulfanyl)- pentyl]-benzoic acid | 1530 |
| 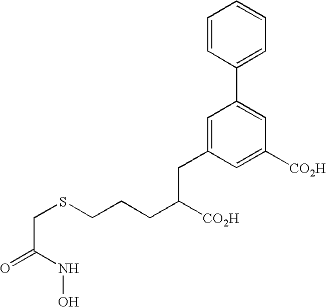 5-(2-Carboxy-5-hydroxycarbamoylmethyl-sulfanylpentyl)- biphenyl-3-carboxylic acid | 1690 |
| 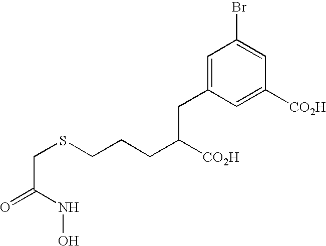 3-Bromo-5-(2-carboxy-5-hydroxycarbamoyl- methylsulfanylpentyl)-benzoic acid | 2460 |
| 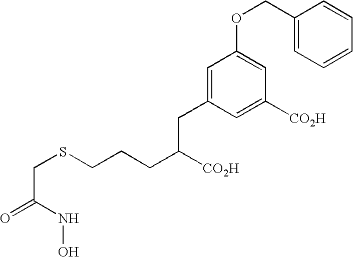 3-Benzyloxy-5-(2-carboxy-5-hydroxycarbamoylmethyl- sulfanylpentyl)-benzoic acid | 2570 |
| 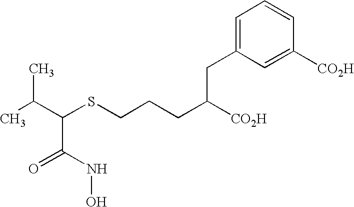 3-[2-Carboxy-5-(1-hydroxycarbamoyl-2-methyl- propylsulfanyl)-pentyl]-benzoic acid | 2800 |
| 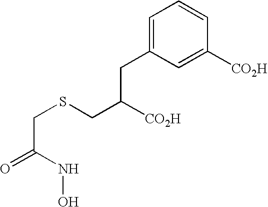 3-(2-Carboxy-3-hydroxycarbamoylmethyl-sulfanylpropyl)- benzoic acid | 3070 |
| 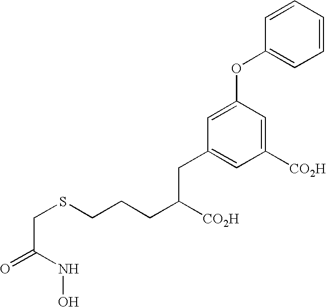 3-(2-Carboxy-5-hydroxycarbamoylmethyl-sulfanylpentyl)- 5-phenoxy-benzoic acid | 3250 |
| 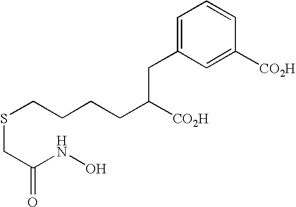 3-(2-Carboxy-6-hydroxycarbamoylmethyl-sulfanylhexyl)- benzoic acid | 3330 |
| 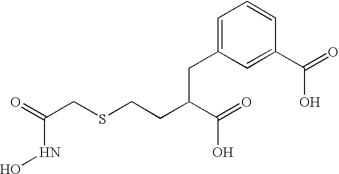 3-(2-Carboxy-4-hydroxycarbamoylmethyl-sulfanylbutyl)- benzoic acid | 4900 |
| 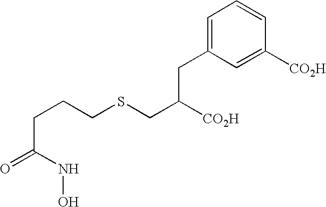 3-[2-Carboxy-3-(3-hydroxycarbamoyl-propylsulfanyl)- propyl]-benzoic acid | 6050 |
| 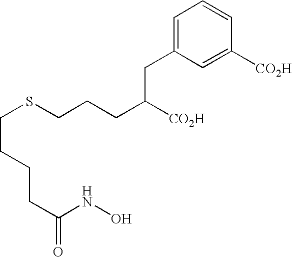 3-[2-Carboxy-5-(4-hydroxycarbamoyl-butylsulfanyl)- pentyl]-benzoic acid | 7580 |
| 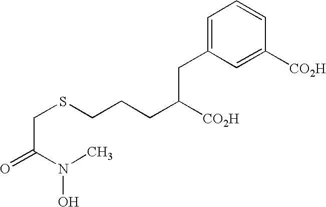 3-{2-Carboxy-5-[(hydroxy-methyl-carbamoyl)- methylsulfanyl]-pentyl}-benzoic acid | 14900 |
| 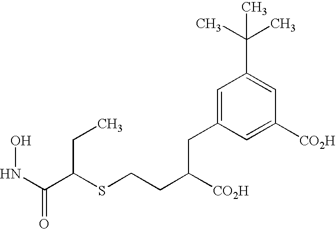 3-tert-Butyl-5-[2-carboxy-4-(1-hydroxycarbamoyl- propylsulfanyl)-butyl]-benzoic acid | 16000 |
| 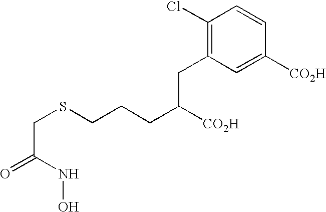 3-(2-Carboxy-5-hydroxycarbamoylmethyl-sulfanylpentyl)- 4-chloro-benzoic acid | 16000 |
| 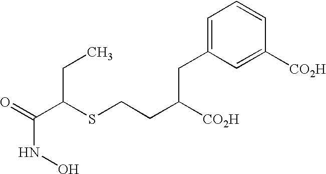 3-[2-Carboxy-4-(1-hydroxycarbamoyl-propylsulfanyl)- butyl]-benzoic acid | 19000 |
| 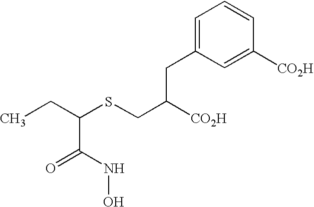 3-[2-Carboxy-3-(1-hydroxycarbamoyl-propylsulfanyl)- propyl]-benzoic acid | 41500 |
| 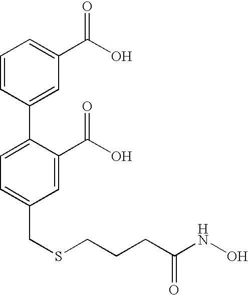 4-(3-Hydroxycarbamoyl-propylsulfanylmethyl)-biphenyl- 2,3′-dicarboxylic acid | 10820 |
| 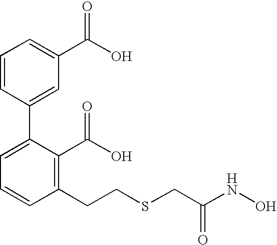 3-(2-1-Hydroxycarbamoyl-methylsulfanyl-ethyl)-biphenyl- 2,3′-dicarboxylic acid | 255 |
| 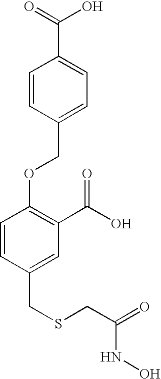 | 1330 |
| 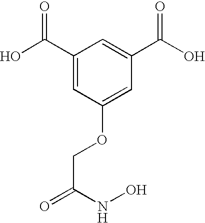 5-Hydroxycarbamoylmethoxy-isophthalic acid | 14800 |
| 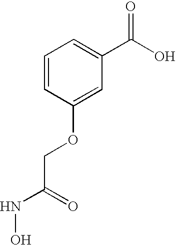 3-Hydroxycarbamoylmethoxy-benzoic acid | 40800 |
| 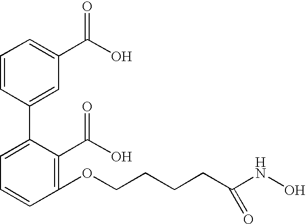 3-(4-Hydroxycarbamoyl-butoxy)-biphenyl-2,3′- dicarboxylic acid | 55.9 |
| 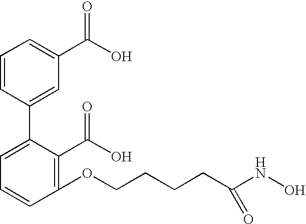 3-(4-Hydroxycarbamoyl-butoxy)-biphenyl-2,3′- dicarboxylic acid | 4800 |
| 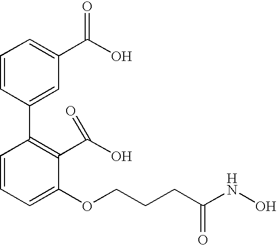 3 -(3-Hydroxycarbamoyl-propoxy)-biphenyl-2,3′- dicarboxylic acid | 9100 |
| 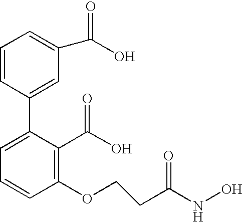 3-(2-1-Hydroxycarbamoyl-ethoxy)-biphenyl-2,3′-dicarboxylic acid | 14900 |
| 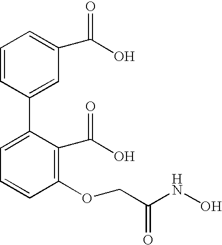 3-Hydroxycarbamoylmethoxy-biphenyl-2,3′-dicarboxylic acid | 27600 |
| 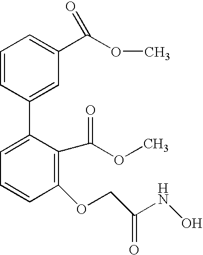 3-Hydroxycarbamoylmethoxy-biphenyl-2,3′-dicarboxylic acid dimethyl ester | 100000 |
| 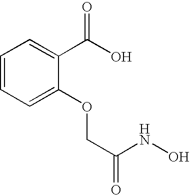 2-Hydroxycarbamoylmethoxy-benzoic acid | 100000 |
| 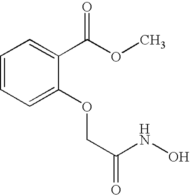 2-Hydroxycarbamoylmethoxy-benzoic acid methyl ester | 100000 |
| 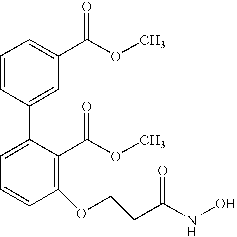 3-(2-Hydroxycarbamoyl-ethoxy)-biphenyl-2,3′-dicarboxylic acid dimethyl ester | 100000 |
| 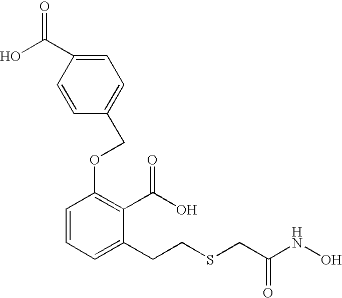 | 14000 |
| 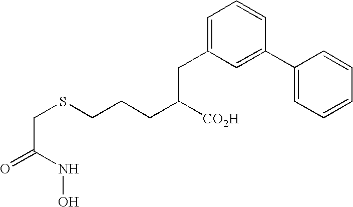 2-Biphenyl-3-ylmethyl-5-hydroxy- carbamoylmethylsulfanyl-pentanoic acid | 4210 |
| 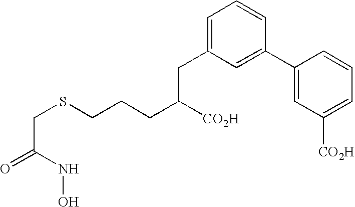 3′-(2-Carboxy-5-hydroxycarbamoylmethylsulfanyl-pentyl)- biphenyl-3-carboxylic acid | 23500 |
| 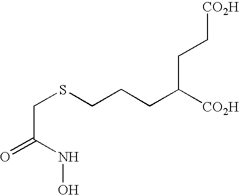 2-(3-Hydroxycarbamoyl-methylsulfanylpropyl)- pentanedioic acid | 3000 |
| 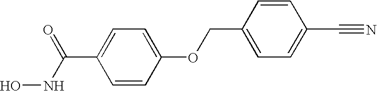 4-(4-Cyano-benzyloxy)-N-hydroxy-benzamide | 14150 |
| 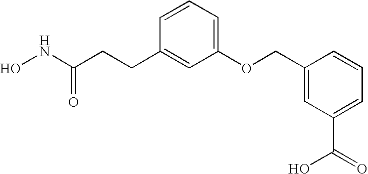 3-[3-(2-Hydroxycarbamoyl-ethyl)-phenoxymethyl]-benzoic acid | 92675 |
| 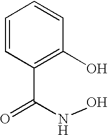 2,N-Dihydroxy-benzamide | 100000 |
| 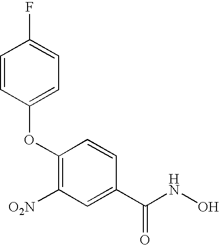 4-(4-Fluoro-phenoxy)-N-hydroxy-3-nitro-benzamide | 100000 |
| 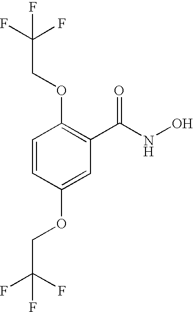 N-Hydroxy-2,5-bis-(2,2,2-trifluoro-ethoxy)-benzamide | 100000 |
| 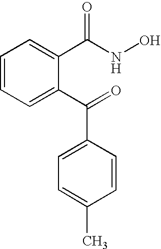 N-Hydroxy-2-(4-methyl-benzoyl)-benzamide | 100000 |
| 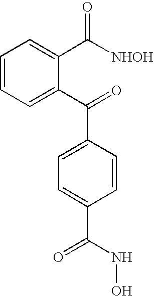 | 100000 |

### Example 8

Effects of NAALADase Inhibitors on Morphine Tolerance (Experiment 1) and Acute Effects in the Tail-Flick Test (Experiment 2)

Experimental Protocol

Subjects

Male C57/BL mice (IMP, Lodz, Poland), 22-24 g of body weight were group-housed in the standard laboratory cages and kept in a temperature-controlled colony room (21+2° C.) with a 12-hr light/dark cycle (light on: 07:00, off: 19:00). Commercial food and tap water were available ad libitum. Each experimental group consisted of 7-28 mice per treatment. All mice were used only once.

Apparatus for Experiments 1-2

A standardized tail-flick analgesia meter (Columbus, Ohio, USA, model 33), adjusted to sensitivity of “10” with radiant heat source and connected to an automatic timer was used to assess antinociceptive responses. The intensity of the heat stimulus was adjusted so that the baseline tail-flick latency was ˜3 s. A maximum latency of 10 s (i.e., cut-off) was used to minimize damage to the tail. The tail withdrawal latency was measured from the start of heat stimulus until the mouse exhibited a flick of the tail. Each response assessment consisted of two separate measurements taken at different portions of the tail (spaced by 1.5-2 cm) and separated by 15 s. The mean of these responses was used for subsequent comparisons.

Morphine antinociceptive potency was investigated with the use of cumulative dose-response curves that allowed for minimization of the animal number used (Paronis and Holtzman 1991). After adaptation and baseline trials, each mouse was injected s.c. with a low dose of morphine (1 mg/kg). Thirty min later, the mouse was retested and injected with the next dose of morphine that was increased by quarter of a log unit. Thus, because the initial dose of morphine was 1.0 mg/kg, the next dose was 1.78 mg/kg, for a cumulative dose of 2.8 mg/kg. This procedure continued until either the mouse did not move his tail within the cut-off time or until there was a plateauing of the dose-response curve, so that the latency did not increase from one dose to the next. Each analgesic responder was not subjected to further tail flick assessments but was injected with the subsequent dose of morphine so that every animal received the same total dose of morphine during a given test.

Effects on Morphine Tolerance (Experiment 1) and Acute Effects in the Tail-Flick Test (Experiment 2)

Experiment 1 was carried out to investigate the effect of 2-PMPA on the development of morphine tolerance. On day 1 (test #1), the first measurement of morphine antinociceptive potency was performed, followed by 6 days of bid morphine injections (10 mg/kg, s.c., 7:30 and 17:30) (Elliott et al. 1994; Popik et al. 2000b). Pretreatment with 2-PMPA (30, 50 or 100 mg/kg, i.p.) or memantine (7.5 mg/kg, s.c., a “positive control”) was given at 30 min prior to each morphine dose on days 2-7. On day 8 (test #2), the second measurement of morphine antinociceptive potency was carried out. The degree of morphine tolerance was assessed by comparing the morphine antinociceptive potencies (cumulative dose-response curves) obtained in tests #1 and #2.

Experiment 2 was designed to determine whether 2-PMPA might itself produce antinociceptive effects and/or affect the antinociceptive effects of morphine. Morphine (1.5 or 3 mg/kg, s.c.) was administered 30 min after injection of 100 mg/kg of 2-PMPA or placebo, administered i.p. The 3 mg/kg dose of morphine corresponds to the antinociceptive ED₅₀ dose in these test conditions (data not shown).

Results

Effects of 2-PMPA on Development of Morphine Tolerance (Experiment 1)

There were no differences in antinociceptive morphine ED₅₀ values on test #1 among groups (Table IV). Treatment with 10 mg/kg bid of morphine produced 6.44 fold increase in the ED₅₀ values as determined on test #2. In contrast, pretreatment with memantine, 50 or 100 (but not 30) mg/kg of 2-PMPA given prior to each dose of morphine attenuated the development of morphine tolerance. The effects of 2-PMPA were related to the dose. This was evidenced by a significant decrease in both test #2 ED₅₀ values (statistically significant for the dose 100 mg/kg) and antinociceptive morphine fold shifts of 2-PMPA for the doses of 100 and 50 mg/kg, as compared with the control group that received placebo+morphine (Table IV). Similarly, memantine (7.5 mg/kg) produced an inhibition of morphine tolerance.

TABLE IV
Effects of NAALADase inhibitor and memantine on the
development of tolerance to morphine

| Treatment/dose | | | |
|---|---|---|---|
| mg/kg (N) | Test #1 ED₅₀ | Test #2 ED₅₀ | Fold Shift |
| Placebo + Morphine (8) | 1.49 ± 0.26 | 8.85 ± 2.22 | 6.44 ± 1.17 |
| Placebo + Placebo (8) | 2.23 ± 0.42 | 3.28 ± 0.47* | 1.70 ± 0.29* |
| 2-PMPA 30 + | 2.00 ± 0.43 | 9.47 ± 2.13 | 5.20 ± 1.26 |
| Morphine (9) | | | |
| 2-PMPA 50 + | 1.87 ± 0.34 | 5.41 ± 1.11 | 3.20 ± 0.66* |
| Morphine (9) | | | |
| 2-PMPA 100 + | 1.59 ± 0.30 | 3.49 ± 0.83* | 2.70 ± 0.57* |
| Morphine (10) | | | |
| Memantine 7.5 + | 1.51 ± 0.29 | 3.52 ± 0.88* | 2.60 ± 0.49* |
| Morphine (8) | | | |
| ANOVA: F(5,46) = | 0.71; ns | 3.891; P < 0.01 | 4.555; P < 0.01 |

Presented are mean ED₅₀ values with ±SEM determined during test #1 (pre-morphine) and test #2 (post-morphine) as well as resulting fold shifts. Asterisks (*) indicate a statistically significant difference compared to the Placebo+Morphine group that received saline and morphine during the development of morphine tolerance (*p<0.05, Newman Keul's test).

Effects of 2-PMPA on the Tail-flick Response and Antinociceptive Effects of Morphine (Experiment 2)

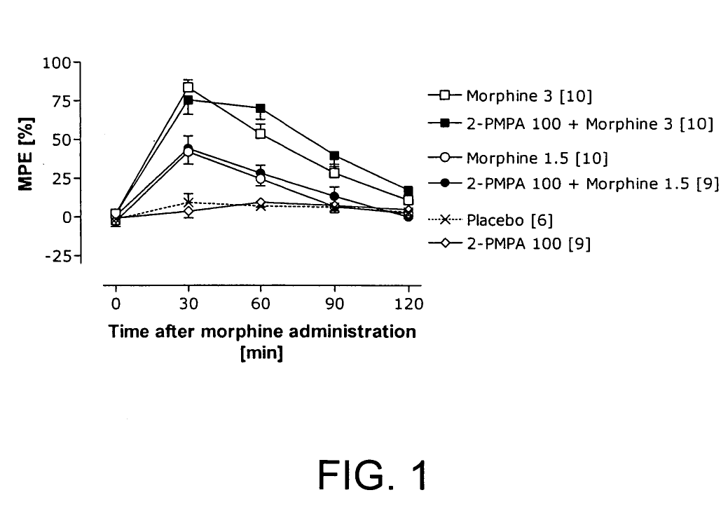

Analysis of areas under curve (AUC) revealed that treatment with placebo+1.5 and 3 mg/kg of morphine produced statistically significantly longer tail-flick responses compared to placebo+placebo treatment. In contrast, 100 mg/kg of 2-PMPA+placebo treatment did not affect tail-flick responses as compared to placebo+placebo treatment. Moreover, this dose of 2-PMPA did not affect antinociceptive effects of 1.5 or 3 mg/kg of morphine (FIG. 1).

Presented in FIG. 1 are the time courses of tail-flick responses of mice treated with combination of 2-PMPA and morphine. The N is given in brackets. Inset: Presented are mean ±S.E.M. Area Under Curve (AUC) values calculated on the same data. One way ANOVA F(5,48)=19.28, P<0.0001 and post-hoc Newman-Keul's test revealed that the treatment with placebo+morphine 1.5 mg/kg and with 100 mg/kg 2-PMPA+morphine 1.5 mg/kg differed significantly (**, P<0.01) from placebo+placebo treatment. Similarly, treatment with placebo+morphine 3 mg/kg and that with 100 mg/kg 2-PMPA+morphine 3 mg/kg differed significantly (***, P<0.001) from placebo+placebo treatment. Effects of 100 mg/kg of 2-PMPA+placebo treatment did not differ from placebo+placebo treatment. Effects of placebo+respective doses of morphine did not differ from the effects of 2-PMPA+respective doses of morphine.

All publications, patents and patent applications identified above are herein incorporated by reference.

The invention being thus described, it will be apparent to those skilled in the art that the same may be varied in many ways without departing from the spirit and scope of the invention. Such variations are included within the scope of the invention to be claimed.

We claim:

1. A method for treating opioid tolerance comprising administering an effective amount of a NAALADase inhibitor to a mammal in need of such treatment.

2. The method of claim 1, wherein the NAALADase inhibitor is an acid containing a metal binding group.

3. The method of claim 1, wherein the NAALADase inhibitor is a compound of formula I 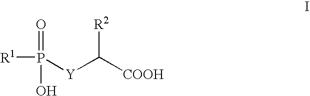
or an enantiomer or a pharmaceutically acceptable equivalent of said compound, wherein:
Y is CR³R⁴, NR⁵ or O;
R¹ is hydrogen, C₁-C₉ alkyl, C₂-C₉ alkenyl, C₃-C₈ cycloalkyl, C₅-C₇ cycloalkenyl, Ar, COOR⁶, NR⁶R⁷ or OR⁶, wherein said alkyl, alkenyl, cycloalkyl and cycloalkenyl are independently unsubstituted or substituted with one or more substituent(s) which are, for example, independently selected from carboxy, C₃-C₈ cycloalkyl, C₅-C₇ cycloalkenyl, halo, hydroxy, nitro, trifluoromethyl, C₁-C₆ alkyl, C₂-C₆ alkenyl, C₁-C₉ alkoxy, C₂-C₉ alkenyloxy, phenoxy, benzyloxy, COOR⁶, NR⁶R⁷ and Ar;
R² is hydrogen, C₁-C₆ alkyl, C₂-C₆ alkenyl, C₃-C₈ cycloalkyl, C₅-C₇ cycloalkenyl, Ar, halo or carboxy, wherein said alkyl, alkenyl, cycloalkyl and cycloalkenyl are independently unsubstituted or substituted with one or more substituent(s) which are, for example, independently selected from carboxy, C₃-C₈ cycloalkyl, C₅-C₇ cycloalkenyl, halo, hydroxy, nitro, trifluoromethyl, C₁-C₆ alkyl, C₂-C₆ alkenyl, C₁-C₉ alkoxy, C₂-C₉ alkenyloxy, phenoxy, benzyloxy, NR⁶R⁷ and Ar;
R³ and R⁴ are independently hydrogen or C₁-C₃ alkyl;
R⁵ is hydrogen or C₁-C₃ alkyl;
R⁶ and R⁷ are independently hydrogen, C₁-C₉ alkyl, C₂-C₉ alkenyl, C₃-C₈ cycloalkyl, C₅-C₇ cycloalkenyl or Ar, wherein said alkyl, alkenyl, cycloalkyl and cycloalkenyl are independently unsubstituted or substituted with one or more substituent(s) which are, for example, independently selected from carboxy, C₃-C₈ cycloalkyl, C₅-C₇ cycloalkenyl, halo, hydroxy, nitro, trifluoromethyl, C₁-C₆ alkyl, C₂-C₆ alkenyl, C₁-C₉ alkoxy, C₂-C₉ alkenyloxy, phenoxy, benzyloxy and Ar; and
Ar is selected from 1-naphthyl, 2-naphthyl, 2-indolyl, 3-indolyl, 4-indolyl, 2-furyl, 3-furyl, tetrahydrofuranyl, tetrahydropyranyl, 2-thienyl, 3-thienyl, 2-pyridyl, 3-pyridyl, 4-pyridyl and phenyl, wherein said Ar is unsubstituted or substituted with one or more substituent(s) which are, for example, independently selected from halo, hydroxy, nitro, trifluoromethyl, C₁-C₆ alkyl, C₂-C₆ alkenyl, C₁-C₆ alkoxy, C₂-C₆ alkenyloxy, phenoxy, benzyloxy, carboxy and N⁶R⁷.

4. The method of claim 3, wherein Y is CH₂.

5. The method of claim 4, wherein R² is —(CH₂)₂COOH.

6. The method of claim 5, wherein R¹ is hydrogen, C₁-C₄ alkyl, C₂-C₄ alkenyl, C₃-C₈ cycloalkyl, C₅-C₇ cycloalkenyl, benzyl, phenyl or OR⁶, wherein said alkyl, alkenyl, cycloalkyl, cycloalkenyl, benzyl and phenyl are independently unsubstituted or substituted with one or more substituent(s) independently selected from carboxy, C₃-C₈ cycloalkyl, C₅-C₇ cycloalkenyl, halo, hydroxy, nitro, trifluoromethyl, C₁-C₆ alkyl, C₂-C₆ alkenyl, C₁-C₆ alkoxy, C₂-C₆ alkenyloxy, phenoxy, benzyloxy, NR⁶R⁷, benzyl and phenyl.

7. The method of claim 6, wherein the compound of formula I is selected from:
2-(phosphonomethyl)pentanedioic acid;
2-[[(2-carboxyethyl)hydroxyphosphinyl]methyl]-pentanedioic acid;
2-[(benzylhydroxyphosphinyl)methyl]pentanedioic acid;
2-[(phenylhydroxyphosphinyl)methyl]pentanedioic acid;
2-[[((hydroxy)phenylmethyl)hydroxyphosphinyl]-methyl]pentanedioic acid;
2-[(butylhydroxyphosphinyl)methyl]pentanedioic acid;
2-[[(3-methylbenzyl)hydroxyphosphinyl]methyl]-pentanedioic acid;
2-[(3-phenylpropylhydroxyphosphinyl)methyl]-pentanedioic acid;
2-[[(4-fluorophenyl)hydroxyphosphinyl]methyl]-pentanedioic acid;
2-[(methylhydroxyphosphinyl)methyl]pentanedioic acid;
2-[(phenylethylhydroxyphosphinyl)methyl]pentanedioic acid;
2-[[(4-methylbenzyl)hydroxyphosphinyl]methyl]-pentanedioic acid;
2-[[(4-fluorobenzyl)hydroxyphosphinyl]methyl]-pentanedioic acid;
2-[[(4-methoxybenzyl)hydroxyphosphinyl]methyl]-pentanedioic acid;
2-[[(3-trifluoromethylbenzyl)hydroxyphosphinyl]-methyl]pentanedioic acid;
2-[[4-trifluoromethylbenzyl)hydroxyphosphinyl]-methyl]pentanedioic acid;
2-[[(2-fluorobenzyl)hydroxyphosphinyl]methyl]-pentanedioic acid;
2-[[(2,3,4,5,6-pentafluorobenzyl)hydroxy-phosphinyl]methyl]pentanedioic acid; and
enantiomers and pharmaceutically acceptable equivalents.

8. The method of claim 1, wherein the NAALADase inhibitor is a compound of formula II 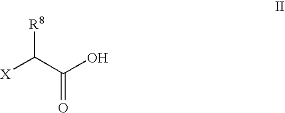
or an enantiomer or a pharmaceutically acceptable equivalent of said compound, wherein:
X is a moiety of formula III, IV or V 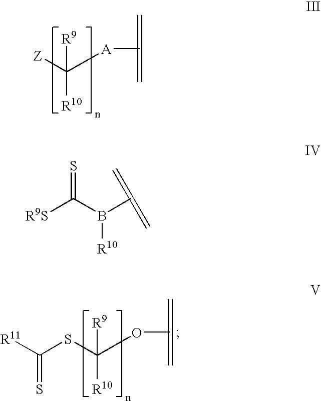
Z is SH, SO_{3H}, SO₂H , SOH, SO(NH)R¹² or S(NHR¹²)₂R¹³;
B is N or CR¹⁴;
A is O, S, CR¹⁵R¹⁶ or (CR¹⁵R¹⁶)ₘS;
m and n are independently 0, 1, 2, 3 or 4;
R⁸, R⁹, R¹⁰, R¹¹, R¹², R¹⁴, R¹⁵ and R¹⁶ are independently hydrogen, C₁-C₉ alkyl, C₂-C₉ alkenyl, C₃-C₈ cycloalkyl, C₅-C₇ cycloalkenyl, Ar¹, hydroxy, carboxy, carbonyl, amino, cyano, isocyano, nitro, sulfonyl, sulfoxy, thio, thiocarbonyl, thiocyano, formanilido, thioformamido, sulfhydryl, halo, haloalkyl, trifluoromethyl or oxy, wherein said alkyl, alkenyl, cycloalkyl and cycloalkenyl are independently unsubstituted or substituted with one or more substituent(s); and
Ar¹ is a carbocyclic or heterocyclic moiety, which is unsubstituted or substituted with one or more substituent(s);
provided that when X is a moiety of formula III and A is O, then n is 2, 3 or 4; when X is a moiety of formula III and A is S, then n is 2, 3 or 4; and when X is a moiety of formula III and A is (CR¹⁵R¹⁶)ₘS, then n is 0, 2, 3 or 4.

9. The method of claim 8, wherein:
X is a moiety of formula III;
n is 0, 1, 2 or 3;
Z is SH, SO₃H, SO₂H , SOH or S(NHR¹²)₂R¹³; and
A is O, S or CR¹⁵R¹⁶.

10. The method of claim 9, wherein Z is SH.

11. The method of claim 10, wherein R⁸ is —(CH₂)₂COOH.

12. The method of claim 10, wherein the compound of formula II is selected from:
2-(2-sulfanylethyl)pentanedioic acid;
3-(2-sulfanylethyl)-1,3,5-pentanetricarboxylic acid;
2-(2-sulfanylpropyl)pentanedioic acid;
2-(2-sulfanylbutyl)pentanedioic acid;
2-(2-sulfanyl-2-phenylethyl)pentanedioic acid;
2-(2-sulfanylhexyl)pentanedioic acid;
2-(2-sulfanyl-1-methylethyl)pentanedioic acid;
2-[1-(sulfanylmethyl)propyl]pentanedioic acid;
2-(3-sulfanylpentyl)pentanedioic acid;
2-(3-sulfanylpropyl)pentanedioic acid;
2-(3-sulfanyl-2-methylpropyl)pentanedioic acid;
2-(3-sulfanyl-2-phenylpropyl)pentanedioic acid;
2-(3-sulfanylbutyl)pentanedioic acid;
2-[3-sulfanyl-2-(phenylmethyl)propyl]pentanedioic acid;
2-[2-(sulfanylmethyl)butyl]pentanedioic acid;
2-[2-(sulfanylmethyl)pentyl]pentanedioic acid;
2-(3-sulfanyl-4-methylpentyl)pentanedioic acid; and
enantiomers and pharmaceutically acceptable equivalents.

13. The method of claim 1, wherein the NAALADase inhibitor is a compound of formula VI 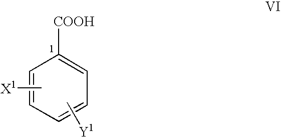
or an enantiomer or a pharmaceutically acceptable equivalent of said compound, wherein:
X¹ is —W-Z¹;
W is a bond or a linking group;
Z¹ is a terminal group; and
Y¹ is —COOH oriented meta or para relative to C-1.

14. The method of claim 13, wherein:
X¹ is -(CR¹⁷R¹⁸)ₙNH(CR¹⁹R²⁰)ₘCOOH, —PO(OH)OR²², —(CR¹⁷R¹⁸)ₙP(O)(OH)R²², —NH—(CR¹⁹R²⁰)ₘ-heteroaryl, —NH(P(O)(R²³)OH), —(CR¹⁷R¹⁸)ₙNH(P(O)(OH)R²³), —CON(²²)(OH) —(CR¹⁷CR¹⁸)ₙCON(R²²)(OH), —(CR¹⁷CR¹⁸)ₙSH or —O(CR¹⁹R²⁰)ₘSH, —SO₂NH-aryl, —N(C═O)—CH₂(C═O)-aryl, —SO₂NH-aryl, —N(C═O)—CH₂(C═O)-aryl, -O-aryl wherein aryl in -O-aryl is substituted by at least one of nitro, carboxy or 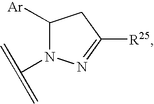
wherein X¹ is oriented meta or para relative to C-1;
m and n are independently 1-3, provided that when X¹ is —O(CR¹⁹R²⁰)ₘSH, then m is 2 or 3;
R¹⁷, R¹⁸, R¹⁹, R²⁰, R²², R²³ and R²⁵ are independently hydrogen, C₁-C₆ alkyl, C₂-C₆ alkenyl, C₂-C₆ alkynyl, aryl, heteroaryl, carbocycle, heterocycle, halo, hydroxy, sulfhydryl, nitro, amino or C₁-C₆ alkoxy, wherein said alkyl, alkenyl, alkynyl, aryl, heteroaryl, carbocycle, heterocycle and alkoxy are independently unsubstituted or substituted with one or more substituent(s); and
Y¹ is —COOH oriented meta or para relative to C-1.

15. The method of claim 13, wherein the compound of formula VI is selected from:
2-[(4-carboxyphenyl)sulfonyl]-I,4-benzene-dicarboxylic acid;
2-[(2,5-dicarboxyphenyl)sulfonyl]-1,4-benzene-dicarboxylic acid;
1,2,4-benzenetricarboxylic acid;
2-[(2-carboxyphenyl)thio]-1,4-benzenedicarboxylic acid;
2-nitro-1,4-benzenedicarboxylic acid;
2-bromo-1,4-benzenedicarboxylic acid;
2-amino-1,4-benzenedicarboxylic acid;
2-sulfoterephthalic acid, monosodium salt;
2-carboxymethyl-1,4-benzenedicarboxylic acid;
2-[(2-furanylmethyl)-amino]-1,4-benzenedicarboxylic acid;
2-[(carboxymethyl)amino]-1,4-benzenedicarboxylic acid;
4-(4-nitrobenzoyl)-1,3-benzenedicarboxylic acid;
4-[4-(2,4-dicarboxybenzoyl)phenoxy]-1,2-benzene-dicarboxylic acid;
4-[[(2,4,6-trimethylphenyl)amino]carbonyl]-1,3-benzenedicarboxylic acid;
4-nitro-1,3-benzenedicarboxylic acid;
4-[(1-naphthalenylamino)-carbonyl]-1,3-benzene-dicarboxylic acid;
1,2,4-benzenetricarboxylic acid;
4-[(2-carboxyphenyl)thio]-1,3-benzenedicarboxylic acid;
4-[3-[[3-(2,4-dicarboxyphenoxy)propyl]dithio]-propoxy]-1,3-benzenedicarboxylic acid;
4-hydroxy-1,3-benzenedicarboxylic acid;
4-[(2-furanylmethyl)amino]-1,3-benzenedicarboxylic acid;
4-(2-mercaptoethyl)-1,3-benzenedicarboxylic acid;
5-[4,5-dihydro-5-(4-hydroxyphenyl)-3-phenyl-1H-pyrazol-1-yl]-1,3-benzenedicarboxylic acid;
5-(4,5-dihydro-3-methyl-5-phenyl-1 H-pyrazol-1-yl)-1,3-benzenedicarboxylic acid;
5-[[(4-chloro-3-nitrophenyl)amino]sulfonyl]-1,3-benzenedicarboxylic acid;
5-[[[4-chloro-3-[[3-(2-methoxyphenyl)-1,3-dioxopropyl]amino]phenyl]amino]sulfonyl-1,3-benzenedicarboxylic acid;
5-[[3-[4-(acetylamino)phenyl]-1,3-dioxopropyl]amino]-1,3-benzenedicarboxylic acid;
5-acetylamino-1,3-benzenedicarboxylic acid;
5-[[(1-hydroxy-2-naphthalenyl)carbonyl]-methylamino]-1,3-benzenedicarboxylic acid;
5-(4-carboxy-2-nitrophenoxy)-1,3-benzenedicarboxylic acid;
5-sulfo-1,3-benzenedicarboxylic acid;
5-nitro-1,3-benzenedicarboxylic acid;
5-amino-1,3-benzenedicarboxylic acid;
1,3,5-benzenetricarboxylic acid;
5-[[(3-amino-4-chlorophenyl)amino]sulfonyl]-1,3-benzenedicarboxylic acid;
5-(3-mercaptopropoxy)-1,3-benzenedicarboxylic acid;
5-hydroxy-1,3-benzenedicarboxylic acid;
5-(2-mercaptoethoxy)-1,3-benzenedicarboxylic acid;
5-[(hydroxyamino)carbonyl]-1,3-benzenedicarboxylic acid;
5-phosphono-1,3-benzenedicarboxylic acid;
5-mercaptomethyl-1,3-benzenedicarboxylic acid;
5-phosphonomethyl-1,3-benzenedicarboxylic acid;
5-[[(carboxymethyl)amino]-methyl]-1,3-benzene-dicarboxylic acid;
5-[(carboxymethyl)amino]-1,3-benzenedicarboxylic acid;
5-[[(2-furanylmethyl)amino]-methyl]-1,3-benzene-dicarboxylic acid;
5-[2-(hydroxyamino)-2-oxoethyl]-1,3-benzene-dicarboxylic acid;
5-(2-mercaptoethyl)-1,3-benzenedicarboxylic acid; and
enantiomers and pharmaceutically acceptable equivalents.

16. The method of claim 1, wherein the NAALADase inhibitor is a compound of formula VII 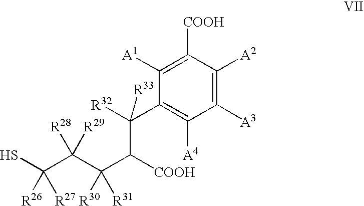
or an enantiomer or a pharmaceutically acceptable equivalent of said compound, wherein:
R²⁶, R²⁷, R²⁸, R²⁹, R³⁰, R³¹, R³² and R³³ are independently hydrogen or C₁-C₃ alkyl;
A¹, A², A³ and A⁴ are independently hydrogen, C₁-C₆ alkyl, C₁-C₆ alkoxy, halo, nitro, phenyl, phenoxy, benzyl, benzyloxy or —COOH, or any adjacent two of A², A³ and A⁴ form with the benzene ring a fused 5- or 6-membered carbocyclic or heterocyclic aromatic ring, said heterocyclic aromatic ring containing 1 or 2 oxygen, nitrogen and/or sulfur heteroatom(s).

17. The method of claim 16, wherein:
R²⁶, R²⁷, R²⁸, R²⁹, R³⁰, R³¹, R³² and R³³ are independently hydrogen or methyl; and
A¹, A², A³ and A⁴ are independently hydrogen, C-C₄ alkyl, C₁-C₂ alkoxy, halo, nitro, phenyl, phenoxy, benzyloxy, nitro or —COOH.

18. The method of claim 16, wherein any adjacent two of A², A³ and A⁴ form with the benzene ring a fused 5- or 6-membered carbocyclic or heterocyclic aromatic ring, said heterocyclic aromatic ring containing 1 or 2 oxygen, nitrogen and/or sulfur heteroatom(s).

19. The method of claim 1, wherein the NAALADase inhibitor is a compound of formula VIII 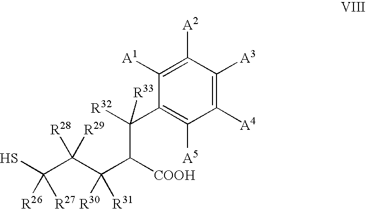
or an enantiomer or a pharmaceutically acceptable equivalent of said compound, wherein:
R²⁶, R²⁷, R²⁸, R²⁹, R³⁰, R³¹, R³² and R³³ are independently hydrogen or C₁-C₃ alkyl; and
A¹, A², A³, A⁴ and A⁵ are independently hydrogen, C₁-C₆ alkyl, C₁-C₆ alkoxy, C₁-C₃ perhaloalkyl, phenyl, phenoxy, benzyl, benzyloxy, hydroxy, halo, cyano, nitro, —SO₂R³⁴, —(C═O)NR³⁴R³⁵, —(C═O)NR³⁴(CH₂)ₙCOOH, —NR³⁴(C═O)R³⁵, —(CH₂)ₙCOOH or —COOH, or any adjacent two of A¹, A², A³, A⁴ and As form with the benzene ring a fused 5- or 6-membered carbocyclic or heterocyclic aromatic ring, said heterocyclic aromatic ring containing 1 or 2 oxygen, nitrogen and/or sulfur heteroatom(s);
R³⁴ and R³⁵ are independently hydrogen, C₁-C₆ alkyl, phenyl or benzyl; and
n is 1-3.

20. The method of claim 19, wherein:
R²⁶, R²⁷, R²⁸, R²⁹, R³⁰, R³¹, R³² and R³³ are each hydrogen;
A¹, A², A³, A⁴ and A⁵ are independently hydrogen, C₁-C₄ alkyl, C₁-C₂ alkoxy, C₁-C₂ perhaloalkyl, phenyl, phenoxy, hydroxy, halo, cyano, nitro, —SO₂R³⁴, —(C═O)NR³⁴R³⁵, —(C═O)NR³⁴(CH₂)COOH, —NR³⁴(C═O)R³⁵ or —(CH₂)COOH; and
R³⁴ and R³⁵ are independently hydrogen, methyl or benzyl.

21. The method of claim 19, wherein any adjacent two of A¹, A², A³, A⁴ and A⁵ form with the benzene ring a fused 5- or 6-membered carbocyclic or heterocyclic aromatic ring, said heterocyclic aromatic ring containing 1 or 2 oxygen, nitrogen and/or sulfur heteroatom(s).

22. The method of claim 1, wherein the NAALADase inhibitor is a compound of formula IX 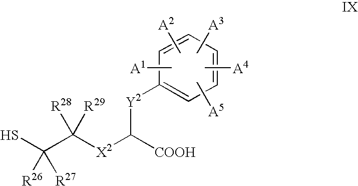
or an enantiomer or a pharmaceutically acceptable equivalent of said compound, wherein:
X² and Y² are independently —CR³⁰R³¹-, —O—, —S— or —NR³⁰-, provided that at least one of X² and Y² is/are —CR³⁰R³¹-;
A¹, A², A³, A⁴ and A⁵ are independently hydrogen, C₁-C₉ alkyl, C₂-C₉ alkenyl, C₂-C₉ alkynyl, aryl, heteroaryl, carbocycle, heterocycle, C₁-C₉ alkoxy, C₂-C₉ alkenyloxy, phenoxy, benzyloxy, hydroxy, halo, nitro, cyano, isocyano, —COOR³⁴, —COR³⁴, —NR³⁴R³⁵, —SR³⁴, —SOR³⁴, —SO₂R³⁴, —SO₂(OR³⁴), —(C═O)NR³⁴R³⁵, —(C═O)NR³⁴(CH₂),COOH, —NR³⁴(C═O)R³⁵ or —(CH₂)ₙCOOH, or any adjacent two of A¹, A², A³, A⁴ and A⁵ form with the benzene ring a fused ring that is saturated or unsaturated, aromatic or non-aromatic, and carbocyclic or heterocyclic, said heterocyclic ring containing 1 or 2 oxygen, nitrogen and/or sulfur heteroatom(s);
n is 1-3;
R²⁶, R²⁷, R²⁸, R²⁹, R³⁰, R³¹, R³⁴ and R³⁵ are independently hydrogen, C₁-C₉ alkyl, C₂-C₉ alkenyl, C₂-C₉ alkynyl, aryl, heteroaryl, carbocycle or heterocycle; and said alkyl, alkenyl, alkynyl, aryl, heteroaryl, carbocycle, heterocycle, alkoxy, alkenyloxy, phenoxy, benzyloxy, and fused ring are independently unsubstituted or substituted with one or more substituent(s).

23. The method of claim 22, wherein:
Y² is —O—, —S— or —NR³⁰- ;
A¹, A², A³, A⁴ and A⁵ are independently hydrogen, C₁-C₄ alkyl, C₁-C₂ alkoxy, hydroxy, halo, —COOH, —COR³⁴, —NR³⁴(C═O)R³⁵ or —(CH₂)COOH; and
R³⁴ and R³⁵ are independently hydrogen or methyl.

24. The method of claim 22, wherein:
Y² is —CR³⁰R³¹;
A¹, A², A³ and A⁴ are each hydrogen; and
A⁵ is phenoxy, benzyloxy, aryl, heteroaryl, carbocycle or heterocycle, wherein said phenoxy and benzyloxy are substituted with —COOH, and said aryl, heteroaryl, carbocycle and heterocycle are independently substituted with one or more substituent(s) selected from cyano and —COOH.

25. The method of claim 1, wherein the NAALADase inhibitor is a compound of formula X 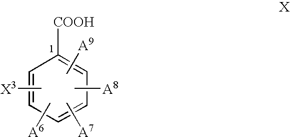
or an enantiomer or a pharmaceutically acceptable equivalent of said compound, wherein:
X³ is —(CR³⁶R³⁷)ₙSH, —O(CR³⁶R³⁷)₂SH, —S(CR³⁶R³⁷)₂SH or —NR(CR³⁶R³⁷)₂SH;
n is 1-3; and
R, R³⁶, R³⁷, A⁶, A⁷, A⁸ and A⁹ are independently hydrogen, C₁-C₉ alkyl, C₂-C₉ alkenyl, C₂-C₉ alkynyl, aryl, heteroaryl, carbocycle, heterocycle, halo, hydroxy, sulfhydryl, nitro, amino, cyano, isocyano, thiocyano, isothiocyano, formamido, thioformamido, sulfo, sulfino, C₁-C₉ alkylsulfonyl, C₁-C₉ alkoxy, C₂-C₉ alkenoxy, phenoxy or benzyloxy, wherein said alkyl, alkenyl, alkynyl, aryl, heteroaryl, carbocycle, heterocycle, alkoxy, alkenoxy, phenoxy and benzyloxy are independently unsubstituted or substituted with one or more substituent(s).

26. The method of claim 25, wherein the compound of formula X is selected from:
3-(2-mercaptoethyl)-benzoic acid;
3-(mercaptomethyl)-benzoic acid;
2-(mercaptomethyl)-benzoic acid;
5-hydroxy-2-(2-mercaptoethyl)-benzoic acid;
2-(2-mercaptoethyl)-benzoic acid;
5-[(4-carboxyphenyl)methoxy]-2-(2-mercaptoethyl)-benzoic acid;
2-(2-mercaptoethyl)-5-(phenylmethoxy)-benzoic acid;
2-(carboxymethoxy)-6-(2-mercaptoethyl)-benzoic acid;
5-[(3-carboxyphenyl)methoxy]-2-(2-mercaptoethyl)-benzoic acid;
2-(2-mercaptoethyl)-6-(phenylmethoxy)-benzoic acid;
2-[(2-carboxyphenyl)methoxy]-6-(2-mercaptoethyl)-benzoic acid;
2-[(4-carboxyphenyl)methoxy]-6-(2-mercaptoethyl)-benzoic acid;
3-(2-mercaptoethyl)-[1,1′-biphenyl]-2,3′-dicarboxylic acid;
2-(3,3-dimethylbutoxy)-6-(2-mercaptoethyl)-benzoic acid;
2-(2-mercaptoethyl)-6-(2-phenylethoxy)-benzoic acid;
2-[(2-chlorophenyl)methoxy]-6-(2-mercaptoethyl)-benzoic acid;
2-[[3-carboxy-5-(1,1-dimethylethyl)phenyl]methoxy]-6-(2-mercaptoethyl)-benzoic acid;
2-(2-mercaptoethyl)-6-phenoxy-benzoic acid;
2-(2-mercaptoethyl)-6-phenylamino-benzoic acid;
2-(2-mercaptoethyl)-6-(phenylthio)-benzoic acid;
5′-(1,1-dimethylethyl)-3-(2-mercaptoethyl)-[1,1′-biphenyl]-2,3′-dicarboxylic acid;
3-(2-mercaptoethyl)-[1,1′-biphenyl]-2,4′-dicarboxylic acid;
2-[(4-carboxy-2-methoxyphenyl)methoxy]-6-(2-mercaptoethyl)-benzoic acid;
2-[(4-carboxy-3-methoxyphenyl)methoxy]-6-(2-mercaptoethyl)-benzoic acid;
2-[(2-bromo-4-carboxyphenyl)methoxy]-6-(2-mercaptoethyl)-benzoic acid;
2-[(3-bromo-4-carboxyphenyl)methoxy]-6-(2-mercaptoethyl)-benzoic acid;
2-[(4-chlorophenyl)methoxy]-6-(2-mercaptoethyl)-benzoic acid;
2-(biphenyl-2-ylmethoxy)-6-(2-mercaptoethyl)-benzoic acid;
2-[(3-bromo-5-carboxyphenyl)methoxy]-6-(2-mercaptoethyl)-benzoic acid;
2-[(2-bromo-5-carboxyphenyl)methoxy]-6-(2-mercaptoethyl)-benzoic acid;
2-(2-mercaptoethyl)-6-[(4-methoxyphenyl)methoxy]-benzoic acid;
2-(2-mercaptoethyl)-6-[(4-methylphenyl)methoxy]-benzoic acid;
2-[(4-bromo-3-carboxyphenyl)methoxy]-6-(2-mercaptoethyl)-benzoic acid;
2-[(2-carboxy-5-methoxyphenyl)methoxy]-6-(2-mercaptoethyl)-benzoic acid;
5-(mercaptomethyl)-2-(2-phenylethoxy)-benzoic acid;
2-bromo-5-(mercaptomethyl)-benzoic acid;
4-(mercaptomethyl)-[1,1′-biphenyl]-2,3′-dicarboxylic acid;
5-(mercaptomethyl)-2-(phenylmethoxy)-benzoic acid; and
4-bromo-3-(mercaptomethyl)-benzoic acid; and
enantiomers and pharmaceutically acceptable equivalents.

27. The method of claim 1, wherein the NAALADase inhibitor is a compound of formula XI 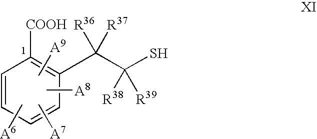
or an enantiomer or a pharmaceutically acceptable equivalent of said compound, wherein:
R³⁷, R³⁸, R³⁹ and R⁴⁰ are independently hydrogen or C₁-C₃ alkyl;
A⁶, A⁷, A⁸ and A⁹ are independently hydrogen, C₁-C₉ alkyl, C₂-C₉ alkenyl, C₂-C₉ alkynyl, aryl, heteroaryl, carbocycle, heterocycle, halo, hydroxy, sulfhydryl, nitro, amino, cyano, isocyano, thiocyano, isothiocyano, formamido, thioformamido, sulfo, sulfino, C₁-C₉ alkylsulfonyl, C₁-C₉ alkoxy, C₁-C₉ alkenoxy, phenoxy or benzyloxy, wherein said alkyl, alkenyl, alkynyl, aryl, heteroaryl, carbocycle, heterocycle, alkoxy, alkenoxy, phenoxy and benzyloxy are independently unsubstituted or substituted with one or more substituent(s).

28. The method of claim 27, wherein:
R³⁶, R³⁷, R³⁸ and R³⁹, A⁷, A⁸ and A⁹ are each hydrogen;
A⁶ is hydrogen, —(CH₂)ₙ-W¹, or -Y³-(CH₂)ₙ-W¹;
n is 0-3;
Y³ is O, S or NR⁴⁰;
R⁴⁰ is hydrogen or C₁-C₄ alkyl; and
W¹ is C₁-C₆ alkyl or phenyl, wherein W¹ is unsubstituted or substituted with C₁-C₄ alkyl, C₁-C₄ alkoxy, carboxy or halo.

29. The method of claim 1, wherein the NAALADase inhibitor is a compound of formula XII 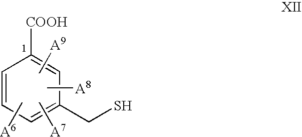
or an enantiomer or a pharmaceutically acceptable equivalent of said compound, wherein:
A⁶, A⁷, A⁸ and A⁹ are independently hydrogen, C₁-C₉ alkyl, C₂-C₉ alkenyl, C₂-C₉ alkynyl, aryl, heteroaryl, carbocycle, heterocycle, halo, hydroxy, sulfhydryl, nitro, amino, cyano, isocyano, thiocyano, isothiocyano, formamido, thioformamido, sulfo, sulfino, C₁-C₉ alkylsulfonyl, C₁-C₉ alkoxy, C₂-C₉ alkenoxy, phenoxy or benzyloxy, wherein said alkyl, alkenyl, alkynyl, aryl, heteroaryl, carbocycle, heterocycle, alkoxy, alkenoxy, phenoxy and benzyloxy are independently unsubstituted or substituted with one or more substituent(s).

30. The method of claim 29, wherein:
A⁷, A⁸ and A⁹ are each hydrogen;
A⁶is —(CH₂)ₙ-Ar² or -Y-(CH₂)ₙ-AR²;
n is 0-3;
Y³ is O, S or NR⁴¹;
R⁴¹ is hydrogen or C₁-C₄ alkyl; and
Ar is phenyl, wherein Ar² is unsubstituted or substituted with C₁-C₄ alkyl, carboxy or halo.

31. The method of claim 1, wherein the NAALADase inhibitor is a compound of formula XIII 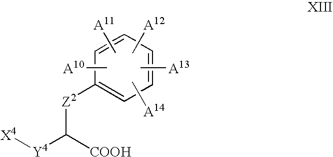
or an enantiomer or a pharmaceutically acceptable equivalent of said compound, wherein:
X⁴ is —(CO)NHOH or —N(OH)COH;
Y⁴ is a bond or a divalent linking group having from 1 to 9 carbon atom(s) and from 0 to 5 heteroatom(s) independently selected from oxygen, sulfur and nitrogen;
Z² is —CR⁴¹R⁴²-, —NR⁴¹-, —O— or —S—;
A¹⁰, A¹¹, A¹², A¹³ and A¹⁴ are independently hydrogen, C₁-C₉ alkyl, C₂-C₉ alkenyl, C₂-C₉ alkynyl, aryl, heteroaryl, carbocycle, heterocycle, C₁-C₉ alkoxy, C₂-C₉ alkenyloxy, phenoxy, benzyloxy, hydroxy, halo, nitro, cyano, isocyano, —COOR⁴³, COR⁴³, —NR⁴³R⁴⁴, —SR⁴³, —SOR⁴³, —SO₂R⁴³, —SO₂(0R⁴³), —(CO)NR⁴³R⁴³, —(CO)NR⁴³(CH₂)ₙCOOH, —NR⁴³(CO)R⁴⁴ or —(CH₂)ₙCOOH, or any adjacent two of A¹⁰, A¹¹, A¹² and A¹³ form with the benzene ring a fused ring that is saturated or unsaturated, aromatic or non-aromatic, and carbocyclic or heterocyclic, said heterocyclic ring containing 1 or 2 oxygen, nitrogen and/or sulfur heteroatom(s);
n is 1-3;
R⁴¹, R⁴², R⁴³ and R⁴⁴ are independently hydrogen, C₁-C₉ alkyl, C₂-C₉ alkenyl, C₂-C₉ alkynyl, aryl, heteroaryl, carbocycle or heterocycle; and
said alkyl, alkenyl, alkynyl, aryl, heteroaryl, carbocycle, heterocycle, alkoxy, alkenyloxy, phenoxy, benzyloxy, and fused ring are independently unsubstituted or substituted with one or more substituent(s).

32. The method of claim 31, wherein:
Y⁴ is —(CR⁴⁵R⁴⁶)ₚ-W²—(CR⁴⁷R⁴⁸)_{q}-;
W² is —CR⁴⁹R⁵⁰-, NR⁴⁹-, —O—, —S— or —SO₂—;
p and q are independently 0-4; provided that when q is 0 and W² is —NR⁴⁹-, —O—, —S— or —SO₂—, then Z² is —CR⁴¹R⁴²-;
R⁴⁵,R⁴⁶, R⁴⁷,R⁴⁸, R⁴⁹ and R⁵⁰ are independently hydrogen, C₁-C₉ alkyl, C₂-C₉ alkenyl, C₂-C₉ alkynyl, aryl, heteroaryl, carbocycle, heterocycle, halo, hydroxy, sulfhydryl, nitro, amino, cyano, isocyano, thiocyano, isothiocyano, formamido, thioformamido, sulfo, sulfino, C₁-C₉ alkoxy, C₂-C₉ alkenoxy, phenoxy or benzyloxy, wherein said alkyl, alkenyl, alkynyl, aryl, heteroaryl, carbocycle, heterocycle, alkoxy, alkenyloxy, phenoxy and benzyloxy are independently unsubstituted or substituted with one or more substituent(s); and
A¹⁰, A¹¹ and A¹² are each hydrogen.

33. The method of claim 32, wherein:
Y⁴ is —(CR⁴⁵R⁴⁶)ₚ-W²—(CR⁴⁷R⁴⁸)_{q}-;
W² is —CR⁴⁹R⁵⁰-;
p is 0-4;
q is 0;
R⁴⁵, R⁴⁶, R⁴⁷, R⁴⁸, R⁴⁹ and R⁵⁰ are each hydrogen;
A¹⁰, A¹¹ and A¹² are each hydrogen;
A¹³is hydrogen, —COOR⁴³, C₁-C₄ alkyl, C₂-C₄ alkenyl or C₂-C₄ alkynyl; and
A¹⁴ is —COOR⁴³.

34. The method of claim 32, wherein:
Y⁴ is —(CR⁴⁵R⁴⁶)ₚ-W²—(CR⁴⁷R⁴⁸)_{q}-;
W² is —S—;
p and q are independently 1-4;
R⁴⁵, R⁴⁶, R⁴⁷, R⁴⁸, R⁴⁹ and R⁵⁰ are independently hydrogen, C₁-C₄ alkyl, C₂-C₄ alkenyl, C₂-C₄ alkynyl or phenyl;
A¹⁰, A¹¹ and A¹² are each hydrogen;
A¹³ is hydrogen, C₁-C₄ alkyl, C₂-C₄ alkenyl, C₂-C₄ alkynyl, phenyl, benzyl, phenoxy, benzyloxy or halo, wherein said alkyl, alkenyl, alkynyl, phenyl, benzyl, phenoxy and benzyloxy are independently unsubstituted or substituted with carobxy; and
A¹⁴ is —COOH.

35. The method of claim 32, wherein:
Y⁴ is (CR⁴⁵R⁴⁶)ₚ-W²—(CR⁴⁷R⁴⁸)_{q}-;
W² is —CR⁴⁹R⁵⁰-, —NR⁴⁹-, —O—, —S— or —SO₂—;
p and q are independently 0-4, provided that when q is 0 and W² is —NR⁴⁹-, —O—, —S— or —SO₂—, then Z² is —CR⁴¹R⁴²-;
R⁴⁵, R⁴⁶, R⁴⁷, R⁴⁸, R⁴⁹ and R⁵⁰ are independently hydrogen, C₁-C₉ alkyl, C₂-C₉ alkenyl, C₂-C₉ alkynyl, aryl, heteroaryl, carbocycle, heterocycle, halo, hydroxy, sulfhydryl, nitro, amino, cyano, isocyano, thiocyano, isothiocyano, formamido, thioformamido, sulfo, sulfino, C₁-C₉ alkoxy, C₂-C₉ alkenoxy, phenoxy or benzyloxy, wherein said alkyl, alkenyl, alkynyl, aryl, heteroaryl, carbocycle, heterocycle, alkoxy, alkenyloxy, phenoxy and benzyloxy are independently unsubstituted or substituted with one or more substituent(s);
A¹⁰, A¹¹ and A¹² are each hydrogen;
A¹³ is hydrogen; and
A¹⁴ is benzyl or carboxybenzyl.

36. The method of claim 31, wherein the compound of formula XIII is selected from:
3-tert-butyl-5-(2-carboxy-3-hydroxycarbamoyl-propyl)-benzoic acid;
3-tert-butyl-5-(2-carboxy-4-hydroxycarbamoyl-butyl)-benzoic acid;
3-(2-carboxy-4-hydroxycarbamoyl-butyl)-benzoic acid;
3-(2-carboxy-5-hydroxycarbamoyl-pentyl)-benzoic acid;
3-(2-carboxy-3-hydroxycarbamoyl-propyl)-benzoic acid;
3-(2-carboxy-2-hydroxycarbamoyl-ethyl)-benzoic acid;
3-tert-butyl-5-(2-carboxy-2-hydroxycarbamoyl-ethyl)-benzoic acid;
3-tert-butyl-5-(2-carboxy-2-hydroxycarbamoyl-ethyl)-benzoic acid methyl ester;
3-(2-carboxy-3-hydroxyamino-propyl)-benzoic acid;
3-(2-carboxy-2-hydroxycarbamoyl-ethyl)-benzoic acid methyl ester;
3-(2-carboxy-5-hydroxycarbamoylmethylsulfanyl-pentyl)-benzoic acid;
3-[2-carboxy-5-(2-hydroxycarbamoyl-ethylsulfanyl)-pentyl]-benzoic acid;
3-[2-carboxy-5-(1-hydroxycarbamoyl-propylsulfanyl)-pentyl]-benzoic acid;
3-(2-carboxy-5-hydroxycarbamoylmethyl-sulfanylpentyl)-benzoic acid;
3-(2-carboxy-5-hydroxycarbamoylmethylsulfanyl-pentyl)-benzoic acid;
3-tert-butyl-5-(2-carboxy-4-hydroxycarbamoylmethyl-sulfanylbutyl)-benzoic acid;
3-[2-carboxy-5-(hydroxycarbamoylphenylmethyl-sulfanyl)pentyl]-benzoic acid;
3-[2-carboxy-5-(1-hydroxycarbamoylbutylsulfanyl)-pentyl]-benzoic acid;
5-(2-carboxy-5-hydroxycarbamoylmethylsulfanyl-pentyl)-biphenyl-3-carboxylic acid;
3-bromo-5-(2-carboxy-5-hydroxycarbamoylmethyl-sulfanylpentyl)-benzoic acid;
3-benzyloxy-5-(2-carboxy-5-hydroxycarbamoylmethyl-sulfanylpentyl)-benzoic acid;
3-[2-carboxy-5-(1-hydroxycarbamoyl-2-methyl-propylsulfanyl)-pentyl] -benzoic acid;
3-(2-carboxy-3-hydroxycarbamoylmethyl-sulfanylpropyl)-benzoic acid;
3-(2-carboxy-5-hydroxycarbamoylmethyl-sulfanylpentyl)-5-phenoxy-benzoic acid;
3-(2-carboxy-6-hydroxycarbamoylmethyl-sulfanylhexyl)-benzoic acid;
3-(2-carboxy-4-hydroxycarbamoylmethyl-sulfanylbutyl)-benzoic acid;
3-[2-carboxy-3-(3-hydroxycarbamoyl-propylsulfanyl)-propyl]-benzoic acid;
3-[2-carboxy-5-(4-hydroxycarbamoyl-butylsulfanyl)-pentyl]-benzoic acid;
3-{2-carboxy-5-[(hydroxy-methyl-carbamoyl)-methylsulfanyl]-pentyl}-benzoic acid;
3-tert-butyl-5- [2-carboxy-4-(1-hydroxycarbamoyl-propylsulfanyl)-butyl]-benzoic acid;
3-(2-carboxy-5-hydroxycarbamoylmethyl-sulfanylpentyl)-4-chloro-benzoic acid;
3-[2-carboxy-4-(1-hydroxycarbamoyl-propylsulfanyl)-butyl]-benzoic acid;
3-[2-carboxy-3-(1-hydroxycarbamoyl-propylsulfanyl)-propyl]-benzoic acid;
2-biphenyl-3-ylmethyl-5-hydroxycarbamoylmethyl-sulfanyl-pentanoic acid;
3′-(2-carboxy-5-hydroxycarbamoylmethylsulfanyl-pentyl)-biphenyl-3-carboxylic acid;
2-bromo-4-(2-carboxy-5-hydroxycarbamoylmethyl-sulfanylpentyl)-benzoic acid; and
enantiomers and pharmaceutically acceptable equivalents.

37. The method of claim 1, wherein the NAALADase inhibitor is a compound of formula XIV 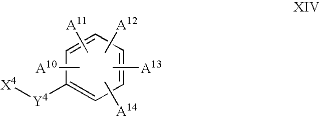
or an enantiomer or a pharmaceutically acceptable equivalent of said compound, wherein:
X⁴ is —(CO)NHOH or —N(OH)COH;
Y⁴ is a bond or a divalent linking group having from 1 to 9 carbon atom(s) and from 0 to 5 heteroatom(s) independently selected from oxygen, sulfur and nitrogen;
A¹⁰, A¹¹, A¹², A¹³ and A¹⁴ are independently hydrogen, C₁-C₉ alkyl, C₂-C₉ alkenyl, C₂-C₉ alkynyl, aryl, heteroaryl, carbocycle, heterocycle, C₁-C₉ alkoxy, C₂-C₉ alkenyloxy, phenoxy, benzyloxy, hydroxy, halo, nitro, cyano, isocyano, —COOR⁴³, —COR⁴³—NR⁴³R⁴⁴, —SR⁴³, —SOR⁴³, —SO₂R⁴³, —SO₂(OR⁴³), —(CO)NR⁴³R⁴⁴, —(CO)NR⁴³(CH₂)ₙCOOH, —NR⁴³(CO)R⁴⁴ or —(CH₂)ₙCOOH, or any adjacent two of A¹⁰, A¹¹, A¹² and A¹³ form with the benzene ring a fused ring that is saturated or unsaturated, aromatic or non-aromatic, and carbocyclic or heterocyclic, said heterocyclic ring containing 1 or 2 oxygen, nitrogen and/or sulfur heteroatom(s);
n is 1-3;
R⁴³ and R⁴⁴ are independently hydrogen, C₁-C₉ alkyl, C₂-C₉ alkenyl, C₂-C₉ alkynyl, aryl, heteroaryl, carbocycle or heterocycle; and
said alkyl, alkenyl, alkynyl, aryl, heteroaryl, carbocycle, heterocycle, alkoxy, alkenyloxy, phenoxy, benzyloxy, and fused ring are independently unsubstituted or substituted with one or more substituent(s).

38. The method of claim 37, wherein:
Y⁴ is a bond or —(CR⁴⁵R⁴⁶)ₚ-W²—(CR⁴⁷R⁴⁸ )_{q}-;
W² is —CR⁴⁹R⁵⁰-, —NR⁴⁹-, —O—, —S— or —SO₂—;
p and q are independently 0-4;
R⁴⁵, R⁴⁶, R⁴⁷, R⁴⁸, R⁴⁹ and R⁵⁰ are independently hydrogen, C₁-C₉ alkyl, C₂-C₉ alkenyl, C₂-C₉ alkynyl, aryl, heteroaryl, carbocycle, heterocycle, halo, hydroxy, sulfhydryl, nitro, amino, cyano, isocyano, thiocyano, isothiocyano, formamido, thioformamido, sulfo, sulfino, C₁-C₉ alkoxy, C₂-C₉ alkenoxy, phenoxy or benzyloxy, wherein said alkyl, alkenyl, alkynyl, aryl, heteroaryl, carbocycle, heterocycle, alkoxy, alkenyloxy, phenoxy and benzyloxy are independently unsubstituted or substituted with one or more substituent(s); and
A¹⁰, A¹¹ and A¹² are each hydrogen.

39. The method of claim 37, wherein:
Y⁴ is a bond;
A¹⁰, A¹¹ and A¹² are each hydrogen;
A¹³ is hydroxy, phenoxy, benzyloxy, —COOR⁴³ or —(CO)NHR⁴⁴;
A¹⁴ is —COOR⁴³;
R⁴³ is hydrogen, C₁-C₄ alkyl, C₂-C₄ alkenyl or C₂-C₄ alkynyl;
R⁴⁴ is benzyl; and
said benzyl, phenoxy and benzyloxy are independently unsubstituted or substituted with —COOR⁴³.

40. The method of claim 37, wherein:
Y⁴ is (CR⁴⁵R⁴⁶)ₚW²—(CR⁴⁷R⁴⁸)_{q}-;
W² is —O— or —S—; R⁴⁵, R⁴⁶, R⁴⁷ and R⁴⁸ are each hydrogen;
A¹⁰, A¹¹ and A¹² are each hydrogen;
A¹³ is hydrogen, —COOH, phenyl or benzyloxy, wherein said phenyl and benzyloxy are independently unsubstituted or substituted with —COOR⁴³; and
A¹⁴ is —COOR⁴³.

41. The method of claim 37, wherein:
Y⁴ is a bond or —(CR⁴⁵R⁴⁶)ₚ-W²—(CR⁴⁷R⁴⁸)_{q}-;
W² is —CR⁴⁹R⁵⁰-, —NR⁴⁹-, —O—, —S— or —SO₂—;
p and q are independently 0-4;
R⁴⁵, R⁴⁶, R⁴⁷, R⁴⁸, R⁴⁹ and R⁵⁰ are independently hydrogen, C₁-C₉ alkyl, C₂-C₉ alkenyl, C₂-C₉ alkynyl, aryl, heteroaryl, carbocycle, heterocycle, halo, hydroxy, sulfhydryl, nitro, amino, cyano, isocyano, thiocyano, isothiocyano, formamido, thioformamido, sulfo, sulfino, C₁-C₉ alkoxy, C₂-C₉ alkenoxy, phenoxy or benzyloxy, wherein said alkyl, alkenyl, alkynyl, aryl, heteroaryl, carbocycle, heterocycle, alkoxy, alkenyloxy, phenoxy and benzyloxy are independently unsubstituted or substituted with one or more substituent(s);
A¹⁰, A¹¹ and A¹² are each hydrogen;
A¹³ is hydrogen, nitro or C₁-C₄ alkoxy; and
A¹⁴ is hydroxy, phenoxy, benzyloxy, benzoyl or C₁-C₄ alkoxy, wherein said phenoxy, benzyloxy, benzoyl and alkoxy are independently unsubstituted or substituted with one or more substituent(s).

42. The method of claim 37, wherein the compound is selected from:
5-hydroxycarbamoyl-isophthalic acid monoethyl ester;
6-benzyloxy-N-hydroxy-isophthalamic acid methyl ester;
6,N-dihydroxy-isophthalamic acid;
6-benzyloxy-N-hydroxy-isophthalamic acid;
4-(3-hydroxycarbamoyl-propylsulfanylmethyl)-biphenyl-2,3′-dicarboxylic acid;
4-(4-hydroxycarbamoyl-butylsulfanylmethyl)-biphenyl-2,3′-dicarboxylic acid;
4-(2-hydroxycarbamoyl-ethylsulfanylmethyl)-biphenyl-2,3′-dicarboxylic acid;
3-(2-hydroxycarbamoyl-methylsulfanylethyl)-biphenyl-2,3′-dicarboxylic acid;
5-hydroxycarbamoylmethoxy-isophthalic acid;
3-hydroxycarbamoylmethoxy-benzoic acid;
3-(4-hydroxycarbamoyl-butoxy)-biphenyl-2,3′-dicarboxylic acid;
3-(4-hydroxycarbamoyl-butoxy)-biphenyl-2,3′-dicarboxylic acid;
3-(3-hydroxycarbamoyl-propoxy)-biphenyl-2,3′-dicarboxylic acid;
3-(2-hydroxycarbamoyl-ethoxy)-biphenyl-2,3′-dicarboxylic acid;
3-hydroxycarbamoylmethoxy-biphenyl-2,3′-dicarboxylic acid;
3-hydroxycarbamoylmethoxy-biphenyl-2,3′-dicarboxylic acid dimethyl ester;
2-hydroxycarbamoylmethoxy-benzoic acid;
2-hydroxycarbamoylmethoxy-benzoic acid methyl ester;
3-(2-hydroxycarbamoyl-ethoxy)-biphenyl-2,3′-dicarboxylic acid dimethyl ester;
4-(4-cyano-benzyloxy)-N-hydroxy-benzamide;
3-[3-(2-hydroxycarbamoyl-ethyl)-phenoxymethyl]-benzoic acid;
2,N-dihydroxy-benzamide;
4-(4-fluoro-phenoxy)-N-hydroxy-3-nitro-benzamide;
N-hydroxy-2,5-bis-(2,2,2-trifluoro-ethoxy)-benzamide;
N-hydroxy-2-(4-methyl-benzoyl)-benzamide; and
enantiomers and pharmaceutically acceptable equivalents.

43. The method of claim 1, wherein the NAALADase inhibitor is a compound of formula XV 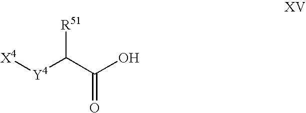
or an enantiomer or a pharmaceutically acceptable equivalent of said compound, wherein:
X⁴ is —(CO)NHOH or —N(OH)COH;
Y⁴ is a bond or a divalent linking group having from 1 to 9 carbon atom(s) and from 0 to 5 heteroatom(s) independently selected from oxygen, sulfur and nitrogen; and
R⁵¹ is hydrogen, C₁-C₉ alkyl, C₂-C₉ alkenyl, C₂-C₉ alkynyl, C₁-C₉ alkoxy or C₂-C₉ alkenoxy, wherein said alkyl, alkenyl, alkynyl, alkoxy and alkenoxy are independently unsubstituted or substituted with one or more substituent(s); provided that when Y is methylene, amine or oxygen, then R⁵¹ is not carboxyethyl.

44. The method of claim 43, wherein:
Y⁴ is —(CR⁴⁵R⁴⁶)ₚ-W²-(CR⁴⁷R⁴⁸)_{q}-;
W² is —CR⁴⁹R⁵⁰-, —NR⁴⁹-, —O—, —S— or —SO₂—;
p and q are independently 0-4; and
R⁴⁵, R⁴⁶, R⁴⁷, R⁴⁸, R⁴⁹ and R⁵⁰ are independently hydrogen, C₁-C₉ alkyl, C₂-C₉ alkenyl, C₂-C₉ alkynyl, aryl, heteroaryl, carbocycle, heterocycle, halo, hydroxy, sulfhydryl, nitro, amino, cyano, isocyano, thiocyano, isothiocyano, formamido, thioformamido, sulfo, sulfino, C₁-C₉ alkoxy, C₂-C₉ alkenoxy, phenoxy or benzyloxy, wherein said alkyl, alkenyl, alkynyl, aryl, heteroaryl, carbocycle, heterocycle, alkoxy, alkenyloxy, phenoxy and benzyloxy are independently unsubstituted or substituted with one or more substituent(s).

45. The method of claim 43, wherein:
Y⁴ is —(CR⁴⁵R⁴⁶)ₚ-W²—(CR⁴⁷R⁴⁸)_{q}-;
W² is —CR⁴⁹R⁵⁰- or —S—;
p is 0-1; q is 0-3; and
R⁴⁵, R⁴⁶, R⁴⁷, R⁴⁸, R⁴⁹ and R⁵⁰ are each hydrogen.

46. The method of claim 43, wherein the compound of formula XV is 2-(3-hydroxycarbamoyl-methylsulfanyl-propyl)-pentanedioic acid or an enantiomer or a pharmaceutically acceptable equivalent.

47. A pharmaceutical composition comprising:
(i) an effective amount of a NAALADase inhibitor for treating opioid tolerance; and
(ii) a pharmaceutically acceptable carrier.
